SEQUENCE LISTING (672 sequences)

SEQ ID NO: 1 - DNA, 12 bp, Artificial Sequence
  Synthetic sequence
cgacgtgtacgc

SEQ ID NO: 2 - DNA, 12 bp, Artificial Sequence
  Synthetic sequence
cgcctactcggt

SEQ ID NO: 3 - DNA, 12 bp, Artificial Sequence
  Synthetic sequence
gcctgtaagcgg

SEQ ID NO: 4 - DNA, 12 bp, Artificial Sequence
  Synthetic sequence
cccagaggttgg

SEQ ID NO: 5 - DNA, 12 bp, Artificial Sequence
  Synthetic sequence
tggatggcgtcg

SEQ ID NO: 6 - DNA, 12 bp, Artificial Sequence
  Synthetic sequence
gggactgatggg

SEQ ID NO: 7 - DNA, 12 bp, Artificial Sequence
  Synthetic sequence
gggaggagtcgc

SEQ ID NO: 8 - DNA, 12 bp, Artificial Sequence
  Synthetic sequence
gccgatcggacg

SEQ ID NO: 9 - DNA, 12 bp, Artificial Sequence
  Synthetic sequence
gtgtccgctctc

SEQ ID NO: 10 - DNA, 12 bp, Artificial Sequence
  Synthetic sequence
tctcgcggagct

SEQ ID NO: 11 - DNA, 12 bp, Artificial Sequence
  Synthetic sequence
ctgggccgagat

SEQ ID NO: 12 - DNA, 12 bp, Artificial Sequence
  Synthetic sequence
gtccgttcgggc

SEQ ID NO: 13 - DNA, 12 bp, Artificial Sequence
  Synthetic sequence
tcggcctgtggg

SEQ ID NO: 14 - DNA, 12 bp, Artificial Sequence
  Synthetic sequence
gacgatcctcgg

SEQ ID NO: 15 - DNA, 12 bp, Artificial Sequence
  Synthetic sequence
gagactgggccc

SEQ ID NO: 16 - DNA, 12 bp, Artificial Sequence
  Synthetic sequence
tcctctctgccg

SEQ ID NO: 17 - DNA, 12 bp, Artificial Sequence
  Synthetic sequence
cccagcttaggc

SEQ ID NO: 18 - DNA, 12 bp, Artificial Sequence
  Synthetic sequence
gggcttgcccat

SEQ ID NO: 19 - DNA, 12 bp, Artificial Sequence
  Synthetic sequence
gagggtctgtcg

SEQ ID NO: 20 - DNA, 12 bp, Artificial Sequence
  Synthetic sequence
tcctctctgccg

SEQ ID NO: 21 - DNA, 12 bp, Artificial Sequence
  Synthetic sequence
ccgtgtgttggg

SEQ ID NO: 22 - DNA, 12 bp, Artificial Sequence
  Synthetic sequence
cggttctctccc

SEQ ID NO: 23 - DNA, 12 bp, Artificial Sequence
  Synthetic sequence
ccggagttacgg

SEQ ID NO: 24 - DNA, 12 bp, Artificial Sequence
  Synthetic sequence
gcgctcatagcg

SEQ ID NO: 25 - DNA, 12 bp, Artificial Sequence
  Synthetic sequence
ggcagtgaacgg

SEQ ID NO: 26 - DNA, 12 bp, Artificial Sequence
  Synthetic sequence
ggcagggtaggc

SEQ ID NO: 27 - DNA, 12 bp, Artificial Sequence
  Synthetic sequence
cggtcgttcgct

SEQ ID NO: 28 - DNA, 12 bp, Artificial Sequence
  Synthetic sequence
cgtcatctcggg

SEQ ID NO: 29 - DNA, 12 bp, Artificial Sequence
  Synthetic sequence
gtccgcctgaac

SEQ ID NO: 30 - DNA, 12 bp, Artificial Sequence
  Synthetic sequence
ccgtgtggatcc

SEQ ID NO: 31 - DNA, 12 bp, Artificial Sequence
  Synthetic sequence
gggagcgggatc

SEQ ID NO: 32 - DNA, 12 bp, Artificial Sequence
  Synthetic sequence
tcgtggactgcg

SEQ ID NO: 33 - DNA, 12 bp, Artificial Sequence
  Synthetic sequence
cgacgtgtacgc

SEQ ID NO: 34 - DNA, 12 bp, Artificial Sequence
  Synthetic sequence
gcctgtaagcgg

SEQ ID NO: 35 - DNA, 12 bp, Artificial Sequence
  Synthetic sequence
cccagaggttgg

SEQ ID NO: 36 - DNA, 12 bp, Artificial Sequence
  Synthetic sequence
tggtacgagccc

SEQ ID NO: 37 - DNA, 12 bp, Artificial Sequence
  Synthetic sequence
gggatcagccgc

SEQ ID NO: 38 - DNA, 12 bp, Artificial Sequence
  Synthetic sequence
cctgcgcaccac

SEQ ID NO: 39 - DNA, 12 bp, Artificial Sequence
  Synthetic sequence
gcctacatgggc

SEQ ID NO: 40 - DNA, 12 bp, Artificial Sequence
  Synthetic sequence
cgtcacacaggg

SEQ ID NO: 41 - DNA, 12 bp, Artificial Sequence
  Synthetic sequence
gccgatctaccc

SEQ ID NO: 42 - DNA, 12 bp, Artificial Sequence
  Synthetic sequence
ggcagtcgagag

SEQ ID NO: 43 - DNA, 12 bp, Artificial Sequence
  Synthetic sequence
gtcatcgccctg

SEQ ID NO: 44 - DNA, 12 bp, Artificial Sequence
  Synthetic sequence
ccgcgggactat

SEQ ID NO: 45 - DNA, 12 bp, Artificial Sequence
  Synthetic sequence
ccgaagggcagt

SEQ ID NO: 46 - DNA, 12 bp, Artificial Sequence
  Synthetic sequence
cgtcccagatcg

SEQ ID NO: 47 - DNA, 12 bp, Artificial Sequence
  Synthetic sequence
ggattcctgcgg

SEQ ID NO: 48 - DNA, 12 bp, Artificial Sequence
  Synthetic sequence
gcagtgtcaggg

SEQ ID NO: 49 - DNA, 12 bp, Artificial Sequence
  Synthetic sequence
gcccaacgttcc

SEQ ID NO: 50 - DNA, 12 bp, Artificial Sequence
  Synthetic sequence
ggagggcatctg

SEQ ID NO: 51 - DNA, 12 bp, Artificial Sequence
  Synthetic sequence
tcgaaccgtcgc

SEQ ID NO: 52 - DNA, 12 bp, Artificial Sequence
  Synthetic sequence
cgaagaccctcg

SEQ ID NO: 53 - DNA, 12 bp, Artificial Sequence
  Synthetic sequence
gtccacgaacgg

SEQ ID NO: 54 - DNA, 12 bp, Artificial Sequence
  Synthetic sequence
ccgtgtggatcc

SEQ ID NO: 55 - DNA, 12 bp, Artificial Sequence
  Synthetic sequence
tgcgatgaggcg

SEQ ID NO: 56 - DNA, 12 bp, Artificial Sequence
  Synthetic sequence
ctgtccagtggg

SEQ ID NO: 57 - DNA, 12 bp, Artificial Sequence
  Synthetic sequence
gccttggtcgtg

SEQ ID NO: 58 - DNA, 12 bp, Artificial Sequence
  Synthetic sequence
tcgtgtcgccac

SEQ ID NO: 59 - DNA, 12 bp, Artificial Sequence
  Synthetic sequence
gacgcgcctgcg

SEQ ID NO: 60 - DNA, 12 bp, Artificial Sequence
  Synthetic sequence
tcagcggtcccg

SEQ ID NO: 61 - DNA, 12 bp, Artificial Sequence
  Synthetic sequence
cgcctctttgcg

SEQ ID NO: 62 - DNA, 12 bp, Artificial Sequence
  Synthetic sequence
cgcgcaaatggc

SEQ ID NO: 63 - DNA, 12 bp, Artificial Sequence
  Synthetic sequence
gttaggcggcgg

SEQ ID NO: 64 - DNA, 12 bp, Artificial Sequence
  Synthetic sequence
ccgctcagtgtc

SEQ ID NO: 65 - DNA, 12 bp, Artificial Sequence
  Synthetic sequence
gagggcaacggt

SEQ ID NO: 66 - DNA, 12 bp, Artificial Sequence
  Synthetic sequence
gcgtatcgtcgc

SEQ ID NO: 67 - DNA, 12 bp, Artificial Sequence
  Synthetic sequence
cggatcgaacgg

SEQ ID NO: 68 - DNA, 12 bp, Artificial Sequence
  Synthetic sequence
gcgtgcgacgac

SEQ ID NO: 69 - DNA, 12 bp, Artificial Sequence
  Synthetic sequence
ggcaagagggct

SEQ ID NO: 70 - DNA, 12 bp, Artificial Sequence
  Synthetic sequence
gagtggcgtcgt

SEQ ID NO: 71 - DNA, 12 bp, Artificial Sequence
  Synthetic sequence
ccgcagctagag

SEQ ID NO: 72 - DNA, 12 bp, Artificial Sequence
  Synthetic sequence
tcccatcagcgg

SEQ ID NO: 73 - DNA, 12 bp, Artificial Sequence
  Synthetic sequence
cgtgggttggac

SEQ ID NO: 74 - DNA, 12 bp, Artificial Sequence
  Synthetic sequence
tgggtaccgcgg

SEQ ID NO: 75 - DNA, 12 bp, Artificial Sequence
  Synthetic sequence
gggcttctgcct

SEQ ID NO: 76 - DNA, 12 bp, Artificial Sequence
  Synthetic sequence
cgcctactcggt

SEQ ID NO: 77 - DNA, 12 bp, Artificial Sequence
  Synthetic sequence
ggcctgcgagtc

SEQ ID NO: 78 - DNA, 12 bp, Artificial Sequence
  Synthetic sequence
tggatggcgtcg

SEQ ID NO: 79 - DNA, 12 bp, Artificial Sequence
  Synthetic sequence
gggactgatggg

SEQ ID NO: 80 - DNA, 12 bp, Artificial Sequence
  Synthetic sequence
cccaggatgggt

SEQ ID NO: 81 - DNA, 12 bp, Artificial Sequence
  Synthetic sequence
gccgatcggacg

SEQ ID NO: 82 - DNA, 12 bp, Artificial Sequence
  Synthetic sequence
gctggaggctag

SEQ ID NO: 83 - DNA, 12 bp, Artificial Sequence
  Synthetic sequence
gtgtccgctctc

SEQ ID NO: 84 - DNA, 12 bp, Artificial Sequence
  Synthetic sequence
gattccctccgc

SEQ ID NO: 85 - DNA, 12 bp, Artificial Sequence
  Synthetic sequence
gtggacagtccg

SEQ ID NO: 86 - DNA, 12 bp, Artificial Sequence
  Synthetic sequence
cgttgttggccg

SEQ ID NO: 87 - DNA, 12 bp, Artificial Sequence
  Synthetic sequence
gtgtccgtgacg

SEQ ID NO: 88 - DNA, 12 bp, Artificial Sequence
  Synthetic sequence
tcgggcgccgag

SEQ ID NO: 89 - DNA, 12 bp, Artificial Sequence
  Synthetic sequence
gtccgttcgggc

SEQ ID NO: 90 - DNA, 12 bp, Artificial Sequence
  Synthetic sequence
gccctctcgtcg

SEQ ID NO: 91 - DNA, 12 bp, Artificial Sequence
  Synthetic sequence
ctcgtcgtctcg

SEQ ID NO: 92 - DNA, 12 bp, Artificial Sequence
  Synthetic sequence
ccgtgtgttggg

SEQ ID NO: 93 - DNA, 12 bp, Artificial Sequence
  Synthetic sequence
cggttctctccc

SEQ ID NO: 94 - DNA, 12 bp, Artificial Sequence
  Synthetic sequence
gcggtggattgg

SEQ ID NO: 95 - DNA, 12 bp, Artificial Sequence
  Synthetic sequence
cggtggtccatc

SEQ ID NO: 96 - DNA, 12 bp, Artificial Sequence
  Synthetic sequence
ccctcagttccg

SEQ ID NO: 97 - DNA, 12 bp, Artificial Sequence
  Synthetic sequence
cccagcttaggc

SEQ ID NO: 98 - DNA, 12 bp, Artificial Sequence
  Synthetic sequence
ccaagtgcgcac

SEQ ID NO: 99 - DNA, 12 bp, Artificial Sequence
  Synthetic sequence
tcctctctgccg

SEQ ID NO: 100 - DNA, 12 bp, Artificial Sequence
  Synthetic sequence
ccgtgtgttggg

SEQ ID NO: 101 - DNA, 12 bp, Artificial Sequence
  Synthetic sequence
ggcagtgaacgg

SEQ ID NO: 102 - DNA, 12 bp, Artificial Sequence
  Synthetic sequence
gcgaccatctcg

SEQ ID NO: 103 - DNA, 12 bp, Artificial Sequence
  Synthetic sequence
cgaagtggcgtc

SEQ ID NO: 104 - DNA, 12 bp, Artificial Sequence
  Synthetic sequence
gctcgtccctgt

SEQ ID NO: 105 - DNA, 12 bp, Artificial Sequence
  Synthetic sequence
ggcagggtaggc

SEQ ID NO: 106 - DNA, 12 bp, Artificial Sequence
  Synthetic sequence
gggagccaagtc

SEQ ID NO: 107 - DNA, 12 bp, Artificial Sequence
  Synthetic sequence
gtcgggaaggct

SEQ ID NO: 108 - DNA, 12 bp, Artificial Sequence
  Synthetic sequence
cgtccttctccg

SEQ ID NO: 109 - DNA, 12 bp, Artificial Sequence
  Synthetic sequence
gcgtcgattggg

SEQ ID NO: 110 - DNA, 12 bp, Artificial Sequence
  Synthetic sequence
gtccacgaacgg

SEQ ID NO: 111 - DNA, 12 bp, Artificial Sequence
  Synthetic sequence
gggaggagtcgc

SEQ ID NO: 112 - DNA, 12 bp, Artificial Sequence
  Synthetic sequence
gccctctcgtcg

SEQ ID NO: 113 - DNA, 12 bp, Artificial Sequence
  Synthetic sequence
cgtgggttggac

SEQ ID NO: 114 - DNA, 12 bp, Artificial Sequence
  Synthetic sequence
gacgatcctcgg

SEQ ID NO: 115 - DNA, 12 bp, Artificial Sequence
  Synthetic sequence
gtcggcgttgac

SEQ ID NO: 116 - DNA, 12 bp, Artificial Sequence
  Synthetic sequence
cggtggtccatc

SEQ ID NO: 117 - DNA, 12 bp, Artificial Sequence
  Synthetic sequence
gcgtaacgcgtg

SEQ ID NO: 118 - DNA, 12 bp, Artificial Sequence
  Synthetic sequence
tcctcgacagcc

SEQ ID NO: 119 - DNA, 12 bp, Artificial Sequence
  Synthetic sequence
gcctcaatgccc

SEQ ID NO: 120 - DNA, 12 bp, Artificial Sequence
  Synthetic sequence
gggcttgcccat

SEQ ID NO: 121 - DNA, 12 bp, Artificial Sequence
  Synthetic sequence
gacgcagccctg

SEQ ID NO: 122 - DNA, 12 bp, Artificial Sequence
  Synthetic sequence
cggttctctccc

SEQ ID NO: 123 - DNA, 12 bp, Artificial Sequence
  Synthetic sequence
tcggcctgtggg

SEQ ID NO: 124 - DNA, 12 bp, Artificial Sequence
  Synthetic sequence
ccctaccctcct

SEQ ID NO: 125 - DNA, 12 bp, Artificial Sequence
  Synthetic sequence
ccgcagctagag

SEQ ID NO: 126 - DNA, 12 bp, Artificial Sequence
  Synthetic sequence
gggcacaagtgg

SEQ ID NO: 127 - DNA, 12 bp, Artificial Sequence
  Synthetic sequence
gccgtgagtctg

SEQ ID NO: 128 - DNA, 12 bp, Artificial Sequence
  Synthetic sequence
tcggtggtgtgc

SEQ ID NO: 129 - DNA, 12 bp, Artificial Sequence
  Synthetic sequence
gatggagcggtg

SEQ ID NO: 130 - DNA, 12 bp, Artificial Sequence
  Synthetic sequence
gtccgcctgaac

SEQ ID NO: 131 - DNA, 12 bp, Artificial Sequence
  Synthetic sequence
gtcatcgccctg

SEQ ID NO: 132 - DNA, 12 bp, Artificial Sequence
  Synthetic sequence
cgccctaatcgg

SEQ ID NO: 133 - DNA, 12 bp, Artificial Sequence
  Synthetic sequence
gattccctccgc

SEQ ID NO: 134 - DNA, 12 bp, Artificial Sequence
  Synthetic sequence
gcgacggctaac

SEQ ID NO: 135 - DNA, 12 bp, Artificial Sequence
  Synthetic sequence
cacggcctcgtt

SEQ ID NO: 136 - DNA, 12 bp, Artificial Sequence
  Synthetic sequence
cgggagaaaccc

SEQ ID NO: 137 - DNA, 12 bp, Artificial Sequence
  Synthetic sequence
ccctcagttccg

SEQ ID NO: 138 - DNA, 12 bp, Artificial Sequence
  Synthetic sequence
cgttgttggccg

SEQ ID NO: 139 - DNA, 12 bp, Artificial Sequence
  Synthetic sequence
gggtttccaggg

SEQ ID NO: 140 - DNA, 12 bp, Artificial Sequence
  Synthetic sequence
tcgaaccgtcgc

SEQ ID NO: 141 - DNA, 12 bp, Artificial Sequence
  Synthetic sequence
gagggtctgtcg

SEQ ID NO: 142 - DNA, 12 bp, Artificial Sequence
  Synthetic sequence
ggaggatggcgg

SEQ ID NO: 143 - DNA, 12 bp, Artificial Sequence
  Synthetic sequence
ccggagttacgg

SEQ ID NO: 144 - DNA, 12 bp, Artificial Sequence
  Synthetic sequence
gtgtccgctctc

SEQ ID NO: 145 - DNA, 12 bp, Artificial Sequence
  Synthetic sequence
tcagcggtcccg

SEQ ID NO: 146 - DNA, 12 bp, Artificial Sequence
  Synthetic sequence
gggagtttggcc

SEQ ID NO: 147 - DNA, 12 bp, Artificial Sequence
  Synthetic sequence
tgccgtcgggcc

SEQ ID NO: 148 - DNA, 12 bp, Artificial Sequence
  Synthetic sequence
cggtcgttcgct

SEQ ID NO: 149 - DNA, 12 bp, Artificial Sequence
  Synthetic sequence
gcctcgtgtgtg

SEQ ID NO: 150 - DNA, 12 bp, Artificial Sequence
  Synthetic sequence
tggtgggaagcg

SEQ ID NO: 151 - DNA, 12 bp, Artificial Sequence
  Synthetic sequence
gtggtccgtgtc

SEQ ID NO: 152 - DNA, 12 bp, Artificial Sequence
  Synthetic sequence
ctcggaatggcg

SEQ ID NO: 153 - DNA, 12 bp, Artificial Sequence
  Synthetic sequence
gcggacacggtt

SEQ ID NO: 154 - DNA, 12 bp, Artificial Sequence
  Synthetic sequence
cggtcatggacc

SEQ ID NO: 155 - DNA, 12 bp, Artificial Sequence
  Synthetic sequence
cgtgctctccgt

SEQ ID NO: 156 - DNA, 12 bp, Artificial Sequence
  Synthetic sequence
cgaagaccctcg

SEQ ID NO: 157 - DNA, 12 bp, Artificial Sequence
  Synthetic sequence
tcggtcgctccg

SEQ ID NO: 158 - DNA, 12 bp, Artificial Sequence
  Synthetic sequence
gcctctaggagg

SEQ ID NO: 159 - DNA, 12 bp, Artificial Sequence
  Synthetic sequence
gacgttcgaggg

SEQ ID NO: 160 - DNA, 12 bp, Artificial Sequence
  Synthetic sequence
ccgttcgcgttg

SEQ ID NO: 161 - DNA, 12 bp, Artificial Sequence
  Synthetic sequence
cgacgtgtacgc

SEQ ID NO: 162 - DNA, 12 bp, Artificial Sequence
  Synthetic sequence
ctgtccagtggg

SEQ ID NO: 163 - DNA, 12 bp, Artificial Sequence
  Synthetic sequence
gccttggtcgtg

SEQ ID NO: 164 - DNA, 12 bp, Artificial Sequence
  Synthetic sequence
cccgatttcgcg

SEQ ID NO: 165 - DNA, 12 bp, Artificial Sequence
  Synthetic sequence
gggactgatggg

SEQ ID NO: 166 - DNA, 12 bp, Artificial Sequence
  Synthetic sequence
cggagtctcgct

SEQ ID NO: 167 - DNA, 12 bp, Artificial Sequence
  Synthetic sequence
cacaccactccg

SEQ ID NO: 168 - DNA, 12 bp, Artificial Sequence
  Synthetic sequence
cctggacagggc

SEQ ID NO: 169 - DNA, 12 bp, Artificial Sequence
  Synthetic sequence
ccgtggttaccg

SEQ ID NO: 170 - DNA, 12 bp, Artificial Sequence
  Synthetic sequence
tcagcggtcccg

SEQ ID NO: 171 - DNA, 12 bp, Artificial Sequence
  Synthetic sequence
gcctcggtacgc

SEQ ID NO: 172 - DNA, 12 bp, Artificial Sequence
  Synthetic sequence
cccaaggtggcg

SEQ ID NO: 173 - DNA, 12 bp, Artificial Sequence
  Synthetic sequence
ggacagagctgg

SEQ ID NO: 174 - DNA, 12 bp, Artificial Sequence
  Synthetic sequence
gccgatctaccc

SEQ ID NO: 175 - DNA, 12 bp, Artificial Sequence
  Synthetic sequence
gtgtccaaggcc

SEQ ID NO: 176 - DNA, 12 bp, Artificial Sequence
  Synthetic sequence
ggaggatggcgg

SEQ ID NO: 177 - DNA, 12 bp, Artificial Sequence
  Synthetic sequence
ggcgggtgaagg

SEQ ID NO: 178 - DNA, 12 bp, Artificial Sequence
  Synthetic sequence
gccagtcctggg

SEQ ID NO: 179 - DNA, 12 bp, Artificial Sequence
  Synthetic sequence
cgttgttggccg

SEQ ID NO: 180 - DNA, 12 bp, Artificial Sequence
  Synthetic sequence
gtgtccgtgacg

SEQ ID NO: 181 - DNA, 12 bp, Artificial Sequence
  Synthetic sequence
cggacgttggct

SEQ ID NO: 182 - DNA, 12 bp, Artificial Sequence
  Synthetic sequence
cggctaacccgg

SEQ ID NO: 183 - DNA, 12 bp, Artificial Sequence
  Synthetic sequence
ctcccgatccag

SEQ ID NO: 184 - DNA, 12 bp, Artificial Sequence
  Synthetic sequence
cgtcagccctag

SEQ ID NO: 185 - DNA, 12 bp, Artificial Sequence
  Synthetic sequence
ccgcatgtgtgc

SEQ ID NO: 186 - DNA, 12 bp, Artificial Sequence
  Synthetic sequence
cttacgaccgcc

SEQ ID NO: 187 - DNA, 12 bp, Artificial Sequence
  Synthetic sequence
ccctctgaacgc

SEQ ID NO: 188 - DNA, 12 bp, Artificial Sequence
  Synthetic sequence
gccaaggcacgg

SEQ ID NO: 189 - DNA, 12 bp, Artificial Sequence
  Synthetic sequence
gcctctaggagg

SEQ ID NO: 190 - DNA, 12 bp, Artificial Sequence
  Synthetic sequence
tttcggaggcgg

SEQ ID NO: 191 - DNA, 12 bp, Artificial Sequence
  Synthetic sequence
ggcaagagggct

SEQ ID NO: 192 - DNA, 12 bp, Artificial Sequence
  Synthetic sequence
cgcggtgcaggt

SEQ ID NO: 193 - DNA, 12 bp, Artificial Sequence
  Synthetic sequence
tcctcggagtgc

SEQ ID NO: 194 - DNA, 12 bp, Artificial Sequence
  Synthetic sequence
tgcgatgaggcg

SEQ ID NO: 195 - DNA, 12 bp, Artificial Sequence
  Synthetic sequence
ggcctgcgagtc

SEQ ID NO: 196 - DNA, 12 bp, Artificial Sequence
  Synthetic sequence
tggatggcgtcg

SEQ ID NO: 197 - DNA, 12 bp, Artificial Sequence
  Synthetic sequence
gcccgcgcggtt

SEQ ID NO: 198 - DNA, 12 bp, Artificial Sequence
  Synthetic sequence
cggcttgatcgg

SEQ ID NO: 199 - DNA, 12 bp, Artificial Sequence
  Synthetic sequence
ctcggaatggcg

SEQ ID NO: 200 - DNA, 12 bp, Artificial Sequence
  Synthetic sequence
cccaggatgggt

SEQ ID NO: 201 - DNA, 12 bp, Artificial Sequence
  Synthetic sequence
tgccgtcgggcc

SEQ ID NO: 202 - DNA, 12 bp, Artificial Sequence
  Synthetic sequence
gggaggagtcgc

SEQ ID NO: 203 - DNA, 12 bp, Artificial Sequence
  Synthetic sequence
gccgatcggacg

SEQ ID NO: 204 - DNA, 12 bp, Artificial Sequence
  Synthetic sequence
cgcctctttgcg

SEQ ID NO: 205 - DNA, 12 bp, Artificial Sequence
  Synthetic sequence
cgtcacacaggg

SEQ ID NO: 206 - DNA, 12 bp, Artificial Sequence
  Synthetic sequence
gcgtttacgccc

SEQ ID NO: 207 - DNA, 12 bp, Artificial Sequence
  Synthetic sequence
gggtagcactgg

SEQ ID NO: 208 - DNA, 12 bp, Artificial Sequence
  Synthetic sequence
gttaggcggcgg

SEQ ID NO: 209 - DNA, 12 bp, Artificial Sequence
  Synthetic sequence
ctgtaggccggc

SEQ ID NO: 210 - DNA, 12 bp, Artificial Sequence
  Synthetic sequence
ccgctcagtgtc

SEQ ID NO: 211 - DNA, 12 bp, Artificial Sequence
  Synthetic sequence
ggctgatacgcc

SEQ ID NO: 212 - DNA, 12 bp, Artificial Sequence
  Synthetic sequence
ccgcgggactat

SEQ ID NO: 213 - DNA, 12 bp, Artificial Sequence
  Synthetic sequence
gggagccaagtc

SEQ ID NO: 214 - DNA, 12 bp, Artificial Sequence
  Synthetic sequence
cccaccggtcgt

SEQ ID NO: 215 - DNA, 12 bp, Artificial Sequence
  Synthetic sequence
gctcccgccctg

SEQ ID NO: 216 - DNA, 12 bp, Artificial Sequence
  Synthetic sequence
ccctaggatccg

SEQ ID NO: 217 - DNA, 12 bp, Artificial Sequence
  Synthetic sequence
tctcgcggagct

SEQ ID NO: 218 - DNA, 12 bp, Artificial Sequence
  Synthetic sequence
gcgtcgattggg

SEQ ID NO: 219 - DNA, 12 bp, Artificial Sequence
  Synthetic sequence
ctcggattccgg

SEQ ID NO: 220 - DNA, 12 bp, Artificial Sequence
  Synthetic sequence
ccggttgttgcc

SEQ ID NO: 221 - DNA, 12 bp, Artificial Sequence
  Synthetic sequence
cgtcccagatcg

SEQ ID NO: 222 - DNA, 12 bp, Artificial Sequence
  Synthetic sequence
gtccgttcgggc

SEQ ID NO: 223 - DNA, 12 bp, Artificial Sequence
  Synthetic sequence
tcgcgcaccttc

SEQ ID NO: 224 - DNA, 12 bp, Artificial Sequence
  Synthetic sequence
ggcatgcgtcac

SEQ ID NO: 225 - DNA, 12 bp, Artificial Sequence
  Synthetic sequence
gccctctcgtcg

SEQ ID NO: 226 - DNA, 12 bp, Artificial Sequence
  Synthetic sequence
ggcgatggtccg

SEQ ID NO: 227 - DNA, 12 bp, Artificial Sequence
  Synthetic sequence
cgcctactcggt

SEQ ID NO: 228 - DNA, 12 bp, Artificial Sequence
  Synthetic sequence
cccagaggttgg

SEQ ID NO: 229 - DNA, 12 bp, Artificial Sequence
  Synthetic sequence
cgctgcttccgc

SEQ ID NO: 230 - DNA, 12 bp, Artificial Sequence
  Synthetic sequence
ggctcgcccagt

SEQ ID NO: 231 - DNA, 12 bp, Artificial Sequence
  Synthetic sequence
gggatcagccgc

SEQ ID NO: 232 - DNA, 12 bp, Artificial Sequence
  Synthetic sequence
ggcagtgaacgg

SEQ ID NO: 233 - DNA, 12 bp, Artificial Sequence
  Synthetic sequence
cccgttcaccgt

SEQ ID NO: 234 - DNA, 12 bp, Artificial Sequence
  Synthetic sequence
gcgaagcatgcc

SEQ ID NO: 235 - DNA, 12 bp, Artificial Sequence
  Synthetic sequence
ttgggctgctcg

SEQ ID NO: 236 - DNA, 12 bp, Artificial Sequence
  Synthetic sequence
ctcccagtcgcc

SEQ ID NO: 237 - DNA, 12 bp, Artificial Sequence
  Synthetic sequence
ctgtgatcggcg

SEQ ID NO: 238 - DNA, 12 bp, Artificial Sequence
  Synthetic sequence
cgcgcaaatggc

SEQ ID NO: 239 - DNA, 12 bp, Artificial Sequence
  Synthetic sequence
gcgctcgacccg

SEQ ID NO: 240 - DNA, 12 bp, Artificial Sequence
  Synthetic sequence
cagcgacgctcc

SEQ ID NO: 241 - DNA, 12 bp, Artificial Sequence
  Synthetic sequence
caagcgtgcgac

SEQ ID NO: 242 - DNA, 12 bp, Artificial Sequence
  Synthetic sequence
gattccctccgc

SEQ ID NO: 243 - DNA, 12 bp, Artificial Sequence
  Synthetic sequence
gtggacagtccg

SEQ ID NO: 244 - DNA, 12 bp, Artificial Sequence
  Synthetic sequence
ccgcggaattgg

SEQ ID NO: 245 - DNA, 12 bp, Artificial Sequence
  Synthetic sequence
ggcccatcgtcg

SEQ ID NO: 246 - DNA, 12 bp, Artificial Sequence
  Synthetic sequence
cagccacatcgc

SEQ ID NO: 247 - DNA, 12 bp, Artificial Sequence
  Synthetic sequence
ttgcgggctgcc

SEQ ID NO: 248 - DNA, 12 bp, Artificial Sequence
  Synthetic sequence
ggccaacacagc

SEQ ID NO: 249 - DNA, 12 bp, Artificial Sequence
  Synthetic sequence
gacacaagccgc

SEQ ID NO: 250 - DNA, 12 bp, Artificial Sequence
  Synthetic sequence
ggcgcccatccg

SEQ ID NO: 251 - DNA, 12 bp, Artificial Sequence
  Synthetic sequence
tggatgtccgcc

SEQ ID NO: 252 - DNA, 12 bp, Artificial Sequence
  Synthetic sequence
gacctccagctc

SEQ ID NO: 253 - DNA, 12 bp, Artificial Sequence
  Synthetic sequence
tcgggcgccgag

SEQ ID NO: 254 - DNA, 12 bp, Artificial Sequence
  Synthetic sequence
gcgtgcgacgac

SEQ ID NO: 255 - DNA, 12 bp, Artificial Sequence
  Synthetic sequence
gtggtccgtgtc

SEQ ID NO: 256 - DNA, 12 bp, Artificial Sequence
  Synthetic sequence
ggcagggtaggc

SEQ ID NO: 257 - DNA, 12 bp, Artificial Sequence
  Synthetic sequence
gcgatagggctg

SEQ ID NO: 258 - DNA, 12 bp, Artificial Sequence
  Synthetic sequence
gggcacaagtgg

SEQ ID NO: 259 - DNA, 12 bp, Artificial Sequence
  Synthetic sequence
gatgcggcgtgg

SEQ ID NO: 260 - DNA, 12 bp, Artificial Sequence
  Synthetic sequence
gcctgtaagcgg

SEQ ID NO: 261 - DNA, 12 bp, Artificial Sequence
  Synthetic sequence
ccgtggagaagg

SEQ ID NO: 262 - DNA, 12 bp, Artificial Sequence
  Synthetic sequence
tggtacgagccc

SEQ ID NO: 263 - DNA, 12 bp, Artificial Sequence
  Synthetic sequence
tcgtgtcgccac

SEQ ID NO: 264 - DNA, 12 bp, Artificial Sequence
  Synthetic sequence
gacgcgcctgcg

SEQ ID NO: 265 - DNA, 12 bp, Artificial Sequence
  Synthetic sequence
cgcgcgtttggt

SEQ ID NO: 266 - DNA, 12 bp, Artificial Sequence
  Synthetic sequence
gtacctaccggg

SEQ ID NO: 267 - DNA, 12 bp, Artificial Sequence
  Synthetic sequence
ccgactgcagac

SEQ ID NO: 268 - DNA, 12 bp, Artificial Sequence
  Synthetic sequence
gtcatgccacgg

SEQ ID NO: 269 - DNA, 12 bp, Artificial Sequence
  Synthetic sequence
gcctacatgggc

SEQ ID NO: 270 - DNA, 12 bp, Artificial Sequence
  Synthetic sequence
gctggaggctag

SEQ ID NO: 271 - DNA, 12 bp, Artificial Sequence
  Synthetic sequence
gtgtccgctctc

SEQ ID NO: 272 - DNA, 12 bp, Artificial Sequence
  Synthetic sequence
gcgcgagttggt

SEQ ID NO: 273 - DNA, 12 bp, Artificial Sequence
  Synthetic sequence
gggaatgacgcc

SEQ ID NO: 274 - DNA, 12 bp, Artificial Sequence
  Synthetic sequence
gggtacgtagcg

SEQ ID NO: 275 - DNA, 12 bp, Artificial Sequence
  Synthetic sequence
gcgacggctaac

SEQ ID NO: 276 - DNA, 12 bp, Artificial Sequence
  Synthetic sequence
gggacgccgcgt

SEQ ID NO: 277 - DNA, 12 bp, Artificial Sequence
  Synthetic sequence
ccggatagcagg

SEQ ID NO: 278 - DNA, 12 bp, Artificial Sequence
  Synthetic sequence
gcgtatcgtcgc

SEQ ID NO: 279 - DNA, 12 bp, Artificial Sequence
  Synthetic sequence
ccgaagggcagt

SEQ ID NO: 280 - DNA, 12 bp, Artificial Sequence
  Synthetic sequence
tgtcctgcgcct

SEQ ID NO: 281 - DNA, 12 bp, Artificial Sequence
  Synthetic sequence
ctccaccatgcg

SEQ ID NO: 282 - DNA, 12 bp, Artificial Sequence
  Synthetic sequence
tcggtggtgtgc

SEQ ID NO: 283 - DNA, 12 bp, Artificial Sequence
  Synthetic sequence
cggatcgaacgg

SEQ ID NO: 284 - DNA, 12 bp, Artificial Sequence
  Synthetic sequence
gcggactctccc

SEQ ID NO: 285 - DNA, 12 bp, Artificial Sequence
  Synthetic sequence
cgccgtacatcc

SEQ ID NO: 286 - DNA, 12 bp, Artificial Sequence
  Synthetic sequence
cggcaggtctac

SEQ ID NO: 287 - DNA, 12 bp, Artificial Sequence
  Synthetic sequence
tgcccgcagtac

SEQ ID NO: 288 - DNA, 12 bp, Artificial Sequence
  Synthetic sequence
ggattcctgcgg

SEQ ID NO: 289 - DNA, 12 bp, Artificial Sequence
  Synthetic sequence
ccagttggcgtg

SEQ ID NO: 290 - DNA, 12 bp, Artificial Sequence
  Synthetic sequence
ccaggcagggtc

SEQ ID NO: 291 - DNA, 12 bp, Artificial Sequence
  Synthetic sequence
ggaaggacaggc

SEQ ID NO: 292 - DNA, 12 bp, Artificial Sequence
  Synthetic sequence
gcattcaccgcg

SEQ ID NO: 293 - DNA, 12 bp, Artificial Sequence
  Synthetic sequence
tccagcgcacag

SEQ ID NO: 294 - DNA, 12 bp, Artificial Sequence
  Synthetic sequence
cgcaccgagaac

SEQ ID NO: 295 - DNA, 12 bp, Artificial Sequence
  Synthetic sequence
ggagtaagggcg

SEQ ID NO: 296 - DNA, 12 bp, Artificial Sequence
  Synthetic sequence
gagtggcgtcgt

SEQ ID NO: 297 - DNA, 12 bp, Artificial Sequence
  Synthetic sequence
ctcgtcgtctcg

SEQ ID NO: 298 - DNA, 12 bp, Artificial Sequence
  Synthetic sequence
ccgcagctagag

SEQ ID NO: 299 - DNA, 12 bp, Artificial Sequence
  Synthetic sequence
gtactgggcagg

SEQ ID NO: 300 - DNA, 12 bp, Artificial Sequence
  Synthetic sequence
tgggaccaccac

SEQ ID NO: 301 - DNA, 12 bp, Artificial Sequence
  Synthetic sequence
cagttgcctggg

SEQ ID NO: 302 - DNA, 12 bp, Artificial Sequence
  Synthetic sequence
gatcgtgaggcc

SEQ ID NO: 303 - DNA, 12 bp, Artificial Sequence
  Synthetic sequence
gacgttcgaggg

SEQ ID NO: 304 - DNA, 12 bp, Artificial Sequence
  Synthetic sequence
ccgtgcacgtag

SEQ ID NO: 305 - DNA, 12 bp, Artificial Sequence
  Synthetic sequence
gccacgtaccct

SEQ ID NO: 306 - DNA, 12 bp, Artificial Sequence
  Synthetic sequence
cgggcatctcgc

SEQ ID NO: 307 - DNA, 12 bp, Artificial Sequence
  Synthetic sequence
tcagacggagcg

SEQ ID NO: 308 - DNA, 12 bp, Artificial Sequence
  Synthetic sequence
tggtgggaagcg

SEQ ID NO: 309 - DNA, 12 bp, Artificial Sequence
  Synthetic sequence
taggtcgggcgc

SEQ ID NO: 310 - DNA, 12 bp, Artificial Sequence
  Synthetic sequence
ggagggatcccg

SEQ ID NO: 311 - DNA, 12 bp, Artificial Sequence
  Synthetic sequence
gtcgtgcggtac

SEQ ID NO: 312 - DNA, 12 bp, Artificial Sequence
  Synthetic sequence
tactctgccggg

SEQ ID NO: 313 - DNA, 12 bp, Artificial Sequence
  Synthetic sequence
ttgcaccgacgg

SEQ ID NO: 314 - DNA, 12 bp, Artificial Sequence
  Synthetic sequence
gcctcgtgtgtg

SEQ ID NO: 315 - DNA, 12 bp, Artificial Sequence
  Synthetic sequence
gtccaggcgggt

SEQ ID NO: 316 - DNA, 12 bp, Artificial Sequence
  Synthetic sequence
ccggattcaggc

SEQ ID NO: 317 - DNA, 12 bp, Artificial Sequence
  Synthetic sequence
cccacctcccat

SEQ ID NO: 318 - DNA, 12 bp, Artificial Sequence
  Synthetic sequence
cccgtgttcgcc

SEQ ID NO: 319 - DNA, 12 bp, Artificial Sequence
  Synthetic sequence
gtcctagcagcg

SEQ ID NO: 320 - DNA, 12 bp, Artificial Sequence
  Synthetic sequence
ggctctatcggg

SEQ ID NO: 321 - DNA, 12 bp, Artificial Sequence
  Synthetic sequence
cgacgccgtccg

SEQ ID NO: 322 - DNA, 12 bp, Artificial Sequence
  Synthetic sequence
tgtcttgggcgg

SEQ ID NO: 323 - DNA, 12 bp, Artificial Sequence
  Synthetic sequence
gcgctacctggt

SEQ ID NO: 324 - DNA, 12 bp, Artificial Sequence
  Synthetic sequence
gtctcacgtgcc

SEQ ID NO: 325 - DNA, 12 bp, Artificial Sequence
  Synthetic sequence
gagggtctgtcg

SEQ ID NO: 326 - DNA, 12 bp, Artificial Sequence
  Synthetic sequence
ggcgaatacccg

SEQ ID NO: 327 - DNA, 12 bp, Artificial Sequence
  Synthetic sequence
gggccttggcgg

SEQ ID NO: 328 - DNA, 12 bp, Artificial Sequence
  Synthetic sequence
catgtgggaggg

SEQ ID NO: 329 - DNA, 12 bp, Artificial Sequence
  Synthetic sequence
tcgctctgacgc

SEQ ID NO: 330 - DNA, 12 bp, Artificial Sequence
  Synthetic sequence
ggctgcctacac

SEQ ID NO: 331 - DNA, 12 bp, Artificial Sequence
  Synthetic sequence
ctggccgtacac

SEQ ID NO: 332 - DNA, 12 bp, Artificial Sequence
  Synthetic sequence
ggagggcatctg

SEQ ID NO: 333 - DNA, 12 bp, Artificial Sequence
  Synthetic sequence
gaggacatcggc

SEQ ID NO: 334 - DNA, 12 bp, Artificial Sequence
  Synthetic sequence
cggagaagggtg

SEQ ID NO: 335 - DNA, 12 bp, Artificial Sequence
  Synthetic sequence
tggctccgcggt

SEQ ID NO: 336 - DNA, 12 bp, Artificial Sequence
  Synthetic sequence
cgtatcgtggcc

SEQ ID NO: 337 - DNA, 12 bp, Artificial Sequence
  Synthetic sequence
cgtcatctcggg

SEQ ID NO: 338 - DNA, 12 bp, Artificial Sequence
  Synthetic sequence
gtcggcgttgac

SEQ ID NO: 339 - DNA, 12 bp, Artificial Sequence
  Synthetic sequence
gccacttcgcgc

SEQ ID NO: 340 - DNA, 12 bp, Artificial Sequence
  Synthetic sequence
cgtgggttggac

SEQ ID NO: 341 - DNA, 12 bp, Artificial Sequence
  Synthetic sequence
tccatggaccgg

SEQ ID NO: 342 - DNA, 12 bp, Artificial Sequence
  Synthetic sequence
gggagtttggcc

SEQ ID NO: 343 - DNA, 12 bp, Artificial Sequence
  Synthetic sequence
tcgtagccgggt

SEQ ID NO: 344 - DNA, 12 bp, Artificial Sequence
  Synthetic sequence
ggtcactgtccc

SEQ ID NO: 345 - DNA, 12 bp, Artificial Sequence
  Synthetic sequence
gatggagcggtg

SEQ ID NO: 346 - DNA, 12 bp, Artificial Sequence
  Synthetic sequence
gaggatgccgcg

SEQ ID NO: 347 - DNA, 12 bp, Artificial Sequence
  Synthetic sequence
cggaacgatggc

SEQ ID NO: 348 - DNA, 12 bp, Artificial Sequence
  Synthetic sequence
gtaccggtggcg

SEQ ID NO: 349 - DNA, 12 bp, Artificial Sequence
  Synthetic sequence
ccatcagcaccg

SEQ ID NO: 350 - DNA, 12 bp, Artificial Sequence
  Synthetic sequence
cgccctaatcgg

SEQ ID NO: 351 - DNA, 12 bp, Artificial Sequence
  Synthetic sequence
tggcacgttcgc

SEQ ID NO: 352 - DNA, 12 bp, Artificial Sequence
  Synthetic sequence
ccgatcttcggc

SEQ ID NO: 353 - DNA, 12 bp, Artificial Sequence
  Synthetic sequence
gggccatttgcg

SEQ ID NO: 354 - DNA, 12 bp, Artificial Sequence
  Synthetic sequence
ctgggtcaagcc

SEQ ID NO: 355 - DNA, 12 bp, Artificial Sequence
  Synthetic sequence
gcagtgtcaggg

SEQ ID NO: 356 - DNA, 12 bp, Artificial Sequence
  Synthetic sequence
cgcccgaaaacg

SEQ ID NO: 357 - DNA, 12 bp, Artificial Sequence
  Synthetic sequence
gtgaccctgagg

SEQ ID NO: 358 - DNA, 12 bp, Artificial Sequence
  Synthetic sequence
gctggcgaagag

SEQ ID NO: 359 - DNA, 12 bp, Artificial Sequence
  Synthetic sequence
ccaagtgcgcac

SEQ ID NO: 360 - DNA, 12 bp, Artificial Sequence
  Synthetic sequence
cgcctagtggac

SEQ ID NO: 361 - DNA, 12 bp, Artificial Sequence
  Synthetic sequence
ccctaacaggcg

SEQ ID NO: 362 - DNA, 12 bp, Artificial Sequence
  Synthetic sequence
ccgtgtgttggg

SEQ ID NO: 363 - DNA, 12 bp, Artificial Sequence
  Synthetic sequence
tcccatcagcgg

SEQ ID NO: 364 - DNA, 12 bp, Artificial Sequence
  Synthetic sequence
cgcggtgactag

SEQ ID NO: 365 - DNA, 12 bp, Artificial Sequence
  Synthetic sequence
tcggcctgtggg

SEQ ID NO: 366 - DNA, 12 bp, Artificial Sequence
  Synthetic sequence
cccgaagaaggc

SEQ ID NO: 367 - DNA, 12 bp, Artificial Sequence
  Synthetic sequence
ccctcatccggc

SEQ ID NO: 368 - DNA, 12 bp, Artificial Sequence
  Synthetic sequence
gccactcgttgg

SEQ ID NO: 369 - DNA, 12 bp, Artificial Sequence
  Synthetic sequence
ggcaggaacgtg

SEQ ID NO: 370 - DNA, 12 bp, Artificial Sequence
  Synthetic sequence
ggagtctccagg

SEQ ID NO: 371 - DNA, 12 bp, Artificial Sequence
  Synthetic sequence
gacgatcctcgg

SEQ ID NO: 372 - DNA, 12 bp, Artificial Sequence
  Synthetic sequence
gcggtggattgg

SEQ ID NO: 373 - DNA, 12 bp, Artificial Sequence
  Synthetic sequence
cggtggtccatc

SEQ ID NO: 374 - DNA, 12 bp, Artificial Sequence
  Synthetic sequence
gagactgggccc

SEQ ID NO: 375 - DNA, 12 bp, Artificial Sequence
  Synthetic sequence
gagattccgcgc

SEQ ID NO: 376 - DNA, 12 bp, Artificial Sequence
  Synthetic sequence
ggatcgcgagct

SEQ ID NO: 377 - DNA, 12 bp, Artificial Sequence
  Synthetic sequence
gccgtgagtctg

SEQ ID NO: 378 - DNA, 12 bp, Artificial Sequence
  Synthetic sequence
ggaccggatagc

SEQ ID NO: 379 - DNA, 12 bp, Artificial Sequence
  Synthetic sequence
ccgtgtggatcc

SEQ ID NO: 380 - DNA, 12 bp, Artificial Sequence
  Synthetic sequence
cacggcctcgtt

SEQ ID NO: 381 - DNA, 12 bp, Artificial Sequence
  Synthetic sequence
gcgcgcattgtc

SEQ ID NO: 382 - DNA, 12 bp, Artificial Sequence
  Synthetic sequence
cggcaaggacag

SEQ ID NO: 383 - DNA, 12 bp, Artificial Sequence
  Synthetic sequence
gaagctccgtgc

SEQ ID NO: 384 - DNA, 12 bp, Artificial Sequence
  Synthetic sequence
cggtcatggacc

SEQ ID NO: 385 - DNA, 12 bp, Artificial Sequence
  Synthetic sequence
cggtcgtcgact

SEQ ID NO: 386 - DNA, 12 bp, Artificial Sequence
  Synthetic sequence
cccagcttaggc

SEQ ID NO: 387 - DNA, 12 bp, Artificial Sequence
  Synthetic sequence
gcgtaacgcgtg

SEQ ID NO: 388 - DNA, 12 bp, Artificial Sequence
  Synthetic sequence
tgcgggtctccg

SEQ ID NO: 389 - DNA, 12 bp, Artificial Sequence
  Synthetic sequence
ggcgctagtgtc

SEQ ID NO: 390 - DNA, 12 bp, Artificial Sequence
  Synthetic sequence
gcccaacgttcc

SEQ ID NO: 391 - DNA, 12 bp, Artificial Sequence
  Synthetic sequence
cctgtcatggcg

SEQ ID NO: 392 - DNA, 12 bp, Artificial Sequence
  Synthetic sequence
gacggtacgacg

SEQ ID NO: 393 - DNA, 12 bp, Artificial Sequence
  Synthetic sequence
caggtaggtggg

SEQ ID NO: 394 - DNA, 12 bp, Artificial Sequence
  Synthetic sequence
cgccaatggcgg

SEQ ID NO: 395 - DNA, 12 bp, Artificial Sequence
  Synthetic sequence
gtaccgtccgac

SEQ ID NO: 396 - DNA, 12 bp, Artificial Sequence
  Synthetic sequence
cgcagggaggtt

SEQ ID NO: 397 - DNA, 12 bp, Artificial Sequence
  Synthetic sequence
ccggtttctcgc

SEQ ID NO: 398 - DNA, 12 bp, Artificial Sequence
  Synthetic sequence
tcgaaccgtcgc

SEQ ID NO: 399 - DNA, 12 bp, Artificial Sequence
  Synthetic sequence
ccgttcgcgttg

SEQ ID NO: 400 - DNA, 12 bp, Artificial Sequence
  Synthetic sequence
gtccgcctgaac

SEQ ID NO: 401 - DNA, 12 bp, Artificial Sequence
  Synthetic sequence
gggtttccaggg

SEQ ID NO: 402 - DNA, 12 bp, Artificial Sequence
  Synthetic sequence
gcgatgctcctc

SEQ ID NO: 403 - DNA, 12 bp, Artificial Sequence
  Synthetic sequence
cgtgctctccgt

SEQ ID NO: 404 - DNA, 12 bp, Artificial Sequence
  Synthetic sequence
gcacagtaccgg

SEQ ID NO: 405 - DNA, 12 bp, Artificial Sequence
  Synthetic sequence
ggtgcgtaggag

SEQ ID NO: 406 - DNA, 12 bp, Artificial Sequence
  Synthetic sequence
tggcgaccaacg

SEQ ID NO: 407 - DNA, 12 bp, Artificial Sequence
  Synthetic sequence
ggacctggagag

SEQ ID NO: 408 - DNA, 12 bp, Artificial Sequence
  Synthetic sequence
cgtttgggtgcc

SEQ ID NO: 409 - DNA, 12 bp, Artificial Sequence
  Synthetic sequence
cctgttaccggc

SEQ ID NO: 410 - DNA, 12 bp, Artificial Sequence
  Synthetic sequence
gagccctatggg

SEQ ID NO: 411 - DNA, 12 bp, Artificial Sequence
  Synthetic sequence
cacccgcgtggt

SEQ ID NO: 412 - DNA, 12 bp, Artificial Sequence
  Synthetic sequence
gaaaacgcggcc

SEQ ID NO: 413 - DNA, 12 bp, Artificial Sequence
  Synthetic sequence
tgctgctgtccc

SEQ ID NO: 414 - DNA, 12 bp, Artificial Sequence
  Synthetic sequence
tggtggccaggt

SEQ ID NO: 415 - DNA, 12 bp, Artificial Sequence
  Synthetic sequence
tcgcttcgtgcg

SEQ ID NO: 416 - DNA, 12 bp, Artificial Sequence
  Synthetic sequence
cacgagccatcg

SEQ ID NO: 417 - DNA, 12 bp, Artificial Sequence
  Synthetic sequence
cccagcttaggc

SEQ ID NO: 418 - DNA, 12 bp, Artificial Sequence
  Synthetic sequence
tcgaaccgtcgc

SEQ ID NO: 419 - DNA, 12 bp, Artificial Sequence
  Synthetic sequence
cgcttggcaccg

SEQ ID NO: 420 - DNA, 12 bp, Artificial Sequence
  Synthetic sequence
ggaggatggcgg

SEQ ID NO: 421 - DNA, 12 bp, Artificial Sequence
  Synthetic sequence
ccggagttacgg

SEQ ID NO: 422 - DNA, 12 bp, Artificial Sequence
  Synthetic sequence
ggataccggctc

SEQ ID NO: 423 - DNA, 12 bp, Artificial Sequence
  Synthetic sequence
gtgtccgctctc

SEQ ID NO: 424 - DNA, 12 bp, Artificial Sequence
  Synthetic sequence
ccctaccctcct

SEQ ID NO: 425 - DNA, 12 bp, Artificial Sequence
  Synthetic sequence
ccgcatgtgtgc

SEQ ID NO: 426 - DNA, 12 bp, Artificial Sequence
  Synthetic sequence
cgaagtggcgtc

SEQ ID NO: 427 - DNA, 12 bp, Artificial Sequence
  Synthetic sequence
ccgcagctagag

SEQ ID NO: 428 - DNA, 12 bp, Artificial Sequence
  Synthetic sequence
gggcacaagtgg

SEQ ID NO: 429 - DNA, 12 bp, Artificial Sequence
  Synthetic sequence
ggcagggtaggc

SEQ ID NO: 430 - DNA, 12 bp, Artificial Sequence
  Synthetic sequence
cgtcatctcggg

SEQ ID NO: 431 - DNA, 12 bp, Artificial Sequence
  Synthetic sequence
tggtacgagccc

SEQ ID NO: 432 - DNA, 12 bp, Artificial Sequence
  Synthetic sequence
cgggtcaaacgg

SEQ ID NO: 433 - DNA, 12 bp, Artificial Sequence
  Synthetic sequence
tggatgtccgcc

SEQ ID NO: 434 - DNA, 12 bp, Artificial Sequence
  Synthetic sequence
cagacccgactc

SEQ ID NO: 435 - DNA, 12 bp, Artificial Sequence
  Synthetic sequence
tggtgggaagcg

SEQ ID NO: 436 - DNA, 12 bp, Artificial Sequence
  Synthetic sequence
gggatagtcggc

SEQ ID NO: 437 - DNA, 12 bp, Artificial Sequence
  Synthetic sequence
gggatcagccgc

SEQ ID NO: 438 - DNA, 12 bp, Artificial Sequence
  Synthetic sequence
ccgcagtgaacg

SEQ ID NO: 439 - DNA, 12 bp, Artificial Sequence
  Synthetic sequence
caggtaggtggg

SEQ ID NO: 440 - DNA, 12 bp, Artificial Sequence
  Synthetic sequence
ctgggtcaagcc

SEQ ID NO: 441 - DNA, 12 bp, Artificial Sequence
  Synthetic sequence
tcagcttcccgc

SEQ ID NO: 442 - DNA, 12 bp, Artificial Sequence
  Synthetic sequence
cggcttagacgg

SEQ ID NO: 443 - DNA, 12 bp, Artificial Sequence
  Synthetic sequence
cggttgtggcgc

SEQ ID NO: 444 - DNA, 12 bp, Artificial Sequence
  Synthetic sequence
cgcctactcggt

SEQ ID NO: 445 - DNA, 12 bp, Artificial Sequence
  Synthetic sequence
gcgtcgattggg

SEQ ID NO: 446 - DNA, 12 bp, Artificial Sequence
  Synthetic sequence
tgtcctgcgcct

SEQ ID NO: 447 - DNA, 12 bp, Artificial Sequence
  Synthetic sequence
tgtcttgggcgg

SEQ ID NO: 448 - DNA, 12 bp, Artificial Sequence
  Synthetic sequence
cccaaggtggcg

SEQ ID NO: 449 - DNA, 12 bp, Artificial Sequence
  Synthetic sequence
cgactggtaggc

SEQ ID NO: 450 - DNA, 12 bp, Artificial Sequence
  Synthetic sequence
gcctcaatgccc

SEQ ID NO: 451 - DNA, 12 bp, Artificial Sequence
  Synthetic sequence
ccaagtgcgcac

SEQ ID NO: 452 - DNA, 12 bp, Artificial Sequence
  Synthetic sequence
gagggtctgtcg

SEQ ID NO: 453 - DNA, 12 bp, Artificial Sequence
  Synthetic sequence
ccgtgtgttggg

SEQ ID NO: 454 - DNA, 12 bp, Artificial Sequence
  Synthetic sequence
gcccgtaacgcc

SEQ ID NO: 455 - DNA, 12 bp, Artificial Sequence
  Synthetic sequence
ggcagtgaacgg

SEQ ID NO: 456 - DNA, 12 bp, Artificial Sequence
  Synthetic sequence
ggcgctagtgtc

SEQ ID NO: 457 - DNA, 12 bp, Artificial Sequence
  Synthetic sequence
tcagcggtcccg

SEQ ID NO: 458 - DNA, 12 bp, Artificial Sequence
  Synthetic sequence
gggactgatggg

SEQ ID NO: 459 - DNA, 12 bp, Artificial Sequence
  Synthetic sequence
ggcctgccgagc

SEQ ID NO: 460 - DNA, 12 bp, Artificial Sequence
  Synthetic sequence
gggagtttggcc

SEQ ID NO: 461 - DNA, 12 bp, Artificial Sequence
  Synthetic sequence
tgccgtcgggcc

SEQ ID NO: 462 - DNA, 12 bp, Artificial Sequence
  Synthetic sequence
cgcagggaggtt

SEQ ID NO: 463 - DNA, 12 bp, Artificial Sequence
  Synthetic sequence
gggagccaagtc

SEQ ID NO: 464 - DNA, 12 bp, Artificial Sequence
  Synthetic sequence
tcggtggtgtgc

SEQ ID NO: 465 - DNA, 12 bp, Artificial Sequence
  Synthetic sequence
ccgtcaagaccg

SEQ ID NO: 466 - DNA, 12 bp, Artificial Sequence
  Synthetic sequence
cctgtcatggcg

SEQ ID NO: 467 - DNA, 12 bp, Artificial Sequence
  Synthetic sequence
tgcacaggacgg

SEQ ID NO: 468 - DNA, 12 bp, Artificial Sequence
  Synthetic sequence
cgtccttctccg

SEQ ID NO: 469 - DNA, 12 bp, Artificial Sequence
  Synthetic sequence
cccacgtcatcc

SEQ ID NO: 470 - DNA, 12 bp, Artificial Sequence
  Synthetic sequence
ccgcgggactat

SEQ ID NO: 471 - DNA, 12 bp, Artificial Sequence
  Synthetic sequence
gcccactcccag

SEQ ID NO: 472 - DNA, 12 bp, Artificial Sequence
  Synthetic sequence
tcccatcagcgg

SEQ ID NO: 473 - DNA, 12 bp, Artificial Sequence
  Synthetic sequence
tggctccgcggt

SEQ ID NO: 474 - DNA, 12 bp, Artificial Sequence
  Synthetic sequence
gtaccgtccgac

SEQ ID NO: 475 - DNA, 12 bp, Artificial Sequence
  Synthetic sequence
ggcatgcgtcac

SEQ ID NO: 476 - DNA, 12 bp, Artificial Sequence
  Synthetic sequence
ggaggagatggc

SEQ ID NO: 477 - DNA, 12 bp, Artificial Sequence
  Synthetic sequence
gagcggtaggtc

SEQ ID NO: 478 - DNA, 12 bp, Artificial Sequence
  Synthetic sequence
gccagtcctggg

SEQ ID NO: 479 - DNA, 12 bp, Artificial Sequence
  Synthetic sequence
tttcggaggcgg

SEQ ID NO: 480 - DNA, 12 bp, Artificial Sequence
  Synthetic sequence
ctcggaatggcg

SEQ ID NO: 481 - DNA, 12 bp, Artificial Sequence
  Synthetic sequence
ggcagtcgagag

SEQ ID NO: 482 - DNA, 12 bp, Artificial Sequence
  Synthetic sequence
cgccctaatcgg

SEQ ID NO: 483 - DNA, 12 bp, Artificial Sequence
  Synthetic sequence
cgccacccgcat

SEQ ID NO: 484 - DNA, 12 bp, Artificial Sequence
  Synthetic sequence
ctgggccgagat

SEQ ID NO: 485 - DNA, 12 bp, Artificial Sequence
  Synthetic sequence
tcctctctgccg

SEQ ID NO: 486 - DNA, 12 bp, Artificial Sequence
  Synthetic sequence
cggttctctccc

SEQ ID NO: 487 - DNA, 12 bp, Artificial Sequence
  Synthetic sequence
gagattccgcgc

SEQ ID NO: 488 - DNA, 12 bp, Artificial Sequence
  Synthetic sequence
tcggcctgtggg

SEQ ID NO: 489 - DNA, 12 bp, Artificial Sequence
  Synthetic sequence
gcgaccatctcg

SEQ ID NO: 490 - DNA, 12 bp, Artificial Sequence
  Synthetic sequence
cgcactgtcagc

SEQ ID NO: 491 - DNA, 12 bp, Artificial Sequence
  Synthetic sequence
gcgatgctcctc

SEQ ID NO: 492 - DNA, 12 bp, Artificial Sequence
  Synthetic sequence
ccgactgcagac

SEQ ID NO: 493 - DNA, 12 bp, Artificial Sequence
  Synthetic sequence
gctcgtccctgt

SEQ ID NO: 494 - DNA, 12 bp, Artificial Sequence
  Synthetic sequence
gctggcgaagag

SEQ ID NO: 495 - DNA, 12 bp, Artificial Sequence
  Synthetic sequence
gccgtgagtctg

SEQ ID NO: 496 - DNA, 12 bp, Artificial Sequence
  Synthetic sequence
gggccttggcgg

SEQ ID NO: 497 - DNA, 12 bp, Artificial Sequence
  Synthetic sequence
gcctcgtgtgtg

SEQ ID NO: 498 - DNA, 12 bp, Artificial Sequence
  Synthetic sequence
tctcgagggcac

SEQ ID NO: 499 - DNA, 12 bp, Artificial Sequence
  Synthetic sequence
gtcgggaaggct

SEQ ID NO: 500 - DNA, 12 bp, Artificial Sequence
  Synthetic sequence
gatggagcggtg

SEQ ID NO: 501 - DNA, 12 bp, Artificial Sequence
  Synthetic sequence
tccatggaccgg

SEQ ID NO: 502 - DNA, 12 bp, Artificial Sequence
  Synthetic sequence
gtccgcctgaac

SEQ ID NO: 503 - DNA, 12 bp, Artificial Sequence
  Synthetic sequence
gcgccactaagg

SEQ ID NO: 504 - DNA, 12 bp, Artificial Sequence
  Synthetic sequence
gtcgtgcggtac

SEQ ID NO: 505 - DNA, 12 bp, Artificial Sequence
  Synthetic sequence
gcgctacctggt

SEQ ID NO: 506 - DNA, 12 bp, Artificial Sequence
  Synthetic sequence
gtggtccgtgtc

SEQ ID NO: 507 - DNA, 12 bp, Artificial Sequence
  Synthetic sequence
gctggcacatgc

SEQ ID NO: 508 - DNA, 12 bp, Artificial Sequence
  Synthetic sequence
cgcggtgcaggt

SEQ ID NO: 509 - DNA, 12 bp, Artificial Sequence
  Synthetic sequence
gcgcgagttggt

SEQ ID NO: 510 - DNA, 12 bp, Artificial Sequence
  Synthetic sequence
cacgagccatcg

SEQ ID NO: 511 - DNA, 12 bp, Artificial Sequence
  Synthetic sequence
cccggtagaggt

SEQ ID NO: 512 - DNA, 12 bp, Artificial Sequence
  Synthetic sequence
ccctaggatccg

SEQ ID NO: 513 - DNA, 12 bp, Artificial Sequence
  Synthetic sequence
gtccacgaacgg

SEQ ID NO: 514 - DNA, 12 bp, Artificial Sequence
  Synthetic sequence
ggcaggaacgtg

SEQ ID NO: 515 - DNA, 12 bp, Artificial Sequence
  Synthetic sequence
tcgtagccgggt

SEQ ID NO: 516 - DNA, 12 bp, Artificial Sequence
  Synthetic sequence
gctcgagaaccc

SEQ ID NO: 517 - DNA, 12 bp, Artificial Sequence
  Synthetic sequence
gggcttgcccat

SEQ ID NO: 518 - DNA, 12 bp, Artificial Sequence
  Synthetic sequence
gacgcagccctg

SEQ ID NO: 519 - DNA, 12 bp, Artificial Sequence
  Synthetic sequence
tcgcgcaccttc

SEQ ID NO: 520 - DNA, 12 bp, Artificial Sequence
  Synthetic sequence
gcgctcatagcg

SEQ ID NO: 521 - DNA, 12 bp, Artificial Sequence
  Synthetic sequence
ccctctgaacgc

SEQ ID NO: 522 - DNA, 12 bp, Artificial Sequence
  Synthetic sequence
gagactgggccc

SEQ ID NO: 523 - DNA, 12 bp, Artificial Sequence
  Synthetic sequence
ggagggatcccg

SEQ ID NO: 524 - DNA, 12 bp, Artificial Sequence
  Synthetic sequence
cggaacgatggc

SEQ ID NO: 525 - DNA, 12 bp, Artificial Sequence
  Synthetic sequence
gggccatttgcg

SEQ ID NO: 526 - DNA, 12 bp, Artificial Sequence
  Synthetic sequence
gcacagtaccgg

SEQ ID NO: 527 - DNA, 12 bp, Artificial Sequence
  Synthetic sequence
ggcgttgcgact

SEQ ID NO: 528 - DNA, 12 bp, Artificial Sequence
  Synthetic sequence
cggtcgttcgct

SEQ ID NO: 529 - DNA, 12 bp, Artificial Sequence
  Synthetic sequence
tcgggcgccgag

SEQ ID NO: 530 - DNA, 12 bp, Artificial Sequence
  Synthetic sequence
tactctgccggg

SEQ ID NO: 531 - DNA, 12 bp, Artificial Sequence
  Synthetic sequence
gcccagtctgct

SEQ ID NO: 532 - DNA, 12 bp, Artificial Sequence
  Synthetic sequence
tggcacgttcgc

SEQ ID NO: 533 - DNA, 12 bp, Artificial Sequence
  Synthetic sequence
gtggtggccgcc

SEQ ID NO: 534 - DNA, 12 bp, Artificial Sequence
  Synthetic sequence
ctgccagctagc

SEQ ID NO: 535 - DNA, 12 bp, Artificial Sequence
  Synthetic sequence
ggtacgcaaggg

SEQ ID NO: 536 - DNA, 12 bp, Artificial Sequence
  Synthetic sequence
ccgtgtggatcc

SEQ ID NO: 537 - DNA, 12 bp, Artificial Sequence
  Synthetic sequence
gcgcgtcatgac

SEQ ID NO: 538 - DNA, 12 bp, Artificial Sequence
  Synthetic sequence
cgccgtacatcc

SEQ ID NO: 539 - DNA, 12 bp, Artificial Sequence
  Synthetic sequence
ggaccggatagc

SEQ ID NO: 540 - DNA, 12 bp, Artificial Sequence
  Synthetic sequence
ccggagtgtcag

SEQ ID NO: 541 - DNA, 12 bp, Artificial Sequence
  Synthetic sequence
ccgtggttaccg

SEQ ID NO: 542 - DNA, 12 bp, Artificial Sequence
  Synthetic sequence
tccggccctacg

SEQ ID NO: 543 - DNA, 12 bp, Artificial Sequence
  Synthetic sequence
tggtggccaggt

SEQ ID NO: 544 - DNA, 12 bp, Artificial Sequence
  Synthetic sequence
gtcatcgccctg

SEQ ID NO: 545 - DNA, 12 bp, Artificial Sequence
  Synthetic sequence
ggattcctgcgg

SEQ ID NO: 546 - DNA, 12 bp, Artificial Sequence
  Synthetic sequence
cccgtgttcgcc

SEQ ID NO: 547 - DNA, 12 bp, Artificial Sequence
  Synthetic sequence
gacggtacgacg

SEQ ID NO: 548 - DNA, 12 bp, Artificial Sequence
  Synthetic sequence
ggcccatcgtcg

SEQ ID NO: 549 - DNA, 12 bp, Artificial Sequence
  Synthetic sequence
gcggacacggtt

SEQ ID NO: 550 - DNA, 12 bp, Artificial Sequence
  Synthetic sequence
cggagtctcgct

SEQ ID NO: 551 - DNA, 12 bp, Artificial Sequence
  Synthetic sequence
gatgcggcgtgg

SEQ ID NO: 552 - DNA, 12 bp, Artificial Sequence
  Synthetic sequence
ctcgggcgcgct

SEQ ID NO: 553 - DNA, 12 bp, Artificial Sequence
  Synthetic sequence
cacaccactccg

SEQ ID NO: 554 - DNA, 12 bp, Artificial Sequence
  Synthetic sequence
gtcccacccgcc

SEQ ID NO: 555 - DNA, 12 bp, Artificial Sequence
  Synthetic sequence
gtccaggcgggt

SEQ ID NO: 556 - DNA, 12 bp, Artificial Sequence
  Synthetic sequence
cgggtgaaccgc

SEQ ID NO: 557 - DNA, 12 bp, Artificial Sequence
  Synthetic sequence
ccgtgcacgtag

SEQ ID NO: 558 - DNA, 12 bp, Artificial Sequence
  Synthetic sequence
cccgttcaccgt

SEQ ID NO: 559 - DNA, 12 bp, Artificial Sequence
  Synthetic sequence
ccaagctctcgg

SEQ ID NO: 560 - DNA, 12 bp, Artificial Sequence
  Synthetic sequence
cgtaacctcgcc

SEQ ID NO: 561 - DNA, 12 bp, Artificial Sequence
  Synthetic sequence
tcggcggaagtc

SEQ ID NO: 562 - DNA, 12 bp, Artificial Sequence
  Synthetic sequence
gacgatcctcgg

SEQ ID NO: 563 - DNA, 12 bp, Artificial Sequence
  Synthetic sequence
ctcggattccgg

SEQ ID NO: 564 - DNA, 12 bp, Artificial Sequence
  Synthetic sequence
gcctgtaagcgg

SEQ ID NO: 565 - DNA, 12 bp, Artificial Sequence
  Synthetic sequence
ggctgatacgcc

SEQ ID NO: 566 - DNA, 12 bp, Artificial Sequence
  Synthetic sequence
cccagagacgac

SEQ ID NO: 567 - DNA, 12 bp, Artificial Sequence
  Synthetic sequence
tgctgctgtccc

SEQ ID NO: 568 - DNA, 12 bp, Artificial Sequence
  Synthetic sequence
cgcctagtggac

SEQ ID NO: 569 - DNA, 12 bp, Artificial Sequence
  Synthetic sequence
ccagaagtcggc

SEQ ID NO: 570 - DNA, 12 bp, Artificial Sequence
  Synthetic sequence
tctgtgacgccc

SEQ ID NO: 571 - DNA, 12 bp, Artificial Sequence
  Synthetic sequence
gccttggtcgtg

SEQ ID NO: 572 - DNA, 12 bp, Artificial Sequence
  Synthetic sequence
cccgtttggacc

SEQ ID NO: 573 - DNA, 12 bp, Artificial Sequence
  Synthetic sequence
ggctctatcggg

SEQ ID NO: 574 - DNA, 12 bp, Artificial Sequence
  Synthetic sequence
cgtcccagatcg

SEQ ID NO: 575 - DNA, 12 bp, Artificial Sequence
  Synthetic sequence
ccatcagcaccg

SEQ ID NO: 576 - DNA, 12 bp, Artificial Sequence
  Synthetic sequence
gcgtaacgcgtg

SEQ ID NO: 577 - DNA, 12 bp, Artificial Sequence
  Synthetic sequence
gagggcaacggt

SEQ ID NO: 578 - DNA, 12 bp, Artificial Sequence
  Synthetic sequence
ccgttcgcgttg

SEQ ID NO: 579 - DNA, 12 bp, Artificial Sequence
  Synthetic sequence
tcctcgacagcc

SEQ ID NO: 580 - DNA, 12 bp, Artificial Sequence
  Synthetic sequence
ggtgctgtgctg

SEQ ID NO: 581 - DNA, 12 bp, Artificial Sequence
  Synthetic sequence
caagcgtgcgac

SEQ ID NO: 582 - DNA, 12 bp, Artificial Sequence
  Synthetic sequence
gcgatagggctg

SEQ ID NO: 583 - DNA, 12 bp, Artificial Sequence
  Synthetic sequence
gtgcacggctac

SEQ ID NO: 584 - DNA, 12 bp, Artificial Sequence
  Synthetic sequence
ccagttggcgtg

SEQ ID NO: 585 - DNA, 12 bp, Artificial Sequence
  Synthetic sequence
ccgtagtcagcg

SEQ ID NO: 586 - DNA, 12 bp, Artificial Sequence
  Synthetic sequence
cacggagtaccc

SEQ ID NO: 587 - DNA, 12 bp, Artificial Sequence
  Synthetic sequence
cggtcatggacc

SEQ ID NO: 588 - DNA, 12 bp, Artificial Sequence
  Synthetic sequence
ttggcctcacgc

SEQ ID NO: 589 - DNA, 12 bp, Artificial Sequence
  Synthetic sequence
catttgcgcggg

SEQ ID NO: 590 - DNA, 12 bp, Artificial Sequence
  Synthetic sequence
gcaaagcgaggc

SEQ ID NO: 591 - DNA, 12 bp, Artificial Sequence
  Synthetic sequence
gttaggcggcgg

SEQ ID NO: 592 - DNA, 12 bp, Artificial Sequence
  Synthetic sequence
cgggcatctcgc

SEQ ID NO: 593 - DNA, 12 bp, Artificial Sequence
  Synthetic sequence
cgggagaaaccc

SEQ ID NO: 594 - DNA, 12 bp, Artificial Sequence
  Synthetic sequence
cgacgtggtacc

SEQ ID NO: 595 - DNA, 12 bp, Artificial Sequence
  Synthetic sequence
tcggtcgctccg

SEQ ID NO: 596 - DNA, 12 bp, Artificial Sequence
  Synthetic sequence
tcagacggagcg

SEQ ID NO: 597 - DNA, 12 bp, Artificial Sequence
  Synthetic sequence
gagccctatggg

SEQ ID NO: 598 - DNA, 12 bp, Artificial Sequence
  Synthetic sequence
cggtcgtcgact

SEQ ID NO: 599 - DNA, 12 bp, Artificial Sequence
  Synthetic sequence
gccctttgcacg

SEQ ID NO: 600 - DNA, 12 bp, Artificial Sequence
  Synthetic sequence
cgccggcaattc

SEQ ID NO: 601 - DNA, 12 bp, Artificial Sequence
  Synthetic sequence
gtccgttcgggc

SEQ ID NO: 602 - DNA, 12 bp, Artificial Sequence
  Synthetic sequence
cgagccgacgac

SEQ ID NO: 603 - DNA, 12 bp, Artificial Sequence
  Synthetic sequence
ggcctgcgagtc

SEQ ID NO: 604 - DNA, 12 bp, Artificial Sequence
  Synthetic sequence
cgttgttggccg

SEQ ID NO: 605 - DNA, 12 bp, Artificial Sequence
  Synthetic sequence
tggcgaccaacg

SEQ ID NO: 606 - DNA, 12 bp, Artificial Sequence
  Synthetic sequence
gtcatgccacgg

SEQ ID NO: 607 - DNA, 12 bp, Artificial Sequence
  Synthetic sequence
gggtttccaggg

SEQ ID NO: 608 - DNA, 12 bp, Artificial Sequence
  Synthetic sequence
ccgtgagcttgc

SEQ ID NO: 609 - DNA, 12 bp, Artificial Sequence
  Synthetic sequence
ctggccgtacac

SEQ ID NO: 610 - DNA, 12 bp, Artificial Sequence
  Synthetic sequence
gtcctagcagcg

SEQ ID NO: 611 - DNA, 12 bp, Artificial Sequence
  Synthetic sequence
gccaaggcacgg

SEQ ID NO: 612 - DNA, 12 bp, Artificial Sequence
  Synthetic sequence
ggcgcccatccg

SEQ ID NO: 613 - DNA, 12 bp, Artificial Sequence
  Synthetic sequence
cgtatcgtggcc

SEQ ID NO: 614 - DNA, 12 bp, Artificial Sequence
  Synthetic sequence
cgcctctttgcg

SEQ ID NO: 615 - DNA, 12 bp, Artificial Sequence
  Synthetic sequence
cctgttaccggc

SEQ ID NO: 616 - DNA, 12 bp, Artificial Sequence
  Synthetic sequence
gattccctccgc

SEQ ID NO: 617 - DNA, 12 bp, Artificial Sequence
  Synthetic sequence
tcgtggactgcg

SEQ ID NO: 618 - DNA, 12 bp, Artificial Sequence
  Synthetic sequence
gccctctcgtcg

SEQ ID NO: 619 - DNA, 12 bp, Artificial Sequence
  Synthetic sequence
cacgtcccagag

SEQ ID NO: 620 - DNA, 12 bp, Artificial Sequence
  Synthetic sequence
gcctacatgggc

SEQ ID NO: 621 - DNA, 12 bp, Artificial Sequence
  Synthetic sequence
cacggcctcgtt

SEQ ID NO: 622 - DNA, 12 bp, Artificial Sequence
  Synthetic sequence
gtgtccaaggcc

SEQ ID NO: 623 - DNA, 12 bp, Artificial Sequence
  Synthetic sequence
ctcgtcgtctcg

SEQ ID NO: 624 - DNA, 12 bp, Artificial Sequence
  Synthetic sequence
cgccaatggcgg

SEQ ID NO: 625 - DNA, 12 bp, Artificial Sequence
  Synthetic sequence
ggcgtaatcgcc

SEQ ID NO: 626 - DNA, 12 bp, Artificial Sequence
  Synthetic sequence
gtcggcgttgac

SEQ ID NO: 627 - DNA, 12 bp, Artificial Sequence
  Synthetic sequence
ccgatcttcggc

SEQ ID NO: 628 - DNA, 12 bp, Artificial Sequence
  Synthetic sequence
cgacggctccat

SEQ ID NO: 629 - DNA, 12 bp, Artificial Sequence
  Synthetic sequence
gtctcacgtgcc

SEQ ID NO: 630 - DNA, 12 bp, Artificial Sequence
  Synthetic sequence
ctcacctgggcc

SEQ ID NO: 631 - DNA, 12 bp, Artificial Sequence
  Synthetic sequence
gcggactctccc

SEQ ID NO: 632 - DNA, 12 bp, Artificial Sequence
  Synthetic sequence
ggcatccggacg

SEQ ID NO: 633 - DNA, 12 bp, Artificial Sequence
  Synthetic sequence
ggagggcatctg

SEQ ID NO: 634 - DNA, 12 bp, Artificial Sequence
  Synthetic sequence
ccggatagcagg

SEQ ID NO: 635 - DNA, 12 bp, Artificial Sequence
  Synthetic sequence
gctcccgccctg

SEQ ID NO: 636 - DNA, 12 bp, Artificial Sequence
  Synthetic sequence
cggcttgatcgg

SEQ ID NO: 637 - DNA, 12 bp, Artificial Sequence
  Synthetic sequence
cgacgccgtccg

SEQ ID NO: 638 - DNA, 12 bp, Artificial Sequence
  Synthetic sequence
ccgctcagtgtc

SEQ ID NO: 639 - DNA, 12 bp, Artificial Sequence
  Synthetic sequence
ctgtccagtggg

SEQ ID NO: 640 - DNA, 12 bp, Artificial Sequence
  Synthetic sequence
ccggattcaggc

SEQ ID NO: 641 - DNA, 12 bp, Artificial Sequence
  Synthetic sequence
tgagcacgagcg

SEQ ID NO: 642 - DNA, 12 bp, Artificial Sequence
  Synthetic sequence
tcgtatgcgcgc

SEQ ID NO: 643 - DNA, 12 bp, Artificial Sequence
  Synthetic sequence
gcgtatcgtcgc

SEQ ID NO: 644 - DNA, 12 bp, Artificial Sequence
  Synthetic sequence
ggcgaatacccg

SEQ ID NO: 645 - DNA, 12 bp, Artificial Sequence
  Synthetic sequence
cgggacctgtac

SEQ ID NO: 646 - DNA, 12 bp, Artificial Sequence
  Synthetic sequence
cccgatttcgcg

SEQ ID NO: 647 - DNA, 12 bp, Artificial Sequence
  Synthetic sequence
cggacgttggct

SEQ ID NO: 648 - DNA, 12 bp, Artificial Sequence
  Synthetic sequence
tcctcggagtgc

SEQ ID NO: 649 - DNA, 12 bp, Artificial Sequence
  Synthetic sequence
gcgacggctaac

SEQ ID NO: 650 - DNA, 12 bp, Artificial Sequence
  Synthetic sequence
gcggtggattgg

SEQ ID NO: 651 - DNA, 12 bp, Artificial Sequence
  Synthetic sequence
cgtgggttggac

SEQ ID NO: 652 - DNA, 12 bp, Artificial Sequence
  Synthetic sequence
cggcaggtctac

SEQ ID NO: 653 - DNA, 12 bp, Artificial Sequence
  Synthetic sequence
cgtttgggtgcc

SEQ ID NO: 654 - DNA, 12 bp, Artificial Sequence
  Synthetic sequence
gcgcgcattgtc

SEQ ID NO: 655 - DNA, 12 bp, Artificial Sequence
  Synthetic sequence
gccactcgttgg

SEQ ID NO: 656 - DNA, 12 bp, Artificial Sequence
  Synthetic sequence
gcagtgtcaggg

SEQ ID NO: 657 - DNA, 12 bp, Artificial Sequence
  Synthetic sequence
cgggaacttgcg

SEQ ID NO: 658 - DNA, 12 bp, Artificial Sequence
  Synthetic sequence
gccgatcggacg

SEQ ID NO: 659 - DNA, 12 bp, Artificial Sequence
  Synthetic sequence
cggagaagggtg

SEQ ID NO: 660 - DNA, 12 bp, Artificial Sequence
  Synthetic sequence
ctccaccatgcg

SEQ ID NO: 661 - DNA, 12 bp, Artificial Sequence
  Synthetic sequence
ccctcatccggc

SEQ ID NO: 662 - DNA, 12 bp, Artificial Sequence
  Synthetic sequence
ggtgactgggag

SEQ ID NO: 663 - DNA, 12 bp, Artificial Sequence
  Synthetic sequence
gcgtgcgacgac

SEQ ID NO: 664 - DNA, 12 bp, Artificial Sequence
  Synthetic sequence
cggtggtccatc

SEQ ID NO: 665 - DNA, 12 bp, Artificial Sequence
  Synthetic sequence
gatcgtgaggcc

SEQ ID NO: 666 - DNA, 12 bp, Artificial Sequence
  Synthetic sequence
gacgttcgaggg

SEQ ID NO: 667 - DNA, 12 bp, Artificial Sequence
  Synthetic sequence
ggagtctccagg

SEQ ID NO: 668 - DNA, 12 bp, Artificial Sequence
  Synthetic sequence
tggatggcgtcg

SEQ ID NO: 669 - DNA, 12 bp, Artificial Sequence
  Synthetic sequence
gcccgcgcggtt

SEQ ID NO: 670 - DNA, 12 bp, Artificial Sequence
  Synthetic sequence
gtgtccgtgacg

SEQ ID NO: 671 - DNA, 12 bp, Artificial Sequence
  Synthetic sequence
cgctcaggcgtc

SEQ ID NO: 672 - DNA, 12 bp, Artificial Sequence
  Synthetic sequence
tagtccggaccc

(19) United States
(12) Patent Application Publication
(10) Pub. No.: US 2023/0419331 A1
(43) Pub. Date: Dec. 28, 2023

(54) OLIGONUCLEOTIDES REPRESENTING DIGITAL DATA

(71) Applicant: Nucleotrace Pty. Ltd., Toongabbie, Victoria (AU)

(72) Inventors: Nicholas Owen, Toongabbie, Victoria (AU); Marris Dibley, Toongabbie, Victoria (AU); Emanuele Viterbo, Clayton, Victoria (AU); Viduranga Wijekoon, Clayton, Victoria (AU)

(21) Appl. No.: 18/030,464

(22) PCT Filed: Oct. 6, 2021
(86) PCT No.: PCT/AU2021/051162

§ 371 Date: Apr. 5, 2023

(30) Foreign Application Priority Data
Oct. 6, 2020 (AU) 2020903611

(51) Int. Cl. G06Q 30/018; G16B 40/10; C12Q 1/6869; C07H 21/04

(52) U.S. Cl. CPC G06Q 30/018; G16B 40/10; C12Q 1/6869; C07H 21/04; G06Q 2220/10

(57) ABSTRACT

This disclosure relates to a method for creating an oligonucleotide sequence to represent digital data. A processor selects from a first set of multiple oligonucleotide sequences one oligonucleotide sequence for each of multiple parts of the data. The multiple oligonucleotide sequences are configured to generate an electric time-domain signal from one oligonucleotide sequence that is distinguishable from the electric time-domain signal from another oligonucleotide sequence. The electric time-domain signal is indicative of an electric characteristic of one or more nucleotides present in an electric sensor at any one point in time. The processor then combines the one oligonucleotide sequence for each of multiple parts of the data into a single oligonucleotide sequence that represents a single oligonucleotide molecule to encode the digital data.

### CROSS-REFERENCE TO RELATED APPLICATIONS

The present application claims priority from Australian Provisional Patent Application No 2020903611 filed on 6 Oct. 2020, the contents of which are incorporated herein by reference in their entirety.

### TECHNICAL FIELD

This disclosure relates to creating oligonucleotide sequences to represent digital data.

### BACKGROUND

Counterfeiting and piracy has increased substantially over the last two decades, with counterfeit and pirated products found in almost every country across the globe and in virtually all sectors of the economy. Estimates of the levels of counterfeiting and the value of such products vary. However, the value of global trade in counterfeit and pirated products in 2013 was estimated at $461 billion (OECD and EUIPO, 2016, Trade in Counterfeit and Pirated Goods: Mapping the Economic Impact). For example, counterfeit drugs are responsible for one million deaths and cost the industry $200 billion each year. Recent studies estimate that 10% of drugs sold each year are counterfeit, a number that is anticipated to increase with the rise of online pharmacies and 3D-printed medicines. The rapidly expanding medicinal and recreational cannabis markets are also particularly exposed to counterfeiters who may produce compositionally similar but substandard products with basic equipment.

One way to address these challenges may be by labelling products with encoded DNA tags. However, this often requires raw signal data to be first base-called into DNA code, i.e. A, C, G, T. The conversion of raw signal data to base-called data is computationally expensive and not compatible for laptop and smart phone sequencing devices such as the Oxford Nanopore MinION or SmidgION.

### SUMMARY

A method for creating an oligonucleotide sequence to represent digital data comprises:
selecting from a first set of multiple oligonucleotide sequences one oligonucleotide sequence for each of multiple parts of the data, the multiple oligonucleotide sequences being configured to generate an electric time-domain signal from one oligonucleotide sequence that is distinguishable from the electric time-domain signal from another oligonucleotide sequence, the electric time-domain signal being indicative of an electric characteristic of one or more nucleotides present in an electric sensor at any one point in time; and
combining the one oligonucleotide sequence for each of multiple parts of the data into a single oligonucleotide sequence that represents a single oligonucleotide molecule to encode the digital data.

The electric sensor may comprise a nanopore.

The method may further comprise determining the first set by selecting the multiple oligonucleotide sequences from multiple candidate sequences.

Selecting the multiple oligonucleotide sequences from multiple candidate sequences may be based on a distance between a first candidate sequence and a second candidate sequence. Determining the first set may comprise calculating the distance between a first simulated electric time-domain signal from the first candidate sequence and a second simulated electric time-domain signal from the second candidate sequence. Calculating the distance may comprise calculating an error of matching the first simulated electric time-domain signal to the second simulated electric time-domain signal subject to a time domain transformation that minimises the error. Calculating the distance may be based on dynamic time warping or correlation optimised warping.

Determining the first set may comprise performing a Trellis search across different combinations of nucleotides.

The method may further comprise inserting a spacer sequence between each two of the multiple oligonucleotide sequences. The spacer sequence may be of sufficient length to generate, for a second oligonucleotide sequence from the first set, a predictable interference from the spacer sequence and not a preceding first oligonucleotide sequence.

The one or more nucleotides present in the electric sensor at any one point in time may comprise a number f of nucleotides present in the electric sensor at any one point in time, and the spacer sequence may be of length kₛ with f≤kₛ≤2f.

The spacer sequence may comprise one or more of:
A homopolymer comprised of one of the set {A} or {T}
An alternating copolymer comprised of two species of alternating monomeric nucleotides {A, T} or {A, C} or {A, G}
An alternating copolymer comprised of two species of alternating dimeric nucleotides {AA, TT} or {AA, CC} or {AA, GG}
An alternating copolymer comprised of three species of alternating trimeric nucleotides {AAA, TTT} or {AAA, CCC} or {AAA, GGG}
An alternating copolymer comprised of four species of alternating tetrameric nucleotides {AAAA, TTTT} or {AAAA, CCCC} or {AAAA, GGGG}
A sequence containing one or more repeats of {AAAG} and/or {AAG}
A sequence containing one or more repeats of {TGA}
A sequence containing one or more Artificially Expanded Genetic Information System (AEGIS) nucleotides of the set {Z, P, S, B}

The method may further comprise selecting the spacer sequence from a second set of spacer sequences comprising more than one spacer sequences to encode further digital data.

The method may further comprise repeating the method to create more than one oligonucleotide molecules comprising spacer sequences between oligonucleotide sequences, the spacer sequences being selected to create an index between the more than one oligonucleotide molecules.

The method may further comprise repeating the method to create more than one oligonucleotide molecules comprising spacer sequences between oligonucleotide sequences, the spacer sequences being selected to obfuscate data encoded in the more than one oligonucleotide molecules.

The method may further comprise decoding the digital data from the single oligonucleotide molecule. Decoding may comprise capturing an electrical time-domain signal indicative of an electric characteristic of one or more nucleotides present in an electric sensor at any one point in time as the single oligonucleotide molecule passes through the sensor; and identifying the multiple oligonucleotide sequences from the first set in the captured electrical time-domain signal.

Identifying the multiple oligonucleotide sequences from the first set may comprise matching the captured electrical time-domain signal against simulated electrical time-domain signals associated with the multiple oligonucleotide sequences in the first set.

Decoding may further comprise:
identifying spacer sequences in the captured electrical time-domain signal;
splitting the captured electrical time-domain signal where the identified spacer sequences are identified;
identifying one of the multiple oligonucleotide sequences of the first set for each split.

Decoding may be based on dynamic time warping or correlation optimised warping between each split and the multiple oligonucleotide sequences in the first set.

The method may further comprise synthesising the molecule; and adding the molecule to a product for verification of the product.

Verification of the product may comprise decoding the digital data from the molecule; and performing an cryptographic operation in relation to the digital data and verify the product based on verification data.

Software, when executed by a computer, causes the computer to perform the above method.

A computer system for creating an oligonucleotide sequence to represent digital data comprises:
data memory to store a first set of multiple oligonucleotide sequences; and
a processor configured to:
   select from the first set of multiple oligonucleotide sequences one oligonucleotide sequence for each of multiple parts of the data, the multiple oligonucleotide sequences being configured to generate an electric time-domain signal from one oligonucleotide sequence that is distinguishable from the electric time-domain signal from another oligonucleotide sequence, the electric time-domain signal being indicative of an electric characteristic of one or more nucleotides present in an electric sensor at any one point in time; and
   combine the one oligonucleotide sequence for each of multiple parts of the data into a single oligonucleotide sequence that represents a single oligonucleotide molecule to encode the digital data.

An oligonucleotide molecule represents digital data, wherein the molecule comprises multiple oligonucleotide sequences combined into the molecule, wherein the multiple oligonucleotide sequences are configured to generate an electric time-domain signal from one oligonucleotide sequence that is distinguishable from the electric time-domain signal from another oligonucleotide sequence, the electric time-domain signal being indicative of an electric characteristic of one or more nucleotides present in an electric sensor at any one point in time.

The multiple oligonucleotide sequences combined into the molecule include two or more of the sequences provided in one of the following sets of nucleotide sequences:
a) SEQ ID NOs: 1 to 16;
b) SEQ ID NOs: 17 to 32;
c) SEQ ID NOs: 33 to 96;
d) SEQ ID NOs: 97 to 160;
e) SEQ ID NOs: 161 to 416; or
f) SEQ ID NOs: 417 to 672.

A kit for verifying a product's identity comprises one or more of the above oligonucleotide molecules.

A method for manufacturing an identifiable product comprises:
manufacturing the product;
selecting from a first set of multiple oligonucleotide sequences one oligonucleotide sequence for each of multiple parts of digital identification data, the multiple oligonucleotide sequences being configured to generate an electric time-domain signal from one oligonucleotide sequence that is distinguishable from the electric time-domain signal from another oligonucleotide sequence, the electric time-domain signal being indicative of an electric characteristic of one or more nucleotides present in an electric sensor at any one point in time; and
combining the one oligonucleotide sequence for each of multiple parts of the data into a single oligonucleotide sequence that represents a single oligonucleotide molecule to encode the digital identification data;
synthesising the oligonucleotide molecule; and
adding the synthesised oligonucleotide sequence to the product to allow decoding the digital identification data to verify the product's identity.

The method may further comprise:
calculating a first hash value of digital identification data, the first hash value being associated with the product; and
comparing a second hash value of the decoded digital identification data to the first hash value to verify the product's identity.

A method of verifying a product's identity, the method comprising:
providing a product to which a oligonucleotide molecule has been added,
obtaining an electrical signal indicative of a sequence of the oligonucleotide molecule;
selecting from a first set of multiple oligonucleotide sequences one oligonucleotide sequence for each of multiple parts of the electrical signal, the multiple oligonucleotide sequences being configured to generate an electric time-domain signal from one oligonucleotide sequence that is distinguishable from the electric time-domain signal from another oligonucleotide sequence, the electric time-domain signal being indicative of an electric characteristic of one or more nucleotides present in an electric sensor at any one point in time; and
decoding digital data encoded by the multiple oligonucleotide sequences to verify the product's identity based on the decoded digital data.

The method may further comprise determining a hash value of the decoded digital data, and comparing the hash value to a predetermined value for the product to verify the product's identity.

An identifiable product comprises:
one or more product constituents; and
a synthesised oligonucleotide molecule added to the one or more product constituents, wherein
the synthesised oligonucleotide molecule is represented by a single oligonucleotide sequence,
the single oligonucleotide sequence is a combination of oligonucleotide sequences comprising one oligonucleotide sequence selected for each of multiple parts of digital data from a first set of multiple oligonucleotide sequences to encode the digital data,
the multiple oligonucleotide sequences being configured to generate an electric time-domain signal from one oligonucleotide sequence that is distinguishable from the electric time-domain signal from another oligonucleotide sequence, the electric time-domain signal being indicative of an electric characteristic of one or more nucleotides present in an electric sensor at any one point in time; and
the digital data allows verification of the product's identity from decoding the digital data from the synthesised oligonucleotide molecule.

The digital data may be associated with a first hash value and the first hash value allows comparing a second hash value of a result from decoding the digital data to the first hash value to verify the product's identity.

The product may further comprise a package containing the product, wherein the first hash value is incorporated onto the package.

In the above method, the above software, the above computer system, the above oligonucleotide molecule, the above kit, or the above identifiable product, the first set of multiple oligonucleotide sequences consists of:
a) SEQ ID NOs: 1 to 16;
b) SEQ ID NOs: 17 to 32;
c) SEQ ID NOs: 33 to 96;
d) SEQ ID NOs: 97 to 160;
e) SEQ ID NOs: 161 to 416; or
f) SEQ ID NOs: 417 to 672.

Optional features disclosed in relation to one of the aspects of method, computer system, molecule, product, software and others, are equally optional features to the other aspects.

### BRIEF DESCRIPTION OF DRAWINGS

FIG. 1 illustrates a sequencing system 100 comprising an electric nanopore sensor.

FIG. 2 illustrates a method 200 for creating an oligonucleotide sequence that represents digital data.

FIG. 3 Example of an oligonucleotide strand comprised of data symbols from the alphabet A_{D}. Here, 301 is a codeword that is comprised of 302 n data symbol sequences from the alphabet A_{D}. Alphabet A_{D} may be of any size |A_{D}|. The 301 codeword is flanked by a 303 forward primer site and 304 reverse primer site.

FIG. 4 illustrates an example of an oligonucleotide strand comprised of data symbols from the alphabet A_{D} and spacer symbols from another alphabet set A_{S}. In this example, 401 is a codeword that is comprised of two different alphabets of alternating symbol sequences, 402 and 403. Symbols from the set A_{D} 402 encode information, whilst symbols from the set A_{S} encode information (if |A_{S}|>1) and additionally perform the function of spacer symbols. Due to the additional constraints on A_{S} symbols, in general |A_{S}|<|A_{D}|. The advantage of this approach is that the spacer sequences encode some data, thereby increasing the rate r (in bits base⁻¹). A_{D} symbol sequences are selected so that each symbol signature, dᵢ(t), is at a defined minimum mutual Dynamic Time Warping (DTW) or Correlation Optimised Warping (COW) cost distance. The 501 codeword is flanked by a 504 forward primer site and 505 reverse primer site.

FIG. 5 illustrates an example of a multi-strand ID tag where information is distributed across multiple oligonucleotide strands. In this example, two alphabets are once again used to encode information into an ‘alternating codeword’ comprised of symbols from the alphabet A_{D} and A_{S} (See also FIGS. 4 and 5). Here, 601 is a multi-strand ID tag comprised of a total of L strands, where each strand encodes a codeword that is comprised of n 602 data symbols that are separated by n+1 spacer symbols. 603 data symbols from the set A_{D} encode information, whilst 604 spacer symbols from the set A_{S} encode index information about the location of a codeword in a multi-strand ID tag. Due to the additional constraints on A_{S} symbols, in general |A_{S}|<|A_{D}|. In this example |A_{D}|=256 and |A_{S}|=2 and L<=2ⁿ⁺¹≤32 possible indexes that determine the location of a strand in a multi-strand ID tag (note that all possible indexes are not required to be used). The advantage of this approach is that the index encoded into the spacers permit information to be distributed across multiple strands in a ID tag, thereby permitting a single ID tag to be encoded into more than a single DNA strand. A_{D} symbol sequences are selected so that each symbol signature, dᵢ(t), is at a defined minimum mutual Dynamic Time Warping (DTW) or Correlation Optimised Warping (COW) cost distance. Each 602 codeword is flanked by a 605 forward primer site and 606 reverse primer site.

FIG. 6 illustrates simulated codeword signals showing data symbols from the alphabet A_{D} (long, 701) and spacer symbols from the alphabet A_{S} (short, 702). The x-axis units are time (˜4000 Hz, 1/4000 s) and the y-axis units are analogue current output (normalised).

FIG. 7 illustrates error probabilities of template and complementary current signatures of data symbols from an alphabet of size 16 where k_{D}=12.

FIG. 8 illustrates error probabilities of template and complementary current signatures of data symbols from an alphabet of size 64 where k_{D}=12.

FIG. 9 illustrates an alphabet of 16 data symbols A_{D} together with simulated analogue symbol signatures dᵢ(t), selected with absolute DTW cost distance. The x-axis units are time (˜4000 Hz, 1/4000 s) and the y-axis units are analogue current output (normalised).

FIG. 10A illustrates an alphabet of 16 data symbols A_{D} together with analogue symbol signatures dᵢ(t), selected with Euclidean DTW cost distance. The x-axis units are time (˜4000 Hz, 1/4000 s) and the y-axis units are analogue current output (normalised).

FIG. 10B illustrates a histogram of the pair-wise DTW cost and pair-wise Hamming distance of the alphabet in FIG. 10A.

FIG. 11A illustrates eight example simulated symbols from an alphabet of 64 data symbols A_{D} together with analogue symbol signatures dᵢ(t), selected with absolute DTW cost distance. The x-axis units are time (˜4000 Hz, 1/4000 s) and the y-axis units are analogue current output (normalised).

FIG. 11B illustrates a histogram of the pair-wise DTW cost and pair-wise Hamming distance of the alphabet in FIG. 11A.

FIG. 12A illustrates eight example symbols from an alphabet of 64 data symbols A_{D} together with analogue symbol signatures dᵢ(t), selected with Euclidean DTW cost distance. The x-axis units are time (˜4000 Hz, 1/4000 s) and the y-axis units are analogue current output (normalised).

FIG. 12B illustrates histograms of pair-wise DTW cost and pair-wise Hamming distance of the all the 64 data symbols of the alphabet referred to above in relation to FIG. 12A.

FIG. 13A illustrates eight example symbols from an alphabet of 256 data symbols A_{D} together with analogue symbol signatures dᵢ(t), selected with absolute DTW cost distance. The x-axis units are time (˜4000 Hz, 1/4000 s) and the y-axis units are analogue current output (normalised).

FIG. 13B illustrates histograms of pair-wise DTW cost and pair-wise Hamming distance of the all the 64 data symbols of the alphabet referred to above in relation to FIG. 13A.

FIG. 14A illustrates eight example symbols from an alphabet of 256 data symbols A_{D} together with analogue symbol signatures dᵢ(t), selected with Euclidean DTW cost distance. The x-axis units are time (˜4000 Hz, 1/4000 s) and the y-axis units are analogue current output (normalised).

FIG. 14B illustrates histograms of pair-wise DTW cost and pair-wise Hamming distance of the all the 256 data symbols of the alphabet referred to above in relation to FIG. 14A.

FIG. 15 illustrates examples of SDSDSDSDS ID tags that include spacers symbols S that encode data. In this example A_{S}={S₁, S₂}→{0, 1}→{TTTTTTTT, AGAGAGAG}. Spacer configurations, C_{S}, are given in the title of each figure panel and shown in red in the analogue data. The x-axis units are time (˜4000 Hz, 1/4000 s) and the y-axis units are analogue current output (normalised).

FIG. 16 illustrates examples showing real nanopore data of five different SDSDSDSDS ID tags. In these figures, the blue dots are the raw analogue current signatures (normalised) and the red lines identify spacer symbols from A_{S} that flank data symbols from A_{D}. The x-axis units are time (˜4000 Hz, 1/4000 s) and the y-axis units are analogue current output (normalised).

FIG. 17 (A-D) shows real nanopore output of sequences containing AEGIS bases of the set {Z, P, B, S}. Panels (Ai)-(Di) show average raw nanopore output for tags ID_AG_1-4 amplified in the presence of dNTPs only {A, C, G, T}. Panels (Aii)-(Dii) show average raw nanopore output for tags ID_AG_1-4 amplified in the presence of dNTPs {A, C, G, T, Z, P, B, S}. The actual sequences are given above each panel, where N may be one of {A, C, G, T}. The x-axis units are time (˜4000 Hz, 1/4000 s) and the y-axis units are analogue current output (normalised).

FIG. 18 is an overview of decoding nanopore signals. First step of decoding is to normalise the nanopore signal. Then, spacer detection program is run with the normalised signal. The program may not be able to locate the required number of spacers, in which case, the signal will be rejected. If the required number of spacers are found, then the in-between signal sections are extracted, which are the ‘received’ data symbols. This set of received symbols then undergo a two-step decoding process; first they are decoded with the signatures of template sequences in the data alphabet, and after that with the signatures of reverse complementary sequences. Each decoding step generates the likeliest codeword, which has a certain cost. The final estimate is the sequence with the least cost of the two. current output (normalised).

FIG. 19 is an overview of spacer detection in decoding. Spacer detection program outlined in the flowchart is when all the spacers are of the same type, and generate an almost flat signature. The input to the program is the normalised nanopore signal. The program first finds the sections which are almost flat. Out of these, first those in a significantly different amplitude region than the rest (the outliers) are rejected. Then, sections which are placed very close to each other in the signal are combined, assuming the in-between high-amplitude signal is due to measurement noise. Another outlier removal step is then carried out. Finally, there could be more than the required number of spacer regions (represented with N here) detected. Then, the N adjacent regions which have sufficiently long gaps (this depends on the value of k_{D}) are chosen as the spacer regions.

FIG. 20 illustrates identifying flat regions in a nanopore signal. A flat region is determined from the amplitude differences between samples of the region. For each sample in the signal, the amplitude difference with the mean of the on-going section is computed. If this is less than the allowed difference (MAX_DIFF), sample is added to the section and section mean is updated. In the case a section is not going on, amplitude of the sample is used as the section mean for the next sample. If the difference is larger than allowed, it is checked if the maximum number of allowed noisy samples is reached. If not, the sample is added to the section, and the number of noisy samples is incremented. If this number has already been reached, the sample would not be added to the section, and it would mark the end of the ongoing section. It is then checked if this section is long enough, and whether the mean amplitude is within the allowed range. If both requirements are satisfied, the section is added to the initial estimates of spacer regions. Algorithm would then move on to the next sample in the signal. There are a few parameters in the algorithm that the user have to set to values suitable to the particular application. These are MAX_DIFF: Maximum difference between the amplitude of a sample, and the ongoing flat region's mean amplitude, for the sample to be added to the region. Also used to check whether the mean amplitude difference between two different flat regions is significant. MIN_LEN: Minimum required length for a flat region. MAX_NOISE: Maximum number of noisy (sample amplitude significantly different to the mean) samples allowed per flat region. MIN_PLD_LEN: Minimum required length for a symbol signature (payload region). N: Number of spacer required.

FIG. 21 illustrates removing spacer outliers. Outliers in the initial estimates for spacer regions are decided based on the mean amplitudes. For each estimate, mean difference with all other estimates are computed. If for more than 50%, the mean difference is >MAX_DIFF, the position is marked as an outlier. After considering each initial estimate, all estimates marked as outliers are removed from the set. There are a few parameters in the algorithm that the user may have to set to values suitable to the particular application. These are MAX_DIFF: Maximum difference between the amplitude of a sample, and the ongoing flat region's mean amplitude, for the sample to be added to the region. Also used to check whether the mean amplitude difference between two different flat regions is significant. MIN_LEN: Minimum required length for a flat region. MAX_NOISE: Maximum number of noisy (sample amplitude significantly different to the mean) samples allowed per flat region. MIN_PLD_LEN: Minimum required length for a symbol signature (payload region). N: Number of spacer required.

FIG. 22 illustrates combining close flat regions. The gap between any two spacer regions should be large enough for the signature of a length k_{D} sequence. Minimum possible gap, MIN_PLD_LEN, depends on the value of k_{D}. For each estimate for a spacer region, the gap to the next region is compared with MIN_PLD_LEN, and if the gap is smaller, then the two sections are combined. This is done repeatedly for the set of estimates until no two sections are combined. There are a few parameters in the algorithm that the user have to set to values suitable to the particular application. These are MAX_DIFF: Maximum difference between the amplitude of a sample, and the ongoing flat region's mean amplitude, for the sample to be added to the region. This is also used to check whether the mean amplitude difference between two different flat regions is significant. MIN_LEN: Minimum required length for a flat region. MAX_NOISE: Maximum number of noisy (sample amplitude significantly different to the mean) samples allowed per flat region. MIN_PLD_LEN: Minimum required length for a symbol signature (payload region). N: Number of spacer required.

### DESCRIPTION OF EMBODIMENTS

### Glossary

A_{D}—Set of data symbols forming a data alphabet of size |A_{D}|
Alphabet—The set of symbols used to encode data. This set may be mapped to any structure traditionally used to represent data, such as a finite field. In this case, each element of the field will be represented with a symbol in the alphabet.
A_{S}—Set of spacer symbols forming a spacer alphabet of size |A_{S}|
AEGIS base—one of the set of nucleotide {Z, P, B, S}
B—the AEGIS nucleotide 6-amino-9[(1′-ß-D-2′-deoxyribofiiranosyl)-4-hydroxy-5-(hydroxymethyl)-oxolan-2-yl]-1H-purin-2-one
b—Number of bases in a strand
Base—A nucleotide of the set {A, C, G, T, U, Z, P, B, S}
C—A codeword that includes data and optionally spacer symbols
Codeword—an oligonucleotide strand that include data symbols and optionally spacer symbols
COW—Correlation Optimised Warping C_{D}— The configuration of data symbols in an ID tag
C_{S}—The configuration of spacer symbols in an ID tag
Data symbol (D)—An oligonucleotide sequence used to represent a data symbol of the encoding alphabet. Signature of a data symbol is represented with d(t).
Dᵢ—i′th data symbol (i=1, . . . , |A_{D}|) of the (data) alphabet. Signature represented with dᵢ(t).
dNTPs—deoxynucleotides of the set {A, C, G, T}
dsDNA—A double stranded oligonucleotide comprised of one or more of A, C, G, T, U, Z, P, B, S
DTW—Dynamic Time Warping
dXTPs—deoxynucleotides of the set {A, C, G, T, U, Z, P, B, S}
f—The number of bases inside a nanopore at any one time
ID tag or tag—A DNA sequence of the form SDSDSD . . . SDS, flanked with primers. When manufactured, could be composed of either one or more oligonucleotide strands in either single-stranded or double-stranded form.
k_{D}—Number of bases forming a data symbol
k_{S}—Number of bases forming a spacer symbol
L—Number of strands in one multi-strand ID tag
mer—Abbreviation of oligomer, a string of nucleotides, e.g. an 8 mer is a strand of 8 nucleotides
multi-strand—Set of strands containing a single, manufactured ID tag
N—Number of data sequences per ID tag (N=nL)
n—Number of data sequences per strand. In the case of a multi-strand, each individual strand would have the same number of data sequences (same ‘n’).
nt—A nucleotide, either free or in a strand of nucleotides (i.e. an oligomer or ‘mer’)
Nucleotide—A natural base of the set {A, C, G, T, U} or AEGIS base of set (Z, P, B, S)
Oligonucleotide sequence—A sequence of bases or nucleotides,
Oligonucleotide strand—A polymer of bases or nucleotides, also referred to as a ‘fragment’
P—the AEGIS nucleotide 2-amino-8-(1′-b-D-2′-deoxyribofuranosyl)-imidazo-[1,2a]-1,3,5-triazin-[8H]-4-one
r—Number of bits encoded per base before any outer code is applied. When using an outer code to improve error correction, r would be referred to as ‘inner code rate’.
R—Rate of the outer code, in the number of ‘information’ bits encoded per base.
Signature—The analogue signal generated by a DNA sequencing machine
S—the AEGIS nucleotide 3-methyl-6-amino-5-(1′-b-D-2′-deoxyribofuranosyl)-pyrimidin-2-one. Note: may also refer to a spacer symbol.
Sⱼ-j′th (j=1, . . . , |A_{S}|) spacer symbol of the (spacer) alphabet. Signature is sⱼ(t).
Spacer symbol (S)—A oligonucleotide sequence used to separate two data sequences. The corresponding signature is represented with s(t).
ssDNA—A single stranded oligonucleotide comprised of one or more of A, C, G, T, U, Z, P, B, S.
Symbol—An oligonucleotide sequence used to represent some element of the alphabet set used to encode data. Any encoded data will be a concatenation of these symbols.
Z—the AEGIS nucleotide 6-amino-3-(1′-b-D-2′-deoxyribofuranosyl)-5-nitro-1H-pyridin-2-one

### Supply Chain Integrity

As set out above, there is a need for methods and systems against counterfeiting and piracy. One solution is to add oligonucleotides to products, components, constituents of mixtures etc. Information encoded into these oligonucleotides can be used to verify the producer of the product. More particularly, the producer generates digital data, such as a secret based on cryptographic algorithms including hash or encryption algorithms. The digital data is then encoded into a oligonucleotide sequence and a corresponding molecule is synthesised and added to the product. A customer, receiver or processor of the product can extract the molecule and decode the digital data encoded thereon. The customer, receiver or processor can then verify the product, such as by performing corresponding cryptographic algorithms and comparing the result to the decoded digital data.

In one example of addressing challenges to supply chain monitoring, an alphanumeric identifier may be encoded into a synthetic oligonucleotide using the approaches disclosed herein. Either the alphanumeric codeword, or the oligonucleotide sequence, or a combination of both, or a combination of both plus some padding text, may be passed through an encryption algorithm that generates a hash value. Because hash functions are deterministic and computationally infeasible to reverse engineer, the alphanumeric hash value of the oligonucleotide may be displayed publicly on a package, for example, as a string of alphanumeric characters or as a data matrix or QR code. The encoded oligonucleotide is added (mixed in or affixed to) a product or ingredient, thereby giving the product or ingredient a unique oligonucleotide ‘fingerprint’. The hash value representation of the oligonucleotide in the product or ingredient may be displayed on the product packaging, thereby creating an immutable link between the product and packaging.

This approach may also be used for multiple ingredients in a product, where each unique ingredient hash value is concatenated together and hashed again to form a binary tree of hashes (analogous to block chain). At the point where a final product is made or assembled, the final product batch hash value is a representation of all of the ingredient hash values in the final product. If desired, the batch hash value may then be hashed with a counter or time stamp to generate a unique hash value for individual packages from the same batch. The resulting unique package hash value may be considered analogous to a serial number, but with the security advantage that the package hash value (displayed as a QR or data matrix code) is immutably linked to ingredients in the product, rather than being an arbitrary number. The unpackaged product may be verified by recovering, sequencing, decoding, and hashing the oligonucleotide tags in the product, and either looking up product information associated with the resulting hash value/s in a database, or cross-validating the oligonucleotide derived hash value/s with the package hash value. Further examples can be found in PCT publication WO 2020/028955 entitled “SYSTEMS AND METHODS FOR IDENTIFYING A PRODUCTS IDENTITY”, which is incorporated herein by reference.

In one example, the hash argument may comprise a product code or manufacturing code or simply a random number that is not associated with any particular identifying functionality. A computer calculates a first hash value of the hash argument. The hash value is calculated by a hash function which can take a range of different forms depending on the security requirements of the overall system. For example, a hash value may be calculated by multiplicative hashing where the overall number of different sequences is limited and therefore collision is unlikely. In other examples, more sophisticated functions, such as MD5 or preferably, SHA-2 or SHA-3 can be used. Since these sophisticated functions are highly optimised, the computational burden is minimal and therefore, there is little downside to using a hash function that is more sophisticated than required by this particular application.

After, before, or during calculating the hash value, the oligonucleotide sequence is determined to encode the hash argument, that is, the plain text before hashing. The sequence is then used to synthesise a molecule using known techniques and added to the product. This may involve mixing the synthesised (chemical form) of the molecule into the product. The product may then pass through a supply chain to reach a recipient, such as the end customer or an intermediate manufacturer or quality control agent.

It is now desired that the recipient can verify the identity of the product. Therefore, the recipient sequences a second oligonucleotide sequence from the product, where it is unknown whether that sequence is the same as the sequence of the molecule added by the original (or ‘upstream’) manufacturer. To verify this, the intermediary can decode digital data encoded in the molecule and calculate a second hash value of the sequenced molecule and compare 107 the second hash value to the first hash value to verify the product's identity. If the second hash value is identical to the first hash value, the product's identity is verified. If the hashes are different, the product's identity is not verified.

The hash value may also be calculated based on additional data that may be a product identifier, entity identifier of the handling entity at that point, shared secret, public key, time stamp, counter, or product-unique product identifier that is unique to that particular individual “instance” of the product. This additional data may either be concatenated with the oligonucleotide sequence before the hash is calculated or the hash of the oligonucleotide sequence may be concatenated with the additional information and another hash calculated on the result. The important aspect is that any minor chance in the additional data leads to a completely different hash and it is practically impossible to change the additional data such that the hash stays the same or to determine the additional data from the hash alone.

A package identification technology (PI) is any technology that is displayed on a package for the purpose of identifying a product. Package identification technologies may include, but are not limited to: inks, dyes, holograms, bar codes, QR codes, RFID, silicon dioxide encoded particles, product spectral image data, and IoT devices. The PI may display a hash value at any node of a manufacturing process or supply chain.

The use of hashing functions permits a safe and secure link between the molecule tags in the product, and the product packaging.
PI is displayed publicly on the package
H(digital data) provides a cryptographic link to the digital data, whilst keeping the digital data secret.
PI incorporates the hash of the digital data that is encoded by the molecule in a product.
The PI code may be a genesis hash, the most recent node hash at packaging, or any other node hash in a product's hash chain/tree.
The PI may be an alternative identifier that points to a node hash value.

### Examples of Practical Use Cases for the Disclosed Technology

Palm oil. Palm oil is used is a wide range of products including food products, cosmetics, cleaning products and pharmaceuticals. Palm oil production is also linked to deforestation, biodiversity loss and poor work conditions. The disclosed technology may be integrated with existing certification schemes (for e.g RSPO) so that the origin of palm oil can be traced back to a sustainably certified manufacturer from the end product alone.

Pharmaceuticals. Counterfeit pharmaceuticals are responsible for one million deaths and cost the industry $100B each year. Incidents of drug counterfeiting are increasing with the rise of online pharmacies. Additionally, in many developing and transition economies, medications are sold as unpackaged individual tablets or doses. The capacity to recover supply chain information from an individual tablet alone could address the massive human and economic cost of fake pharmaceuticals.

Cannabis products. The cosmetic and medicinal cannabis industry is highly exposed to counterfeiting from backyard and recreational growers. Fake products present serious concerns as the active compound content in cannabis (THC, CBD) may vary widely in plants that are grown under different conditions and across different plant strains. Fake medicinal products that have not be subjected to stringent quality control steps, and contain sub-therapeutic cannabinoid levels, may lack therapeutic efficacy. Additionally, in some countries such as the USA, products must be grown, manufactured, and sold within state boundaries for tax purposes. The ease with which products may cross state boundaries could result in the loss in billions of dollars in tax revenue. The disclosed invention offers a means to track material from the ‘plant to product’, as well as mark various mixing and quality control steps along the manufacturing/supply chain. This information can be recovered from the unpackaged end product alone, and thereby address the problems highlighted above.

Illicit drug precursors (e.g. methamphetamine). The disclosed technology may be used to traceback the chain of custody of products that are misused. For example, legal ingredients used as precursors for the manufacture of illicit drugs, such as methamphetamine, may be traced to the last legitimate node in a supply chain from a drug sample alone. This capability may be useful for pinpointing fraudulent or leaking nodes in a supply chain, and gathering intelligence on how narcotics networks operate.

Kosher and Halal. Kosher and Halal products cannot be identified by the end product alone (there is no test of Kosher and Halal). The disclosed technology may be used to verify and track products from certified Kosher and Halal producers, and thereby address widespread counterfeiting problems in the industry.

Milk products. Counterfeit milk products are frequently detected in Asian markets, and have resulted in the hospitalisation of more than 50,000 infants from melamine poisoning since 2008. The capacity to recover and verify all supply chain information, from the milk product alone, could address this problem.

Ammunition. Recent advances in firearms technology have exacerbated the already difficult task of detecting illicit arms and ammunition transfers. In 2012, firearms were responsible for 41% of non-conflict homicides worldwide, with approximately 57% of these incidents remaining unsolved. In 2016, President Obama and the American Medical Association declared gun violence a public health concern, which is estimated to cost the US economy $229 billion each year—even more than the cost of obesity. The advent of modular, polymer, and 3D printed guns have also brought new challenges for firearms tracing and registration. The capacity to label and trace oligonucleotide tagged ammunition to the bullet entry wound has been demonstrated previously. The innovation disclosed offers a way to trace and trace crime via labelled ammunition.

Other applications. The disclosed technology may be used to track and trace many other products including, but not limited to: wine, cosmetics, precious stones, chemicals, fertilizers, bank notes, casino chips, and luxury items.

### Nanopore Sequencing

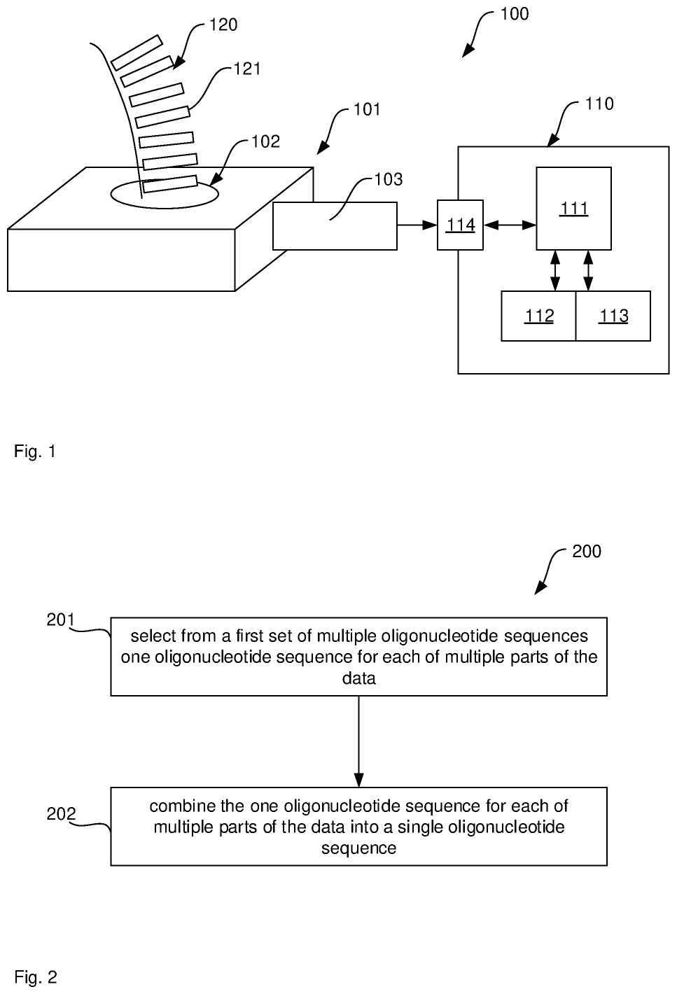

FIG. 1 illustrates a sequencing system 100 comprising an electric Nanopore sensor 101 with a nano-meter pore 102 and read-out electronics 103. Sensor 101 is connected to a computer system 110, comprising a processor 111, program memory 112, data memory 113 and a communication port 114. Many different variations of computer system 110 can be used including personal computers (PCs), mobile computers (Laptops), smart phones, cloud computing environments etc. In one example, the sensor 101 is connected to computer system 110 via a universal serial bus (USB). Other connections are of course possible.

It is noted that some examples herein relate to the use of DNA but it is noted that other types of oligonucleotide sequences, such as RNA or DNA/RNA hybrid with five different nucleotides or bases can be used to represent digital data.

In Nanopore sequencing as in FIG. 1, a DNA strand 120 is passed through the nano-meter size pore 102 immersed in an electrolytic solution. The DNA string 120 is a single molecule comprising a sequence of nucleotides represented as rectangles, such as nucleotide 121. Read-out electronics 103 apply a constant voltage across the pore 102, and measure the current level. Fluctuations in this current signal are due to characteristics of the DNA string 120 passing through the pore 102. Analysis of these current fluctuations enables identification of the base sequence in the string. This process, referred to as ‘basecalling’, is still not sufficiently reliable and computationally efficient to permit the broadscale use of Nanopore devices in all diagnostic applications. It is noted that instead of current signals, voltage signals may equally be useable. The signal from the read-out electronics is referred to as a time-domain electrical signal, which means that the signal comprises a series of amplitude values (representing voltage, current or other measured values). There is one amplitude value for each point in time, which makes this signal a time-domain signal. In some examples, read-out electronics 103 creates the time-domain electrical signal in the form of digital data, such as a series of bits, where a predefined number of bits encodes an intensity value and a time value. In other examples, read-out electronics 103 create the time-domain in the form of analogue data as a continuous voltage signal, for example.

The f bases inside the pore at a given time is the ‘state’ of the pore, and each state should produce a unique current level. Even the durations of these levels should be state-dependent. What makes basecalling that much more difficult is the level and duration of the current being affected by a number of factors other than the state, such as base stacking in the pore or the upstream functioning of the motor protein (for e.g.). The effects of these factors, and even all factors that can have an effect, are not completely known. Thus, the current signal can sometimes look quite ‘random’, and the signals for a particular DNA string, measured using the same device but at different times, could look quite different from one another. This stochastic nature of signals presents a significant challenge to basecalling DNA or RNA using nanopore technology.

This disclosure provides a bypass of the basecaller, and operates directly on the ‘raw’ current signal measured by the Nanopore device, which is also referred to as a ‘soft decision decoding’ system. An additional advantage of such an approach is that the current signal, or the ‘soft data’, contains more information than the ‘hard’ output of a basecaller, which can be used to increase reliability.

### Computer System

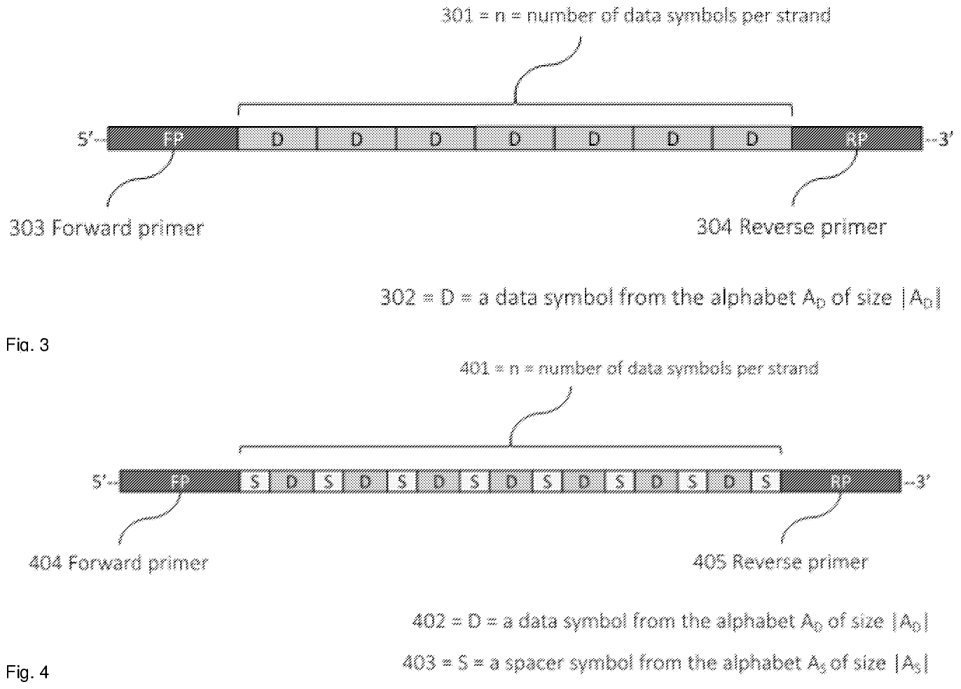

Computer receives a time-domain electric signal from read-out electronics 103 and decodes digital information that has been encoded in the DNA string 120. In that sense, processor 111 executes program code installed on non-volatile program memory 112, which causes processor 111 to perform the methods disclosed herein, such as methods for decoding data or methods for encoding data, such as method 200 in FIG. 2. It is noted that in FIG. 1, computer system 110 decodes data. Computer system 110 may also encode data to create DNA strand 120. In other examples, there are two different computer systems, one computer system for encoding data as a ‘sender’ and a second computer system decoding the data as a ‘receiver’. For example in a supply chain, the sender may be part of the manufacturing of a product, where the created DNA string is added to a product. The decoding receiver computer system is then part of the customer where the DNA string is decoded to verify the product's identity.

### Method

FIG. 2 illustrates a method 200 for creating an oligonucleotide sequence to represent digital data. It is noted here that the term “oligonucleotide sequence” refers to digital data representing or characterising a molecule. That is, an oligonucleotide sequence exists as a result of the method without any molecules being created.

When method 200 is performed by processor 111, processor 111 selects 201 from a first set of multiple oligonucleotide sequences one oligonucleotide sequence for each of multiple parts of the data. That is, there is a set of sequences (later referred to as ‘symbols’) and symbols are selected to represent parts of the data. For example, a part of the data may be a byte with 8 bits or a part of different length. The multiple oligonucleotide sequences (‘symbols’) are configured to generate an electric time-domain signal from one oligonucleotide sequence that is distinguishable from the electric time-domain signal from another oligonucleotide sequence. For example, and as detailed below, the signals may have a maximum or above-threshold distance as calculated by dynamic time warping. As set out above, the electric time-domain signal is indicative of an electric characteristic of one or more nucleotides present in an electric sensor 101 at any one point in time.

Processor combines 202 the one oligonucleotide sequence for each of multiple parts of the data, that is the selected symbols, into a single oligonucleotide sequence that represents a single oligonucleotide molecule 120 to encode the digital data.

The method may then further comprise synthesising the molecule and adding it to a product. The digital data encoded into the molecule is calculated such that it, once decoded, can be used to verify the product.

### Coding

Consider a system where data is encoded at the base-level, and a soft decoder is applied on the current signal measured. We denote the length of the DNA string after encoding with b bases. If f bases fit inside the pore at any one point in time, the current signal recorded may include up to b−f+1 different states. As the encoder is operating on bases, the decoder also requires base-level data. For a soft decoder, this means (b−f+1) probability vectors, one for each state. The i′th such vector would contain the probabilities of the i′th state being each possible set of f bases, or f-mer. Preferably, the decoder should be able to process these probability vectors and produce a reliable output.

This disclosure provides an alphabet for soft decision encoding. Each ‘letter’ of this alphabet A_{D} of size |A_{D}|, referred to as a ‘symbol’, is matched to a uniquely identifiable current signal dᵢ(t), which is produced by a short corresponding base sequence, Dᵢ. Information is represented using this ‘encoding’ alphabet, to which redundancy can also be added. For storing data, each letter is replaced with its short base sequence. Also, in-between each pair of such sequences, a short polynucleotide ‘spacer sequence’ Sᵢ is added from the alphabet A_{S} of size |A_{S}|. When the final sequence is synthesized and read by the Nanopore device, the current signal contains the signals from the encoding alphabet dᵢ(t), separated by the almost flat signals sᵢ(t) produced by the polynucleotide spacer sequences, or in some cases distinctive ‘spikey’ signals. In the examples given in this disclosure, a range of spacer sequences were tested. The decoder ‘extracted’ the signals from the alphabet and proceeded to decode information in the codeword. We refer to these extracted signals as signals ‘received’ by the decoder.

In decoding, each received signal is compared to all the reference signals in the alphabet of data symbols A_{D} and spacers A_{S}. Rather than using probabilistic approaches, the dynamic time warping (DTW) or correlation optimised warping (COW) cost between a reference signal and a received signal is used as the decoding metric. For each received signal, a vector of DTW costs is computed, and the decoder operates on these. The output of the decoder is a valid vector with the lowest overall DTW cost (computed as the sum of costs of each received signal). It should be noted that the encoding-decoding system here has no knowledge of bases; it only uses an alphabet composed of different current signatures di(t) and si(t).

Another concern in DNA data storage is the presence of the complementary strand. Single stranded sequences of DNA (ssDNA) that undergo amplification generate a complementary strand and become double-stranded DNA (dsDNA), and it is possible (about 50% of the time) that the current signal measured is for that strand. To circumvent this difficulty, this disclosure investigates multiple approaches:
1) Pre-computing the reference signals for complementary sequences as well as the template strands, and carrying out a two-step decoding process, once with references for normal sequences, and then with references for complementary ones. Outputs of both are then be compared, and the one with the lowest DTW cost metric is the final output.
2) Identifying the template and complementary strands from the 5′ primer site and from this, determining whether the template or complementary alphabet should be used for decoding, and
3) first identifying the template and complementary strands from the template and complementary spacer signatures in a query oligonucleotide strand.

In order to compute the reference signals for the short base sequences, we used the squiggle function available in ‘Scrappie’ (available from https://github.com/nanoporetech/scrappie). Using this software, it is possible to obtain an ‘average’ signal for any base sequence, which we call the ‘signature’ of the sequence. To compute the reference signals for the short base sequences some ‘training’ is performed beforehand. In one methodology for doing this, DNA sequences containing symbol sequences from A_{D} separated by spacer sequences from A_{S} are synthesized and then read using a Nanopore device. A clustering algorithm is run on the set of raw current signals. To decide the DNA sequence of each resulting cluster, a basecaller is used. Sequences that matched to the majority of signals in the basecalled cluster are taken as the sequence of that cluster. Reference signals were computed by averaging all the signals in the cluster, using DTW Barycenter Averaging.

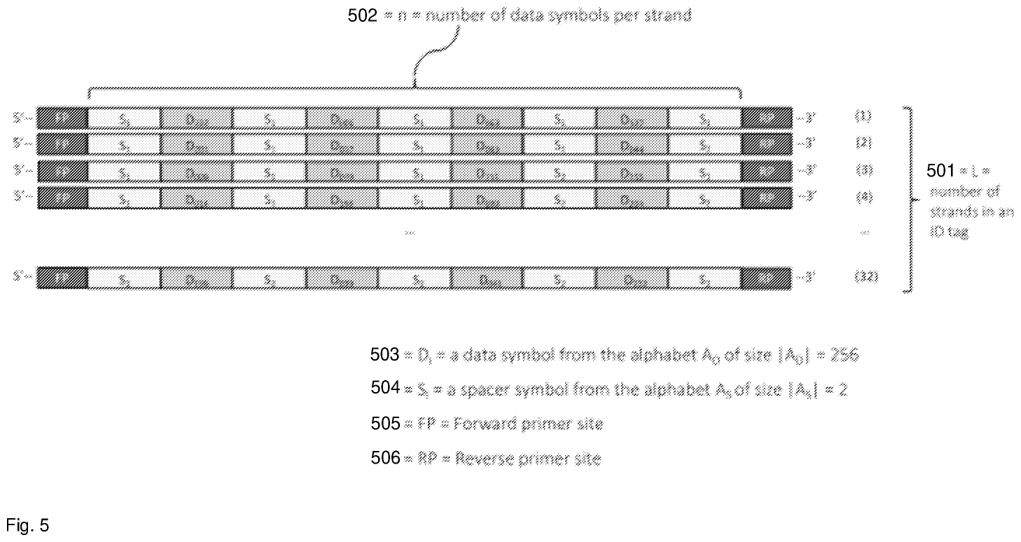

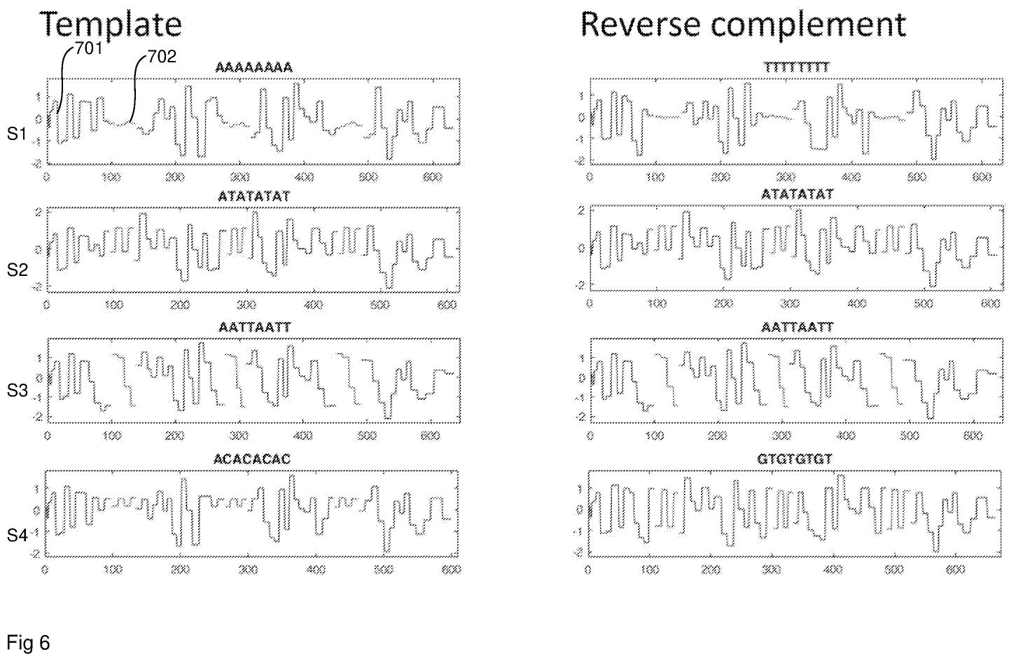

In the first iteration of the disclosed encoding system, we tested codewords that were simply constructed from a string of data symbols from the set A_{D} as shown in FIG. 3. Although this approach yielded decodable analogue output, symbol segmentation remained a challenge because the nanopore reading frame is approximately f=5-6 bases which permits 1,024-4,096 different states. Additionally, because measurements are taken in the middle of the reading frame (pore) the analogue signature produced by any oligonucleotide subsequence in an oligonucleotide strand may be affected by the 2-3 nucleotides immediately before and after the query nucleotide. Other upstream conditions, such as the function of the motor protein, upstream sequences, base stacking, etc., may also effect measurements at the pore. To address this problem, it is possible to construct codewords from alternating symbols from two different alphabets, a data alphabet A_{D} and a spacer alphabet A_{S} as shown in FIG. 4.

Data and spacer symbol selection is performed iteratively by evaluating simulated raw squiggle output, selecting candidate sequences, and generating and evaluating real output. When data alphabets A_{D} and spacer alphabets A_{S} are identified, machine learning algorithms may be applied to sequences assembled from the alphabets to aid decoding. Machine learning may be used for data decoding after spacer decoding, or it may be used for decoding both spacer and data symbols. In both cases, the neural network used for decoding should be trained with large amounts of ‘noisy’ data for which the underlying sequences/symbols are known. With the network trained sufficiently well, the raw signals generated when reading a DNA strand could be directly fed to it, and it would output the most likely sequence/symbol.

In some embodiments, it may be advantageous to perform tag decoding on spacer symbols S locally and data symbols D locally, whist in other embodiments it may be advantageous to perform tag decoding on S locally decoding on D remotely, and in yet still other embodiments it may be advantageous to perform tag decoding on S remotely and tag decoding D remotely.

### Alphabet Design (Inner Code)

The alphabet is a set of symbols constructed from k_{D} nucleotides (‘mers’). We also refer to such symbols as a letter or inner codeword. As described, in some embodiments, the ID tag is comprised of alternating letters (inner codewords) from the set A_{D} and A_{S}. Here, we disclose a methodology to select oligonucleotide inner codewords using dynamic time warping (DTW) cost as a metric, measured as either absolute distance or Euclidean distance. First, we constructed 5 sets of 500 random symbol sequences of length k_{D}=8, 10, 12, 14 and 16 nucleotides, within the following constraints:
Each data sequence of a symbol does not start with the same nucleotide as the end of the spacer sequence, or end with the same nucleotide as the start of the spacer sequence.
The maximum GC content in a symbol is ≤70%
The maximum G or C homopolymer region in a symbol is ≤3

From the 500 candidate symbols, we selected alphabets of size |A_{D}|=16, 64, 256 symbols using the absolute and Euclidean distance threshold metrics in DTW given in Table 1 and Table 2. Table 3 shows that k_{D} symbol length selection is a trade-off between the code rate (bits nt⁻¹) and minimum absolute and Euclidean distance required for reliable decoding.

TABLE 1
Absolute dynamic time warping (DTW) distance thresholds for symbol
selection of F16, F64, and F256 alphabets, where k_{D} = 12.

| | | Distance threshold |
|---|---|---|
| Alphabet | Size | (dimensionless) |
| F16abs | 16 | 59.5 |
| F64abs | 64 | 44.5 |
| F256abs | 256 | 31.5 |

TABLE 2
Euclidean dynamic time warping (DTW) distance thresholds for symbol
selection of F16, F64, and F256 alphabets, where k_{D} = 12.

| | | Distance threshold |
|---|---|---|
| Alphabet | Size | (dimensionless) |
| F16eu | 16 | 6.8 |
| F64eu | 64 | 5.375 |
| F256eu | 256 | 3.825 |

TABLE 3
Example inner code alphabet design metrics for absolute distance.

| | k_{D} = 8 | | | k_{D} = 10 | | | k_{D} = 12 | | | k_{D} = 14 | | | k_{D} = 16 | | |
|---|---|---|---|---|---|---|---|---|---|---|---|---|---|---|---|
| A | Dₘᵢₙ | D_{N} | Rᵢ | Dₘᵢₙ | D_{N} | Rᵢ | Dₘᵢₙ | D_{N} | Rᵢ | Dₘᵢₙ | D_{N} | Rᵢ | Dₘᵢₙ | D_{N} | Rᵢ |
| F16 | 40 | 5 | 0.25 | 54 | 5.4 | 0.2 | 59.5 | 4.95 | 0.167 | 71 | 5.07 | 0.143 | 83 | 5.19 | 0.125 |
| F64 | 28 | 3.5 | 0.375 | 38 | 3.8 | 0.3 | 44.5 | 3.71 | 0.25 | 55 | 3.93 | 0.214 | 65 | 4.06 | 0.188 |
| F256 | 16.75 | 2.09 | 0.5 | 25 | 2.5 | 0.4 | 31.5 | 2.63 | 0.33 | 44 | 2.86 | 0.286 | 48.5 | 3.03 | 0.25 |

Dₘᵢₙ—Minimum DTW distance between signatures of the symbols in the alphabet
D_{N}—Minimum distance normalized by sequence length (Dₘᵢₙ/k_{D})
Ri—Inner code rate = log₂((|A_{D}|)/k_{D}) bits nt⁻¹

We disclose the following three approaches for picking the alphabet. For all cases symbol selection is performed iteratively by evaluating simulated raw squiggle output, selecting candidate sequences, and generating and evaluating real output.

### 1. Pair-Wise Random Approach

This approach comprises computing pair-wise DTW cost between randomly generated k-mers, then picking a set where the minimum DTW cost is larger than some pre-defined threshold. Clustering algorithms, known to those skilled in the art, may also be applied to identify the best sets of symbols in terms of DTW or COW distance.

### 2. Trellis Search

Signatures for all possible 5-mers (a state of the nanopore) can be obtained from Scrappie. This would amount to 4⁵=1,024 different signatures. Using these, a trellis search can be conducted to obtain a set of sequences that generate a signature set for which the minimum pair-wise DTW distance is larger than a certain pre-set threshold (Dₘᵢₙ).

Trellis built for the search would have k_{D}−4 stages, each with 256 states, and 4 branches from each state. Search would start with a randomly generated k_{D} length DNA sequence. This would always be included in the alphabet picked. Picking a sequence for the alphabet amounts to finding a path along the trellis that creates a signature which has a DTW distance >Dₘᵢₙ with all sequences already included in the alphabet. Viterbi algorithm could be modified to find such a path.

### 3. Brute-Force Method

In this approach, DTW distance is not the metric for selecting the sequences for the alphabet A_{D}; symbol error probability itself is used. First, similar to the trellis approach, a number of random sequences of length k_{D} is generated. Signatures of all these are obtained from Scrappie. |A_{D}| sequences are randomly picked for the alphabet, and then, random squiggles are generated for each (based on the distributions obtained from Scrappie), and ‘decoded’ using the signatures. Some of the sequences will then be removed due to high symbol error probabilities. Then, another set of sequences is added to the remaining ones, and the decoding test is conducted again. Searching continues in this manner until |A_{D}| sequences are found with low symbol error rates.

### Spacer Selection and Optimisation

Spacer symbols have four main purposes:
1) to delineate the start and end of data symbols in a codeword,
2) to act as a synchronisation pattern to mark the length of known sub-sequences in an oligonucleotide strand as it translocates a nanopore at variable speed,
3) to identify template and complementary query sequences at first pass, and therefore improve decoding efficiency by informing the decoder whether decoding should be attempted against the alphabet of template or complementary data symbols, and
4) to optionally encode some additional information to increase codeword rate, distribute information across multiple different oligonucleotide fragments, provide a ‘soft’ intermediate quality control check of a query fragment, or hide information by watermarking.

Ideal properties of spacers include sequences that:
1) generate a set of current signatures sⱼ(t) that are distinctive and easily identifiable from a set of symbol signatures dᵢ(t),
2) generate mutually distinctive template and reverse complementary signatures,
3) contain a suitable GC content and
4) are of sufficient length to eliminate any interference from the upstream/previous data symbol signature di(t) so that the proceeding symbol signature dᵢ₊₁(t) is generated with predictable interference/memory from the preceding spacer sⱼ(t) and not the preceding symbol dᵢ(t).

If f bases from the quaternary alphabet A,C,T,G are simultaneously inside one nanopore at any time, and for example, f=5 say (b5, b4, b3, b2, b1), and that the output current signal A measured by the device estimates the base b3 (the middle base), there is a total number of 4⁵=1,024 possible output signals A(b)=F(b5, b4, b3, b2, b1) that will appear. The duration T of each signal may also be variable and dependent on the 5 bases, i.e., T(b)=G(b5, b4, b3, b2, b1). Given that the nanopore reading frame is f bases, and assuming f=5, and raw current measurements occur at the mid-point of the reading frame, then the number of different states q in the signature generated by a strand of DNA of length b translocating the nanopore is q=b−f+1. This implies that the total number of possible different states generated for an 8-mer DNA spacer symbol, for example, is q=8−5+1=4 states, with each of these states taking on one of 1,024 possible output signals, generating a total to 1,024⁴>1.1E12 possible signatures.

As raw data measurements occur at the mid-point of the nanopore and assuming a reading frame of 5 nucleotides for illustrative purposes, the signature produced by any DNA subsequence will be impacted by the two nucleotides immediately before and after. This means that only the middle 4-mers of an 8-mer DNA subsequence (N ˜f+1, where N is the length of a subsequence) are not affected by the memory of flanking sub-sequences. Therefore, the minimum theoretical length of the spacer/partition sequence S is k_{S}=f, but preferably k_{S}=f+1, f+2, f+3, f+4, or f+5. Optimum spacer length is a trade-off between the capacity to efficiently identify the spacers in codeword signature and information rate, bounded by f.

### Spacer Selection #1

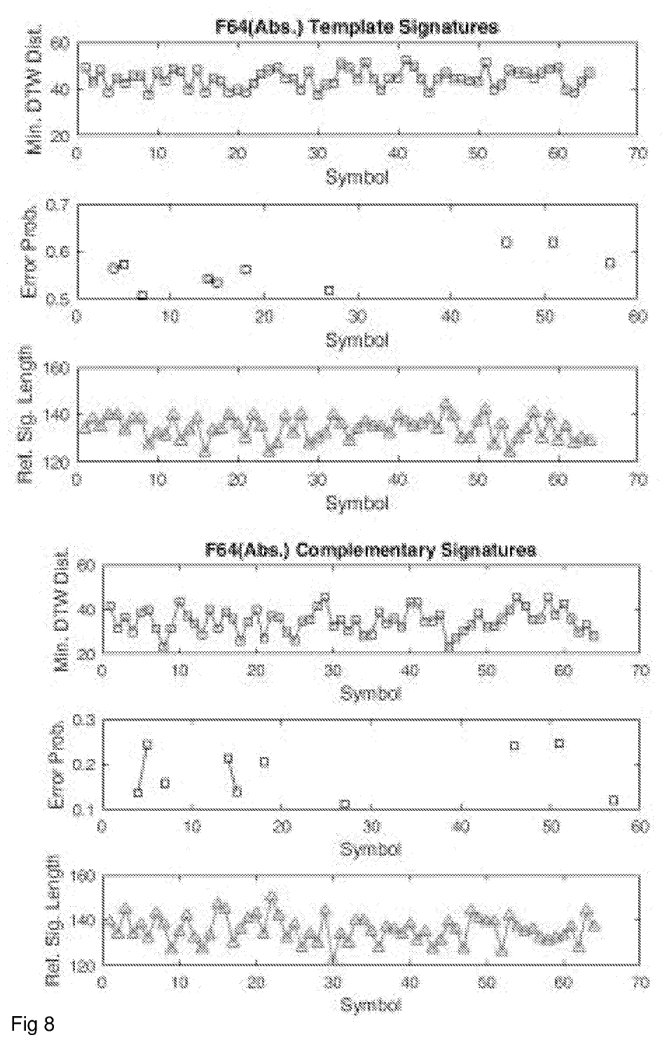

Spacer symbol selection is performed iteratively by evaluating simulated raw squiggle output, selecting candidate sequences, and generating and evaluating real output. Spacer sequence selection was first performed by simulating ‘soft’ signatures from ‘hard’ inputs using Scrappie software. Simulated signatures of the following sequences (template/reverse complementary, T/RC) were generated and evaluated against the spacer design properties outlined above. DNA tags of length n=4 were constructed with 13 of 8-mer spacer sequences listed below. Analogue signatures for a selection of the 13 spacer symbol template and reverse complementary pairs are given in FIG. 6.

S1,
AAAAAAAA/TTTTTTTT
S2,
ATATATAT/ATATATAT
S3,
AATTAATT/AATTAATT
S4,
ACACACAC/GTGTGTGT
S5,
AGAGAGAG/CTCTCTCT
S6,
AACCAACC/GGTTGGTT
S7,
AAGGAAGG/CCTTCCTT
S8,
AAATTTAA/TTAAATTT
S9,
AAACCCAA/TTGGGTTT
S10,
AAAGGGAA/TTCCCTTT
S11,
AAAATTTT/AAAATTTT
S12,
AAAACCCC/GGGGTTTT
S13,
AAAAGGGG/CCCCTTTT

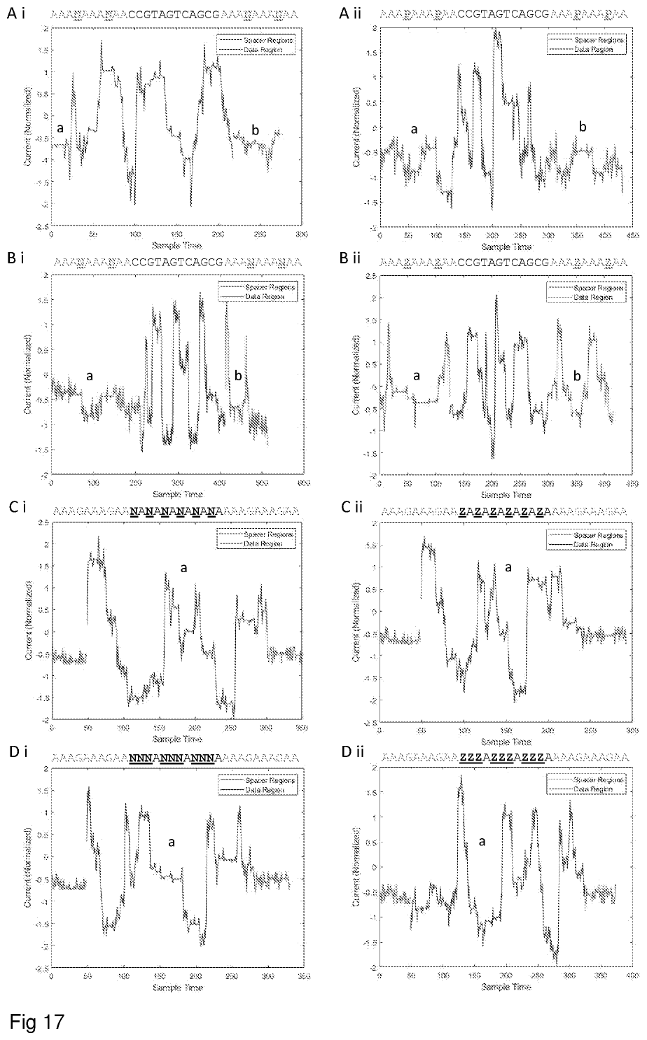

Mean signatures of ID tags were simulated using Scrappie software and evaluated as spacers. These simulations are provided in FIG. 6. Spacers that performed well in theoretical simulations were manufactured into tags, sequenced, and the real raw data further evaluated. Within certain parameters, all of the tested sequences may be used as spacers, although some sequences performed significantly better than others. For example, poly-A spacers generate a relatively ‘flat’ and distinctive signature which is easily detectable. This property lowers the latency of spacer detection which improves the throughput of the system. A ‘flat’ signature may be desirable since random changes in translocation duration, or the ‘time warp’, will not affect the detection of such a signature. However, mean amplitude of a poly-A sequence is very similar to the mean amplitude of its reverse complementary, poly-T sequence, thus making template and reverse complementary strand classification from the spacers alone difficult. Additionally, the high A and T content somewhat restricts symbol selection. Therefore, poly-A sequences may not be optimal. High amplitude ‘spikey’ spacers may also be desirable for detection, which may be constructed from TGA repeats. Furthermore, desirable spacer properties may also be achieved by incorporating one or more unnatural AEGIS bases of the set {Z, P, B, S} as shown in FIG. 17.

Spacers and spacer-symbols may be of size k_{S}=5-16 nt, preferably 6-14 nt, preferably 6-12 nt, preferably 8-12 nt. In general spacers are of size f≤k_{S}≤2f, where f is the number of bases in an oligonucleotide fragment that translocate a nanopore at any one time. Spacers may be any sequence, but preferably:
A homopolymer comprised of one of the set {A} or {T}
An alternating copolymer comprised of two species of alternating monomeric nucleotides {A, T} or {A, C} or {A, G}
An alternating copolymer comprised of two species of alternating dimeric nucleotides {AA, TT} or {AA, CC} or {AA, GG}
An alternating copolymer comprised of three species of alternating trimeric nucleotides {AAA, TTT} or {AAA, CCC} or {AAA, GGG}
An alternating copolymer comprised of four species of alternating tetrameric nucleotides {AAAA, TTTT} or {AAAA, CCCC} or {AAAA, GGGG}
A sequence containing one or more repeats of {AAAG} and/or {AAG}
A sequence containing one or more repeats of {TGA}
A sequence containing one or more AEGIS base of the set {Z, P, S, B}

### Spacer Selection #2

A more structured way of searching is choosing spacer sequences through brute force. The brute force method of searching involves generating an exhaustive or near-exhaustive set of possible spacer sequences of length k_{S}, and picking symbols that generate a signature/s of a desired shape/s. After generating a set of random ‘hard’ sequences scrappie software was used to generate the corresponding average ‘soft’ current signatures. These signatures were then compared with the desired pattern/s, and close matches were picked as spacers. Again, brute force spacer symbol selection is performed iteratively by evaluating simulated raw squiggle output, selecting candidate sequences, and generating and evaluating real output.

Spacers and spacer-symbols may be of size k_{S}=5-16 nt, preferably 6-14 nt, preferably 6-12 nt, preferably 8-12 nt. Spacers are of size f≤k_{S}≤2f, where f is the number of bases in an oligonucleotide fragment that translocate a nanopore at any one time.

### Multiple Spacers to Increase Codeword Rate

Here we disclose a method for increasing codeword rate r by using two alphabets, A_{D} and A_{S}, for an ID tag. The tag is constructed from alternating symbols from A_{D} and A_{S}, with each tag containing n symbols from A_{D} and n+1 symbols from A_{S}, as shown in FIG. 4. The size of the data symbol alphabet is typically larger than the spacer symbol alphabet, or |A_{D}|>|A_{S}|. The spacer alphabet A_{S} is typically smaller because it must meet both symbol and spacer design constraints. In most cases |A_{S}|≤16 or preferably ≤8 and |A_{D}|≥16. For example, consider:
   |A_{D}|=2⁸=256 symbols, of length k_{D}=12 nt and rate r=0.67 bits nt⁻¹
   |A_{S}|=2²=16 spacer symbols, of length k_{S}=8 nt and rate r=0.5 bits nt⁻¹

For an alternating tag of length n=4 that is comprised of 4 symbols from A_{D} and 5 symbols from A_{S}, i.e. Sⱼ₁Dᵢ₁Sⱼ₂Dᵢ₂Sⱼ₃Dᵢ₃Sⱼ₄Dᵢ₄Sⱼ₅ the total number of bits encoded is 52 over an encoding region of 88 nucleotides, which equates to a rate of 0.593 bits nt⁻¹. If spacers are not used to encode information, the equivalent codeword would contain 32 bits over an encoding region of 88 nucleotides, which equates to a rate of 0.366 bits nt⁻¹.

The alphabets A_{D} and A_{S} may be of any size, and comprised of symbols and spacer symbols of size k_{D/S}=5-16 nt, preferably 6-14 nt preferably 6-12 nt, preferably 8-12 nt. Spacers are of size f≤k_{S}≤2f, where f is the number of bases in an oligonucleotide fragment that translocate a nanopore at any one time.

### Multiple Spacer-Symbols to Distribute Information Across Multiple DNA Fragments

Multiple spacers may also be used to encode information across multiple oligonucleotide strands in circumstances where it is desirable to use short oligonucleotide fragments (i.e <200 nt), and there is a need to encode more information than can fit in a single fragment alone. In many cases short fragments are desirable because they are less likely to degrade, are less expensive to manufacture (both in terms of per nucleotide length and per mol) and are subject to lower synthesis error rate.

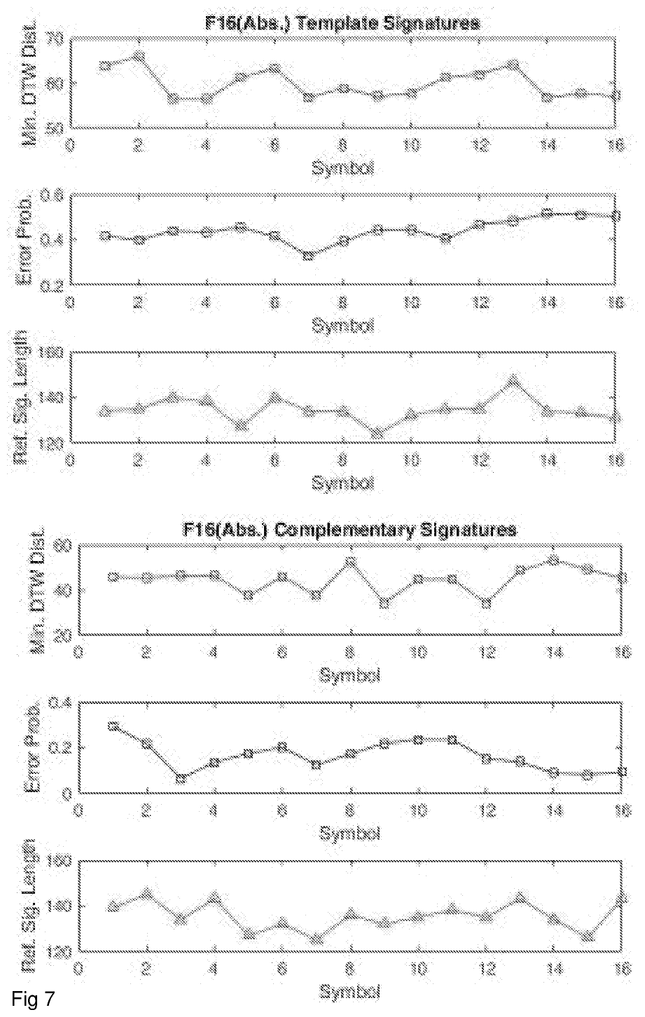

Here we disclose a method to use spacers to encode an index to address individual strands to a location in a multi-strand ID tag or ‘datablock’. Refer also to FIG. 5 which illustrates how spacers may be used to distribute information across multiple DNA strands.

Consider the following example:
   |A_{D}|=2⁸⁼²⁵⁶ symbols, of length k_{D}=12 nt and rate r=0.67 bits nt⁻¹
   |A_{S}|=2¹=2 spacer symbols of length k_{S}=8 nt and r=0.125 bits nt⁻¹

For an alternating ID tag of length n=4 that is comprised of 4 symbols from A_{D} and 5 symbols from A_{S}, i.e. Sⱼ₁Dᵢ₁Sⱼ₂Dᵢ₂Sⱼ₃Dᵢ₃Sⱼ₄Dᵢ₄Sⱼ₅ there 2564=4.3 billion possible A_{D} tags and 2⁵=32 A_{S} tags. In this embodiment, the A_{S} tags are used as an index to assemble the A_{D} tags into a ‘datablock’ or multistrand ID tag. This approach permits an essentially unlimited number of 32^{256\hat{ }4} unique data blocks, although for practical applications each data block is not required to contain the full set of A_{S} tags. If only four A_{S} tags are used, for example, this would permit a multistrand ID tag space of 4^{256\hat{ }4}.

The alphabets A_{D} and A_{S} may be of any size, and comprised of symbols and spacer symbols of size k_{D}/S=5-16 nt, preferably 6-14 nt preferably 6-12 nt, preferably 8-12 nt. Spacers are of size f≤k_{S}≤2f, where f is the number of bases in an oligonucleotide fragment that translocate a nanopore at any one time.

### Multiple Spacers to Hide Information by Watermarking

Watermarking is the process of hiding information in a carrier signal to improve security. Here we disclose a methodology for DNA watermarking, where one or more oligonucleotide single strand ID tags, or one or more oligonucleotide ‘blocks’ or multistrand ID tags, or a combination of one or more oligonucleotide single strand ID tags and oligonucleotide blocks or multistrand ID tags, is hidden in a larger pool of oligonucleotide fragments. Consider oligonucleotide ID tags comprised of alternating symbols from a set of data symbols (alphabet A_{D}) and a set spacer symbols (alphabet A_{S}). Water marking is achieved by using the alphabet A_{S} to encode information that identifies the correct tag/s in a larger set of tags. For example:
   |A_{D}|=2⁸=256 symbols, of length k_{D}=12 nt and rate r=0.67 bits nt⁻¹
   |A_{S}|=2⁶=64 spacer symbols, of length k_{S}=8 nt and rate r=0.75 bits nt⁻¹

For an alternating ID tag of length n=4 that is comprised of 4 symbols from A_{D} and 5 symbols from A_{S}, i.e. Sⱼ₁Dᵢ₁Sⱼ₂Dᵢ₂Sⱼ₃Dᵢ₃Sⱼ₄Dᵢ₄Sⱼ₅ there is a total of 64⁵=1.074 billion possible configurations from the set A_{S}. One or more configuration from the set A_{S} may be used to identify the correct ID tag/information from a larger pool of ‘plausible’ tags. Plausible tags include any oligonucleotide strand encoded from the same alphabets and with the same parameterisation/form as correct tags, e.g. Sⱼ₁Dᵢ₁Sⱼ₂Dᵢ₂Sⱼ₃Dᵢ₃Sⱼ₄Dᵢ₄Sⱼ₅. Pools of >100,000 plausible oligonucleotide tags may be synthesised by commercial manufacturers such as IDT and Twist BioSciences. These pools may be added to the ‘correct’ tag/s at the same or similar molar concentration to achieve watermarking.

The alphabets A_{D} and A_{S} may be of any size, and comprised of symbols and spacer symbols of size k_{D/S}=5-16 nt, preferably 6-14 nt preferably 6-12 nt, preferably 8-12 nt. Spacers are of size f≤k_{S}≤2f, where f is the number of bases in an oligonucleotide fragment that translocate a nanopore at any one time.

In some embodiments, it may be advantageous to perform tag decoding locally and watermark decoding locally, whist in other embodiments it may be advantageous to perform tag decoding locally watermark decoding remotely, and in yet still other embodiments it may be advantageous to perform tag decoding remotely and watermark decoding remotely.

### Outer Codes to Increase Error Detection and Correction

Outer codes were also tested to improve error detection and correction capability. In some embodiments, the codeword is constructed with an inner code of ‘soft’ analogue symbols in combination with a ‘hard’ outer code. In these embodiments the inner ‘soft’ symbols may be mers of length 5-16 nt and selected using minimum mutual absolute or Euclidean distance in DTW as a metric. The outer ‘hard’ code may include linear block codes, for example: cyclic codes (e.g. Hamming codes), repetition codes, parity codes, polynomial codes, Reed-Solomon codes, algebraic geometric codes, or Reed-Muller codes. The outer ‘hard’ code may also include convolutional codes and product (block turbo) codes.

In one example, codewords were constructed from k_{D}=12-mer data symbols selected using a minimum mutual absolute distance in DTW threshold of 44.5 over F64. Data symbols from A_{D} were arranged into an alternating Hamming [n, k] codeword where n=7 and k=4, and where each D was flanked by an S. This gives the outer code C_{D} an error detection capacity of two symbols and error correction capacity of one symbol.

In other embodiments, the ‘soft’ analogue inner symbols are assembled into a codeword using a soft outer code. This soft outer code may include codes optimised for soft decoding such as a convolutional code, an LDPC code, or a turbo code.

In all embodiments, the outer code may be applied to the symbols of A_{D} or the symbols of A_{S}, or both the symbols of A_{D} and A_{S}, in an alternating codeword comprised of alternating symbols from A_{D} and A_{S}.

A similar scheme to using multiple fragments for a single message is one where we use a long outer code, such as a good NB-LDPC code. In this case, we first construct a codeword from the alphabet A_{D} of length K(|A_{S}|−1), where K is the number of codeword ‘segments’. Then this codeword is divided into K segments, each of length |A_{S}|−1. The location of each segment in the long codeword is encoded using the spacer (or A_{S}) alphabet. Since long codewords have better performance than shorter ones, a scheme like this can be expected to improve performance. But, once more, at least one read of each segment of data is used for decoding the outer code, which might impact the efficiency of the system. Note that the example with codewords of length K(|A2|−1) was just an example case, in general the outer code would be of length KL, with L<=A_{S}|^{(K+1)}.

### A Methodology to Increase Information Rate and Improve Alphabet Design

Here we disclose a method to include unnatural ‘Hachimoji’ or ‘AEGIS’ nucleotides into synthetic oligonucleotide tags to increase the information rate and give better data and spacer alphabet design flexibility. AEGIS nucleotides include the pyrimidine bases Z and S and the purine bases P and B, which form the complementary hydrogen bonding pairs Z:P and S:B. AEGIS bases may be used to expand the number of nucleotides used to encode information in an oligonucleotide from four to eight, and thereby increase the theoretical maximum information density from 2 bits nt-1 to 3 bits nt-1. Data presented in FIG. 17 show the surprising result that AEGIS bases incorporated into spacer and data symbols are detectable using nanopore sequencing and the methodologies disclosed previously.

For the purpose of generating the figures, first some sequences containing AEGIS bases were designed, and manufactured. Then, those were sequenced using a nanopore device, first without the unnatural AEGIS bases present for the PCR amplification, and then with dNTPs only. The raw signals resulting from the sequencing runs were then clustered based on pair-wise DTW distance, and a consensus signal was generated for each primary cluster using DTW Barycenter Averaging (DBA). The regions of the consensus signals that are generated by the sequences containing the AEGIS bases were found by first locating the regions for the adjacent sub-sequences that do not contain the AEGIS bases, once more using DTW distances.

The inclusion of AEGIS bases may be used to generate a larger range of different raw current signatures, and thereby permit greater flexibility in data and spacer alphabet design. For example, by using symbol selection methodologies disclosed previously, data alphabet symbols A_{D} and spacer alphabet symbols A_{S} may be generated at larger mutual DTW and/or COW distance which may increase decoding efficiency and reliability. Additionally, AEGIS bases may be used to design larger data |A_{D}| and spacer alphabets |A_{S}| for a given minimum mutual DTW and/or COW distance compared to the same size alphabets constructed from conventional nucleotides alone. This surprising result permits the design of nanopore encoding systems with greater flexibility, improved information density, and improved decoding and sequence identification reliability.

### Decoding Algorithm

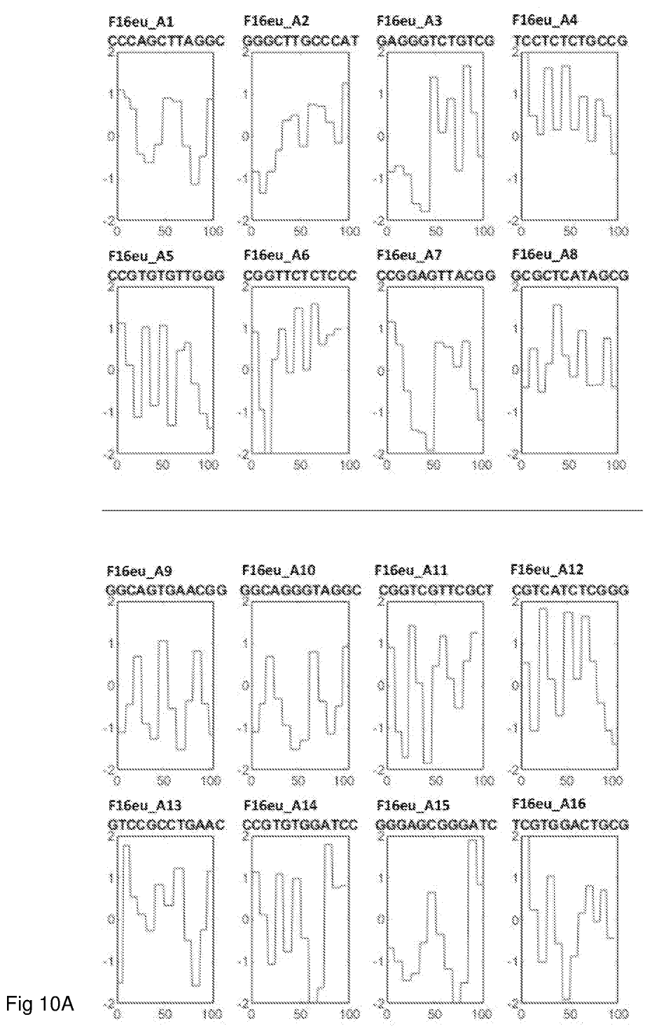

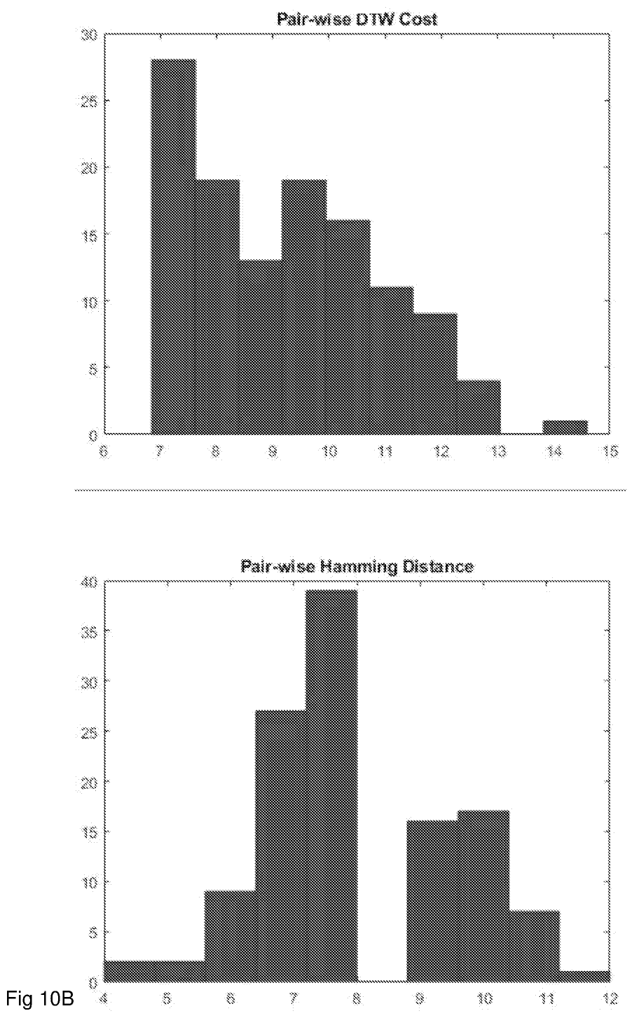

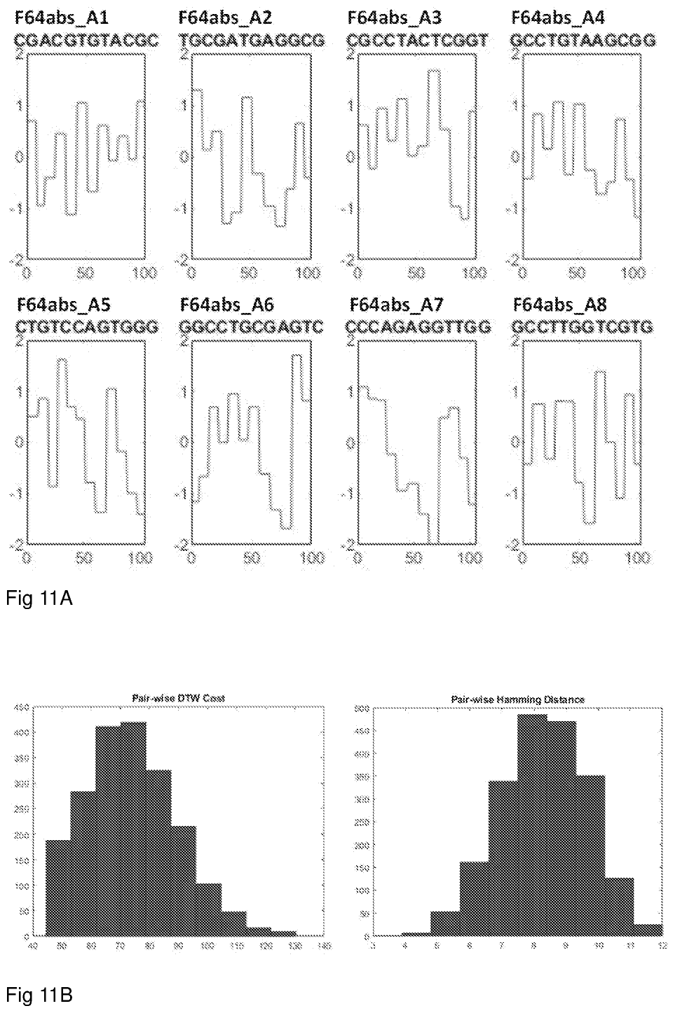

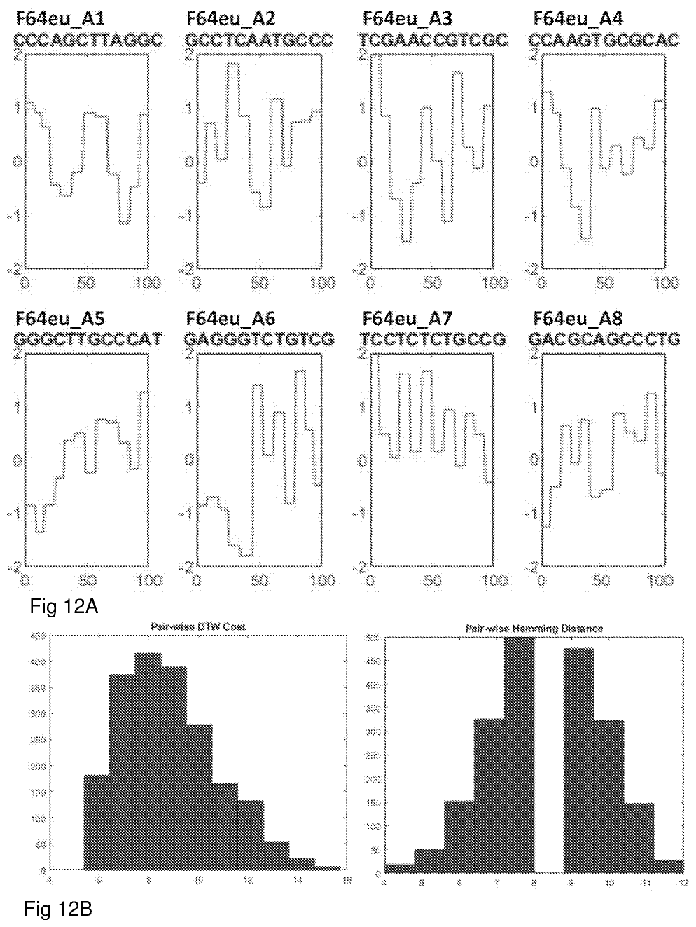

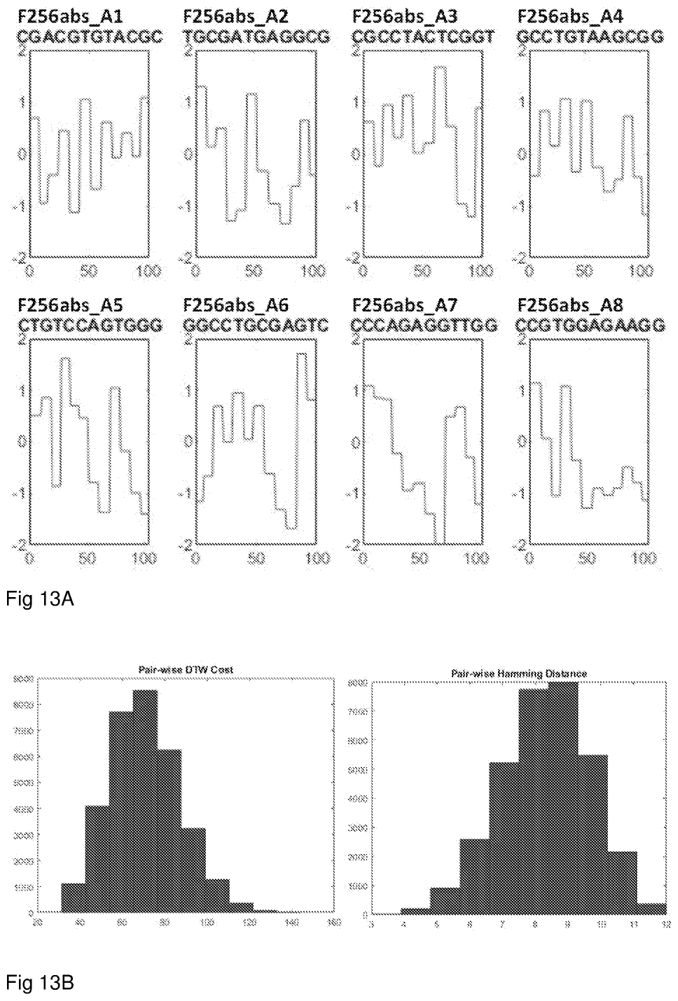

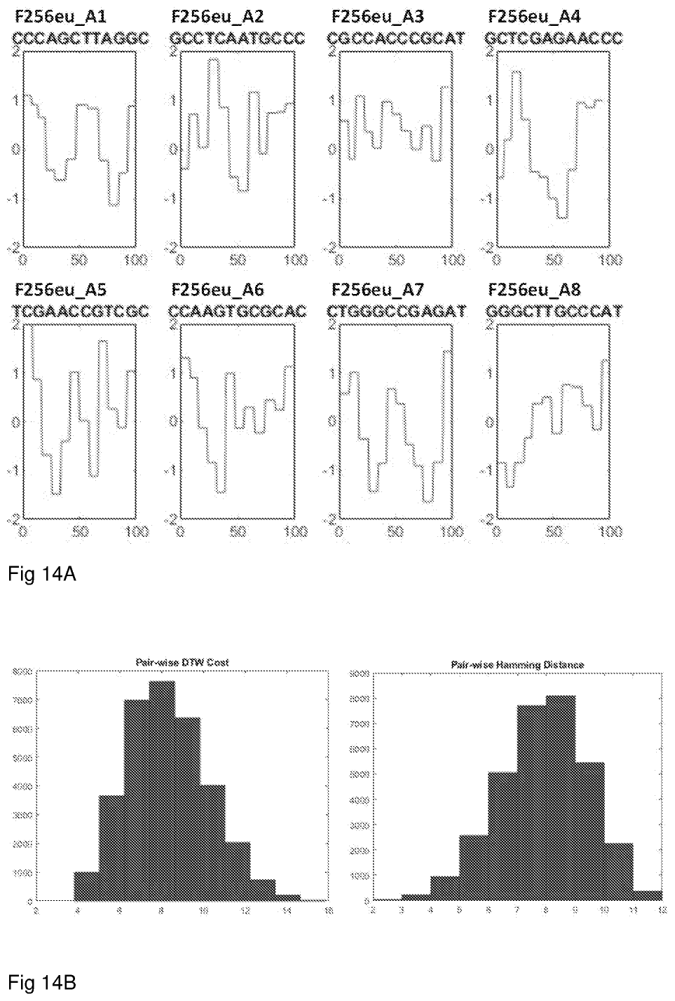

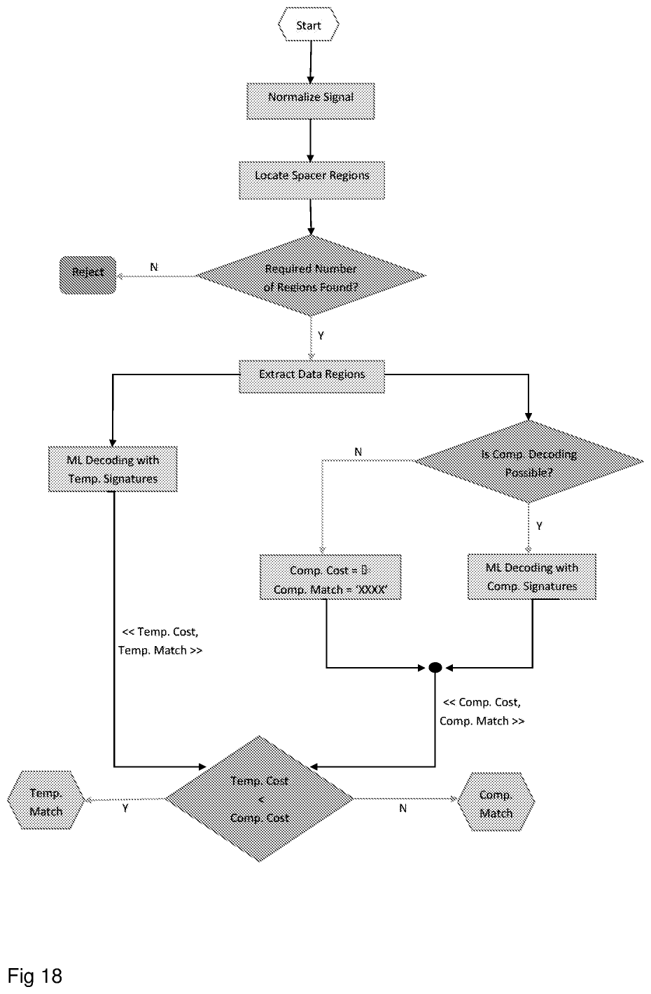

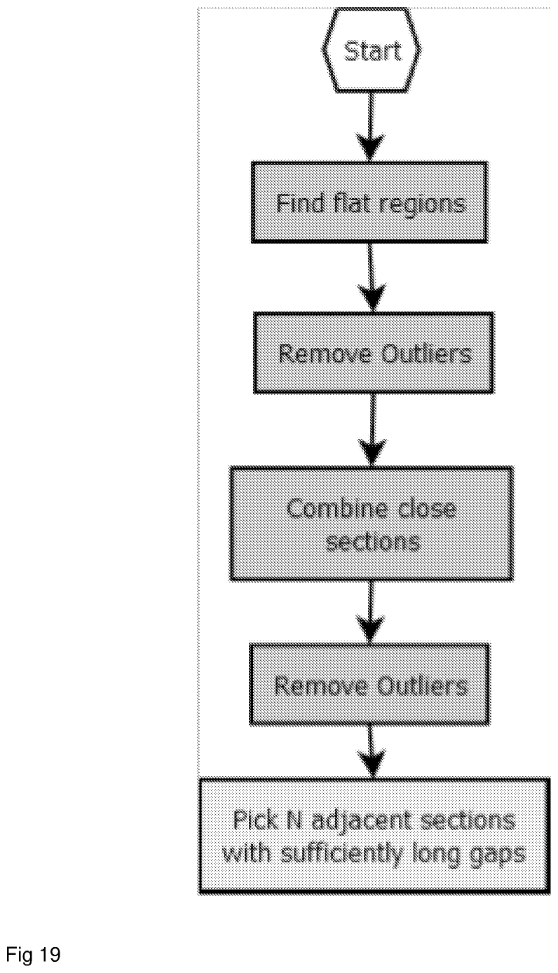

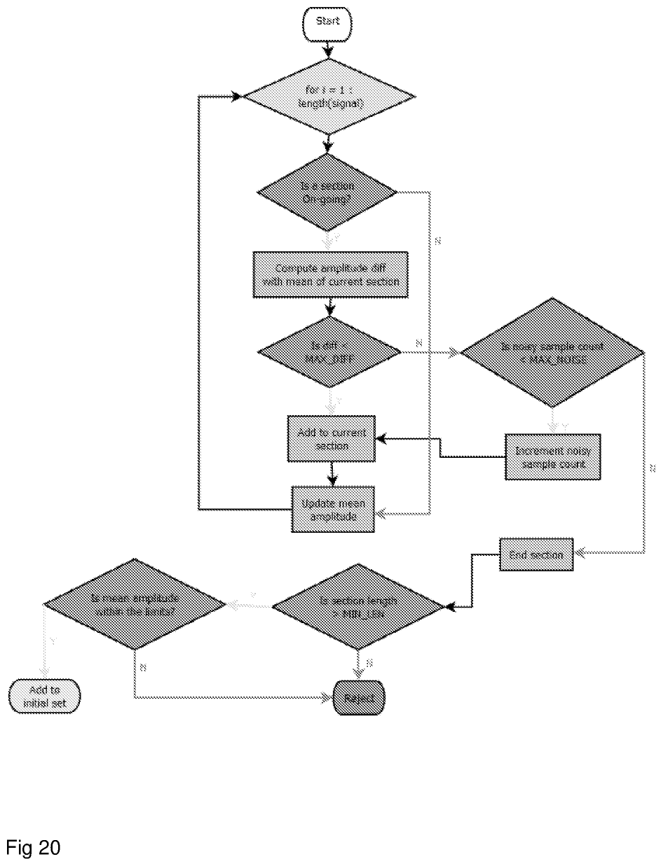

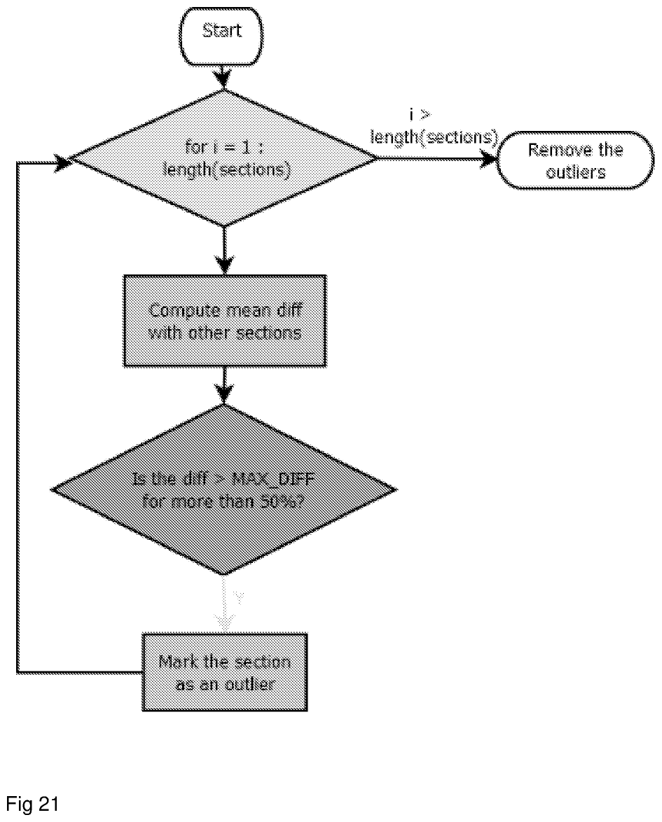

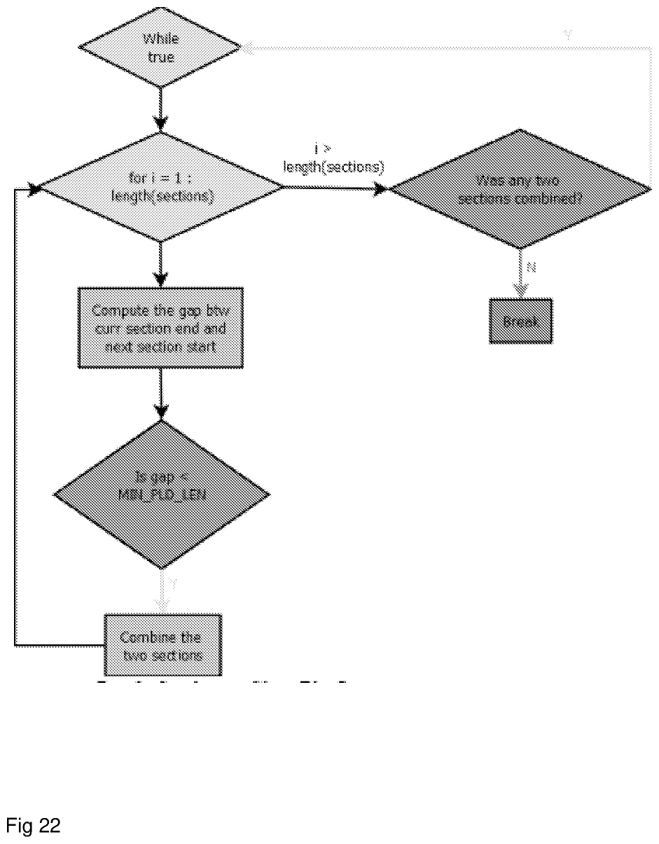

FIG. 18 gives an overview of how decoding is carried out with nanopore signals. Note that maximum likelihood (ML) decoding is replaced with a suitable decoding algorithm when longer codes or larger alphabets or outer codes are used. Alphabets given in FIG. 9-14, SeqID NO: 1-672, were generated using either Euclidean distance, or absolute distance, as the distance metric in DTW. Both types of alphabets seem to perform reasonably well, with absolute distance alphabets outperforming the other (marginally) in 2 of the 3 cases.

In cases where outer codes are not used, the best option may be to use a maximum likelihood (ML) or a ML-based approach using any suitable distance metric, such as DTW. The most suitable distance metrics may be those that are closest to actual probabilities.

In cases where outer codes are used, decoding would depend on which code, and which codeword length, is used. For short codes over a small alphabet, such as a (n, k), where n is the codeword length and k is the number of data symbols, for e.g. (7, 4) over F16, the DTW cost vectors obtained from decoding the inner code can be used for ML decoding of the outer code. For longer codes, or ones using larger alphabets, ML is not practical, in which case a more suitable decoder is used; e.g.: BP for LDPC, Chase-Pyndiah decoding for product codes, etc. If the outer code is hard decoded, then it would work with the ML estimates for each symbol obtained from inner decoding. Once more, the specific decoding algorithm would depend on the code; eg: Berlekamp algorithm for RS codes, iterative hard decoding with product codes, etc. A number of codes would perform reasonably well with BP decoding (hard or soft), but suitable parity-check matrices are first computed for them. Chase decoding is a good option for soft decoding any algebraic code.

Machine learning is an alternative approach that may be used for decoding. It may be used for data decoding, after the spacer decoding step in FIG. 18 or may be used for decoding both spacer and data symbols. In both cases, the neural network used for decoding should be trained on sequences constructed from the identified alphabets with large amounts of ‘noisy’ data for which the underlying sequences/symbols are known. With the network trained sufficiently well, the raw signals generated when reading a DNA strand could be directly fed to it, and it would output the most likely sequence/symbol.

### Example 1—Absolute Distance in DTW as a Metric for Symbol Selection

To demonstrate our encoding approach using absolute distance in DTW to select A_{D}, 500 symbols of each length k_{D}=8, 10, 12, 14 and 16 were randomly generated within the following constraints:
Each data sequence of a symbol cannot start with the same nucleotide as the end of the spacer sequence, or end with the same nucleotide as the start of the spacer sequence.
The maximum GC content in a symbol is ≤70%
The maximum G or C homopolymer region in a symbol is ≤3

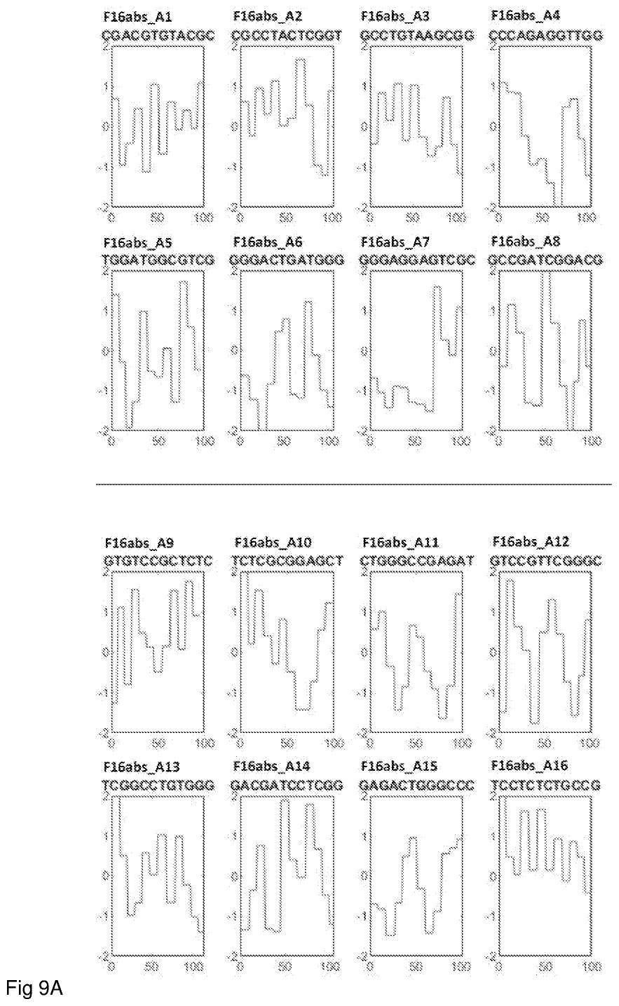

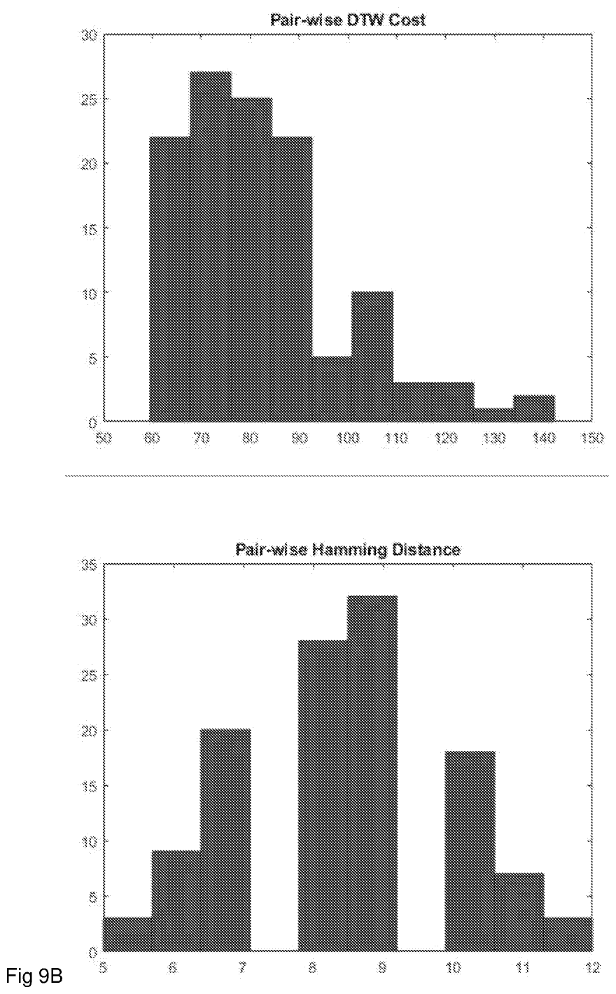

The analogue current signatures of each k_{D} length set of 500 symbols were then simulated using Scrappie software. Alphabets of size |A_{D}|=16, 64 and 256 were then selected from the 500 simulated signatures using a minimum absolute distance in dynamic time warping (DTW) threshold of 59.5, 44.5 and 31.5, respectively (See Table 1). Error probabilities for template and complementary current signature for symbols in the F16 and F64 alphabets are given in FIG. 7 and FIG. 8, respectively. The sets of data symbol sequences for these F16, F64 and F256 alphabets were selected using minimum absolute distance in DTW are given in Tables 11-16 and corresponding simulated current signatures di(t) are given in FIG. 9-FIG. 14.

ID tags given below (ID_F16abs_001-012, ID_F64abs_001-004, and ID_F256abs_001-004) were synthesised by Macrogen and sequenced using the Oxford Nanopore MinION device and SQK-LSK109 protocol with R9.4.1 flowcells. The resulting raw analogue data in .fast5 file format was inputted into the decoder. Results for alphabets of size |A_{D}|=16, 64, and 256 are given in Table 4, Table 5 and Table 6, respectively.

Results show that data symbol alphabets constructed using absolute distance in DTW outperformed those constructed using Euclidean distance in DTW, for |A_{D}|<64.

TABLE 4
Decoding results for Sⱼ₁Dᵢ₁Sⱼ₁Dᵢ₂Sⱼ₁Dᵢ₃Sⱼ₁Dᵢ₄Sⱼ₁
ID tags constructed from an A_{D} alphabet of symbols selected at a minimum
mutual absolute distance of 59.9 where |A_{D}| = 16.

| ID Tag | Total Reads | Not Usable | Errors | Matches Temp. | Comp. | Total |
|---|---|---|---|---|---|---|
| ID_F16abs_001 | 4731 | 1362 | 1761 | 842 | 766 | 1608 |
| | | (28.8%) | (37.2%) | (17.8%) | (16.2%) | (34%) |
| ID_F16abs_002 | 6567 | 1651 | 2067 | 1473 | 1376 | 2849 |
| | | (25.1%) | (31.5%) | (22.4%) | (21%) | (43.4%) |
| ID_F16abs_003 | 3837 | 1058 | 1311 | 849 | 619 | 1468 |
| | | (27.6%) | (34.2%) | (22.1%) | (16.1%) | (38.3%) |
| ID_F16abs_004 | 5337 | 1516 | 1630 | 1023 | 1168 | 2191 |
| | | (28.4%) | (30.5%) | (19.2%) | (21.9%) | (41.1%) |
| ID_F16abs_005 | 8605 | 2438 | 3257 | 1737 | 1173 | 2910 |
| | | (28.3%) | (37.9%) | (20.2%) | (13.6%) | (33.8%) |
| ID_F16abs_006 | 3716 | 1092 | 1135 | 748 | 741 | 1488 |
| | | (29.4%) | (30.5%) | (20.1%) | (19.9%) | (40%) |
| Total | 32793 | 9117 | 11161 | 6672 | 5843 | 12515 |
| | | (27.8%) | (34%) | (20.3%) | (17.8%) | (38.2%) |

TABLE 5
Decoding results for Sⱼ₁Dᵢ₁Sⱼ₁Dᵢ₂Sⱼ₁Dᵢ₃Sⱼ₁Dᵢ₄Sⱼ₁
ID tags constructed from an A_{D} alphabet of symbols selected at a minimum
mutual absolute distance of 44.5 where |A_{D}| = 64.

| ID Tag | Total Reads | Not Usable | Errors | Matches Temp. | Comp. | Total |
|---|---|---|---|---|---|---|
| ID_F64abs_001 | 5909 | 1728 | 2192 | 1045 | 944 | 1989 |
| | | (29.2%) | (37.1%) | (17.7%) | (16%) | (33.7%) |
| ID_F64abs_002 | 5242 | 1479 | 1991 | 962 | 810 | 1772 |
| | | (28.2%) | (38%) | (18.4%) | (15.5%) | (33.8%) |
| ID_F64abs_003 | 4988 | 1554 | 2181 | 619 | 634 | 1253 |
| | | (31.2%) | (43.7%) | (12.4%) | (12.7%) | (25.1%) |
| ID_F64abs_004 | 5908 | 2571 | 1991 | 782 | 564 | 1346 |
| | | (43.5%) | (33.7%) | (13.2%) | (9.5%) | (22.8%) |
| Total | 22047 | 7332 | 8355 | 3408 | 2952 | 6360 |
| | | (33.3%) | (37.9%) | (15.5%) | (13.4%) | (28.8%) |

TABLE 6
Decoding results for Sⱼ₁Dᵢ₁Sⱼ₁Dᵢ₂Sⱼ₁Dᵢ₃Sⱼ₁Dᵢ₄Sⱼ₁
ID tags constructed from an A_{D} alphabet of symbols selected at a minimum
mutual absolute distance of 31.5 where |A_{D}| = 256.

| ID Tag | Total Reads | Not Usable | Errors | Matches Temp. | Comp. | Total |
|---|---|---|---|---|---|---|
| ID_F256abs_001 | 5367 | 1855 | 2421 | 558 | 533 | 1091 |
| | | (34.6%) | (45.1%) | (10.4%) | (9.9%) | (20.3%) |
| ID_F256abs_002 | 4425 | 1476 | 2020 | 565 | 364 | 929 |
| | | (33.4%) | (45.6%) | (12.8%) | (8.2%) | (21%) |
| ID_F256abs_003 | 4509 | 1286 | 2501 | 369 | 353 | 722 |
| | | (28.5%) | (55.5%) | (8.2%) | (7.8%) | (16%) |
| ID_F256abs_004 | 7204 | 2450 | 3072 | 989 | 693 | 1682 |
| | | (34%) | (42.6%) | (13.7%) | (9.6%) | (23.3%) |
| Total | 21505 | 7067 | 10014 | 2481 | 1943 | 4424 |
| | | (32.9%) | (46.6%) | (11.5%) | (9%) | (20.6%) |

F16, Absolute Distance, Spacer 1
ID_F16abs_001: S1/SEQ ID NO: 1/S1/SEQ ID NO: 2/S1/SEQ ID NO: 3/S1/SEQ ID NO: 4/S1
ID_F16abs_002: S1/SEQ ID NO: 5/S1/SEQ ID NO: 6/S1/SEQ ID NO: 7/S1/SEQ ID NO: 8/S1
ID_F16abs_003: S1/SEQ ID NO: 9/S1/SEQ ID NO: 10/S1/SEQ ID NO: 11/S1/SEQ ID NO: 12/S1
ID_F16abs_004: S1/SEQ ID NO: 13/S1/SEQ ID NO: 14/S1/SEQ ID NO: 15/S1/SEQ ID NO: 17/S1
ID_F16abs_005: S1/SEQ ID NO: 1/S1/SEQ ID NO: 5/S1/SEQ ID NO: 9/S1/SEQ ID NO: 13/Sᵢ
ID_F16abs_006: S1/SEQ ID NO: 4/S1/SEQ ID NO: 18/S1/SEQ ID NO: 12/S1/SEQ ID NO: 16/S1

F64, Absolute Distance, Spacer 1
ID_F64abs_001: S1/SEQ ID NO: 34/S1/SEQ ID NO: 35/S1/SEQ ID NO: 84/S1/SEQ ID NO: 80/S1
ID_F64abs_002: S1/SEQ ID NO: 59/S1/SEQ ID NO: 35/S1/SEQ ID NO: 84/S1/SEQ ID NO: 80/S1
ID_F64abs_003: S1/SEQ ID NO: 56/S1/SEQ ID NO: 48/S1/SEQ ID NO: 81/S1/SEQ ID NO: 94/S1
ID_F64abs_004: S1/SEQ ID NO: 35/S1/SEQ ID NO: 84/S1/SEQ ID NO: 80/S1/SEQ ID NO: 92/S1

F256, Absolute Distance, Spacer 1
ID_F256abs_001: S1/SEQ ID NO: 184/S1/SEQ ID NO: 242/S1/SEQ ID NO: 307/S1/SEQ ID NO: 261/S1
ID_F256abs_002: S1/SEQ ID NO: 364/S1/SEQ ID NO: 242/S1/SEQ ID NO: 307/S1/SEQ ID NO: 261/S1
ID_F256abs_003: S1/SEQ ID NO: 270/S1/SEQ ID NO: 173/S1/SEQ ID NO: 209/S1/SEQ ID NO: 285/S1
ID_F256abs_004: S1/SEQ ID NO: 242/S1/SEQ ID NO: 174/S1/SEQ ID NO: 261/S1/SEQ ID NO: 328/S1

### Example 2—Euclidean Distance in DTW as a Metric for Symbol Selection

To demonstrate our encoding approach using Euclidean distance in DTW to select A_{D}, 500 symbols of each length k_{D}=8, 10, 12, 14 and 16 were randomly generated within the following constraints:
Each data sequence of a symbol cannot start with the same nucleotide as the end of the spacer sequence, or end with the same nucleotide as the start of the spacer sequence.
The maximum GC content in a symbol is ≤70%
The maximum G or C homopolymer region in a symbol is ≤3

The analogue current signatures of each k_{D} length set of 500 symbols was then simulated using Scrappie software. Alphabets of size |A_{D}|=16, 64 and 256 were then selected from the 500 simulated signatures using a minimum Euclidean distance in dynamic time warping (DTW) threshold of 6.8, 5.375 and 3.825, respectively (See Table 1). The sets of data symbol sequences for these F16, F64 and F256 alphabets selected using minimum Euclidean distance in DTW are given in Tables 11-16 and corresponding simulated current signatures di(t) are given in FIG. 9-FIG. 14.

ID tags listed below (ID_F16eu_001-012, ID_F64eu_001-004, and ID_F256eu_001-004) were synthesised by Macrogen and sequenced using the Oxford Nanopore SQK-LSK109 protocol and R9.4.1 flowcells. The resulting raw analogue data in .fast5 file format was inputted into the decoder. Results for alphabets of size |A_{D}|=16, 64, and 256 are given in Table 7Error! Reference source not found, Table 8, and Table 9, respectively.

Results show that data symbol alphabets constructed using Euclidean distance in DTW outperformed those constructed using absolute distance in DTW, for |A_{D}|>64.

TABLE 7
Decoding results for Sⱼ₁Dᵢ₁Sⱼ₁Dᵢ₂Sⱼ₁Dᵢ₃Sⱼ₁Dᵢ₄Sⱼ₁
ID tags constructed from an A_{D} alphabet of symbols selected at a minimum
mutual Euclidean distance of 6.8 where |A_{D}| = 16.

| ID Tag | Total Reads | Not Usable | Errors | Matches Temp. | Comp. | Total |
|---|---|---|---|---|---|---|
| ID_F16eu_001 | 5131 | 1702 | 1712 | 692 | 1025 | 1717 |
| | | (33.2%) | (33.4%) | (13.5%) | (20%) | (33.5%) |
| ID_F16eu_002 | 8312 | 2739 | 2984 | 1123 | 1466 | 2589 |
| | | (33%) | (35.9%) | (13.5%) | (17.6%) | (31.1%) |
| ID_F16eu_003 | 4000 | 1207 | 1487 | 652 | 654 | 1306 |
| | | (30.1%) | (37.2%) | (16.3%) | (16.4%) | (32.7%) |
| ID_F16eu_004 | 11055 | 2966 | 3847 | 2335 | 1907 | 4242 |
| | | (26.8%) | (34.8%) | (21.1%) | (17.3%) | (38.4%) |
| ID_F16eu_005 | 5203 | 1323 | 2149 | 904 | 827 | 1731 |
| | | (25.4%) | (41.3%) | (17.4%) | (15.9%) | (33.3%) |
| ID_F16eu_006 | 11479 | 4085 | 3897 | 1515 | 1982 | 3497 |
| | | (35.6%) | (33.9%) | (13.2%) | (17.3%) | (30.5%) |
| Euc. Dist | 45180 | 14022 | 16076 | 7221 | 7861 | 15082 |
| | | (31%) | (35.6%) | (16%) | (17.4%) | (33.4%) |

TABLE 8
Decoding results for Sⱼ₁Dᵢ₁Sⱼ₁Dᵢ₂Sⱼ₁Dᵢ₃Sⱼ₁Dᵢ₄Sⱼ₁
ID tags constructed from an A_{D} alphabet of symbols selected at a minimum
mutual Euclidean distance of 5.375 where |A_{D}| = 64.

| ID Tag | Total Reads | Not Usable | Errors | Matches Temp. | Comp. | Total |
|---|---|---|---|---|---|---|
| ID_F64eu_001 | 4664 | 1483 | 1988 | 737 | 456 | 1193 |
| | | (31.8%) | (42.6%) | (15.8%) | (9.8%) | (25.6%) |
| ID_F64eu_001 | 6842 | 2396 | 2754 | 907 | 785 | 1692 |
| | | (35%) | (40.2%) | (13.3%) | (11.5%) | (24.7%) |
| ID_F64eu_001 | 6606 | 1980 | 2841 | 887 | 898 | 1785 |
| | | (30%) | (43%) | (13.4%) | (13.6%) | (27%) |
| ID_F64eu_001 | 2444 | 884 | 991 | 298 | 271 | 569 |
| | | (36.2%) | (40.5%) | (12.2%) | (11.1%) | (23.3%) |
| Euc. Dist | 20556 | 6743 | 8574 | 2829 | 2410 | 5239 |
| | | (32.8%) | (41.7%) | (13.8%) | (11.7%) | (25.5%) |

TABLE 9
Decoding results for Sⱼ₁Dᵢ₁Sⱼ₁Dᵢ₂Sⱼ₁Dᵢ₃Sⱼ₁Dᵢ₄Sⱼ₁
ID tags constructed from an A_{D} alphabet of symbols selected at a minimum
mutual Euclidean distance of 3.825 where |A_{D}| = 256.

| ID Tag | Total Reads | Not Usable | Errors | Matches Temp. | Comp | Total |
|---|---|---|---|---|---|---|
| ID_F256eu_001 | 3397 | 1208 | 1525 | 333 | 331 | 664 |
| | | (35.6%) | (44.9%) | (9.8%) | (9.7%) | (19.5%) |
| ID_F256eu_001 | 4477 | 1514 | 1873 | 634 | 456 | 1090 |
| | | (33.8%) | (41.8%) | (14.2%) | (10.2%) | (24.3%) |
| ID_F256eu_001 | 4315 | 1466 | 2176 | 279 | 394 | 673 |
| | | (34%) | (50.4%) | (6.5%) | (9.1%) | (15.6%) |
| ID_F256eu_001 | 6026 | 1832 | 2780 | 798 | 616 | 1414 |
| | | (30.4%) | (46.1%) | (13.2%) | (10.2%) | (23.5%) |
| Euc. Dist | 18215 | 6020 | 8354 | 2044 | 1797 | 3841 |
| | | (33%) | (45.9%) | (11.2%) | (9.9%) | (21.1%) |

F16, Euclidean Distance, Spacer 1
ID_F16eu_001: S1/SEQ ID NO: 17/S1/SEQ ID NO: 18/S1/SEQ ID NO: 19/S1/SEQ ID NO: 20/S1
ID_F16eu_002: S1/SEQ ID NO: 21/S1/SEQ ID NO: 22/S1/SEQ ID NO: 23/S1/SEQ ID NO: 24/S1
ID_F16eu_003: S1/SEQ ID NO: 25/S1/SEQ ID NO: 26/S1/SEQ ID NO: 27/S1/SEQ ID NO: 28/S1
ID_F16eu_004: S1/SEQ ID NO: 29/S1/SEQ ID NO: 30/S1/SEQ ID NO: 31/S1/SEQ ID NO: 32/S1
ID_F16eu_005: S1/SEQ ID NO: 17/S1/SEQ ID NO: 21/S1/SEQ ID NO: 25/S1/SEQ ID NO: 29/S1
ID_F16eu_006: S1/SEQ ID NO: 20/S1/SEQ ID NO: 24/S1/SEQ ID NO: 28/S1/SEQ ID NO: 32/S1

F64, Euclidean Distance, Spacer 1
ID_F64eu_001: S1/SEQ ID NO: 146/S1/SEQ ID NO: 142/S1/SEQ ID NO: 124/S1/SEQ ID NO: 139/S1
ID_F64eu_002: S1/SEQ ID NO: 11I/S1/SEQ ID NO: 142/S1/SEQ ID NO: 124/S1/SEQ ID NO: 139/S1
ID_F64eu_003: S1/SEQ ID NO: 120/S1/SEQ ID NO: 134/S1/SEQ ID NO: 121/S1/SEQ ID NO: 146/S1
ID_F64eu_004: S1/SEQ ID NO: 142/S1/SEQ ID NO: 124/S1/SEQ ID NO: 139/S1/SEQ ID NO: 159/S1

F256, Euclidean Distance, Spacer 1
ID_F256eu_001: S1/SEQ ID NO: 441/S1/SEQ ID NO: 501/S1/SEQ ID NO: 616/S1/SEQ ID NO: 596/S1
ID_F256eu_002: S1/SEQ ID NO: 588/S1/SEQ ID NO: 501/S1/SEQ ID NO: 616/S1/SEQ ID NO: 596/S1
ID_F256eu_003: S1/SEQ ID NO: 535/S1/SEQ ID NO: 545/S1/SEQ ID NO: 421/S1/SEQ ID NO: 646/S1
ID_F256eu_004: S1/SEQ ID NO: 501/S1/SEQ ID NO: 616/S1/SEQ ID NO: 596/S1/SEQ ID NO: 488/S1

### Example 3: ID Tags that Include Spacers that Encode Data

To demonstrate the use of two alphabets to encode data, ID tags were assembled from alternating symbols from two different alphabets, A_{D} and A_{S}, where |A_{S}|=2 and C_{S} is the spacer configuration. As described previously, two alphabets may be used to increase the data rate r (bits nt⁻¹), distribute information across multiple different oligonucleotide fragments, or identify hidden information in an oligonucleotide watermark. In the following example, ID tags were constructed using the following alphabets:
A_{S}={S₁, S₂}→{0, 1}→{TTTTTTTT, AGAGAGAG}
A_{D}=a random set of symbols of length k_{D}=12 nt, where a symbol is denoted Dᵢ below

Specifically, the following ID tags that include spacer configurations C_{S} encoding data were constructed:
ID1=S₁DᵢS₁DᵢS₁DᵢS₁DᵢS₁, where C_{S}=00000
ID2=S₁DᵢS₁DᵢS₁DᵢS₂DᵢS₁, where C_{S}=00010
ID3=S₁DᵢS₁DᵢS₂DᵢS₂DᵢS₁, where C_{S}=00110
ID4=S₁DᵢS₁DᵢS₁DᵢS₁DᵢS₂, where C_{S}=00001
ID5=S₂DᵢS₁DᵢS₁DᵢS₁DᵢS₁, where C_{S}=10000
ID6=S₂DᵢS₂DᵢS₂DᵢS₂DᵢS₂, where C_{S}=11111
ID7=S₂DᵢS₂DᵢS₂DᵢS₁DᵢS₂, where C_{S}=11101
ID8=S₁DᵢS₁DᵢS₂DᵢS₁DᵢS₁, where C_{S}=00100
ID9=S₁DᵢS₂DᵢS₂DᵢS₂DᵢS₁, where C_{S}=01110
ID10=S₂DᵢS₂DᵢS₂DᵢS₂DᵢS₁, where C_{S}=11110

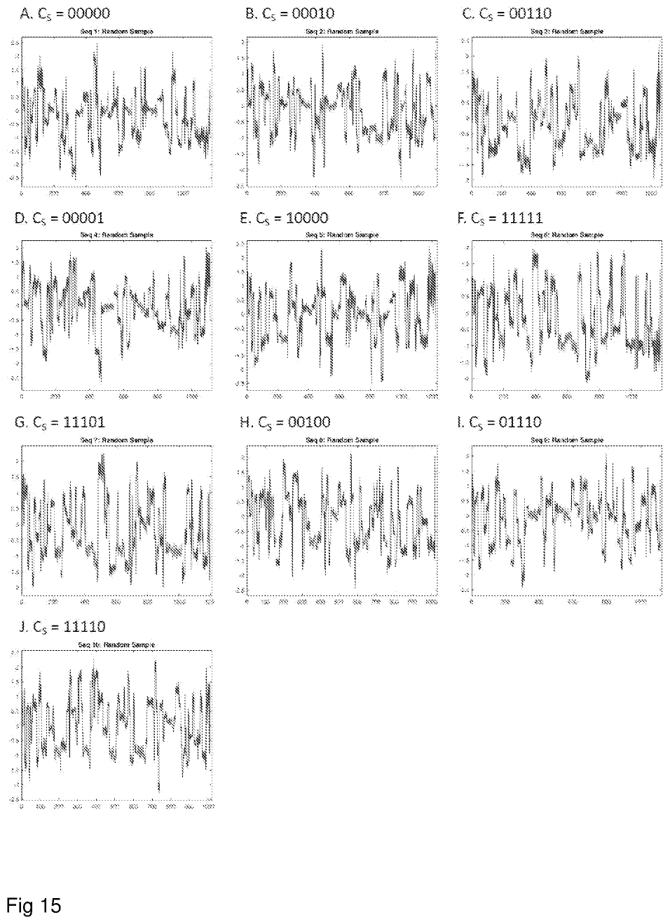

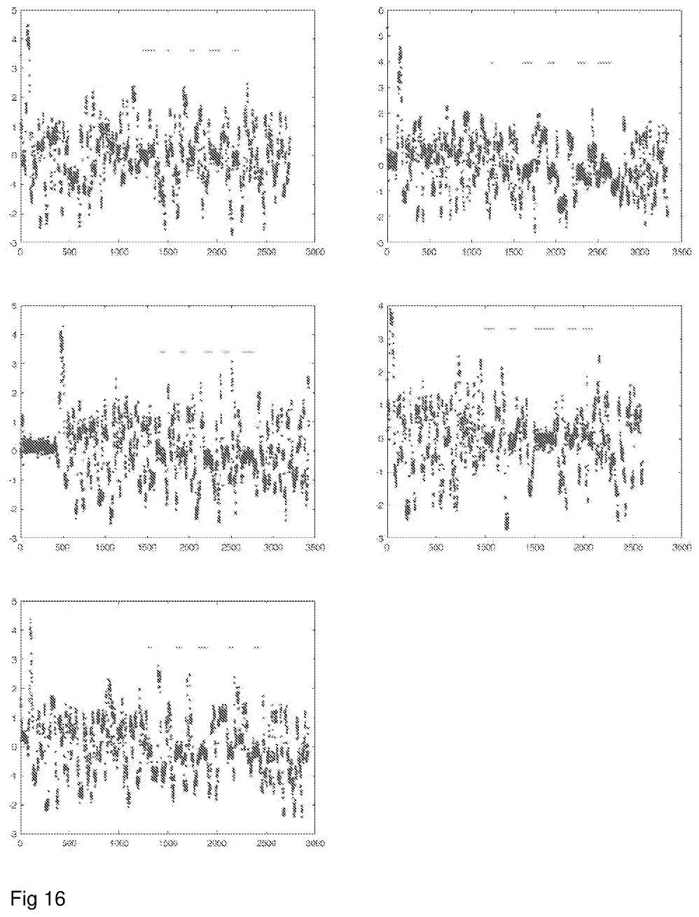

Analogue output from the ID tag sequences above (ID1-ID10) is given in FIG. 15. In all cases the spacer configurations could be easily identified and decoded. FIG. 16 also shows spacer detection on real nanopore output.

### Example 4: Unnatural Bases Improve Alphabet Design and Increase Data Rate r (Bits Nt-1)

To demonstrate the use of unnatural AEGIS modifications to improve symbol selection, four ID tags (ID_AEGIS_1-4) were manufactured with conventional DNA nucleotides from the set {A, C, G, T} and one or more AEGIS nucleotides from the set {P, Z, B, S}. These tags were manufacture by Firebird Biomolecular Science LLC, amplified with Phire Hotstart II DNA polymerase and ONT rapid attachment primers from the kit SQK-PBK004 in the presence of conventional free nucleotides only (dNTPs), and conventional and AEGIS free nucleotides (dXTPs). Samples were sequenced on an Oxford Nanopore MinION device using the SQK-PBK004 protocol and R9.4.1 flowcells.

ID_AG 1:
Primer-AAAPAAAPAACCGTAGTCAGCGAAAPAAAPAA-Primer
ID_AG 2:
Primer-AAAZAAAZAACCGTAGTCAGCGAAAZAAAZAA-Primer
ID_AG 3:
Primer-AAAGAAAGAAZAZAZAZAZAZAAAAGAAAGAA-Primer
ID_AG 3:
Primer-AAAGAAAGAAZZZAZZZAZZZAAAAGAAAGAA-Primer

Each sequence ID_AG_1-4 was amplified separately in the presence of dNTPs and dXTPs. When amplification was performed in the presence of dNTPs, any one of {A, C, G, or T} may amplified into position adjacent to an AEGIS base {Z, P, B, S} although bias towards C and T replacing Z, and G and A replacing P was observed.

The raw signals resulting from the sequencing runs were then clustered based on pair-wise DTW distance, and a consensus signal was generated for each primary cluster using DTW Barycenter Averaging (DBA). The regions of the consensus signals that are generated by the sequences containing the AEGIS bases were found by first locating the regions for the adjacent sub-sequences that do not contain the AEGIS bases, once more using DTW distances. FIG. 17 A-D show select average nanopore raw data generated by ID_AG_1-4 respectively. The left panels show ID_AG_1-4 amplified in the presence of dNTPs only (Ai-Di) and the right panels show ID_AG_1-4 amplified in the presence of dXTPs (Aii-Dii).

Table 10 gives the distance in DTW between sequences amplified in the presence of dNTPs and dXTPs. In all cases, tags amplified in the presence of dXTPs generated unique raw nanopore current signatures which were clearly detectable, in terms of DTW distance, from the same sequence amplified in the presence of dNTPs only. A visual inspection of FIG. 17, for example, also shows clearly different current signatures generated by the sub-sequences AAAPAAAPAA (Aii b), AAAZAAAZAA (Bii b) and AAAGAAAGAA (Ciib). These data demonstrate that AEGIS bases can be detected with nanopore sequencing and may be used to increase information rate, improve symbol selection, and improve decoding efficiency and reliability.

TABLE 10
Identification of raw nanopore current signatures
that that contain AEGIS bases

| | Region 1 | Region 2 | DTW distance |
|---|---|---|---|
| Tag | (+dNTPs) | (+dXTPs) | (normalised) |
| ID_AG_1 | FIG. 17 Ai(a) | FIG. 17 Aii(a) | 0.62 |
| | FIG. 17 Ai(b) | FIG. 17 Aii(b) | 0.29 |
| ID_AG_2 | FIG. 17 Bi(a) | FIG. 17 Bii(a) | 0.44 |
| | FIG. 17 Bi(b) | FIG. 17 Bii(b) | 0.35 |
| ID_AG_3 | FIG. 17 Ci(a) | FIG. 17 Cii(a) | 0.18 |
| ID_AG_4 | FIG. 17 Di(a) | FIG. 17 Dii(a) | 0.40 |

### Example Alphabets

Table 11-Table 16 below provide alphabet sequences, which relate to the examples above with the following relationship between the examples and the sequence listing:
F16abs relates to SEQ ID NOs: 1 to 16;
F16eu relates to SEQ ID NOs: 17 to 32;
F64abs relates to SEQ ID NOs: 33 to 96;
F64eu relates to SEQ ID NOs: 97 to 160;
F256abs relates to SEQ ID NOs: 161 to 416; and
F256eu relates to SEQ ID NOs: 417 to 672.

TABLE 11
provides an alphabet of 16 symbols selected by absolute distance

| | | | | | |
|---|---|---|---|---|---|
| SEQ ID | CGACGTGTACGC | SEQ ID | GGGAGGAGTCGC | SEQ ID | TCGGCCTGTGGG |
| NO: 1 | | NO: 7 | | NO: 13 | |
| SEQ ID | CGCCTACTCGGT | SEQ ID | GCCGATCGGACG | SEQ ID | GACGATCCTCGG |
| NO: 2 | | NO: 8 | | NO: 14 | |
| SEQ ID | GCCTGTAAGCGG | SEQ ID | GTGTCCGCTCTC | SEQ ID | GAGACTGGGCCC |
| NO: 3 | | NO: 9 | | NO: 15 | |
| SEQ ID | CCCAGAGGTTGG | SEQ ID | TCTCGCGGAGCT | SEQ ID | TCCTCTCTGCCG |
| NO: 4 | | NO: 10 | | NO: 16 | |
| SEQ ID | TGGATGGCGTCG | SEQ ID | CTGGGCCGAGAT | | |
| NO: 5 | | NO: 11 | | | |
| SEQ ID | GGGACTGATGGG | SEQ ID | GTCCGTTCGGGC | | |
| NO: 6 | | NO: 12 | | | |

TABLE 12
provides an alphabet of 16 symbols selected by Euclidean distance

| | | | | | |
|---|---|---|---|---|---|
| SEQ ID | CCCAGCTTAGGC | SEQ ID | CCGGAGTTACGG | SEQ ID | GTCCGCCTGAAC |
| NO: 17 | | NO: 23 | | NO: 29 | |
| SEQ ID | GGGCTTGCCCAT | SEQ ID | GCGCTCATAGCG | SEQ ID | CCGTGTGGATCC |
| NO: 18 | | NO: 24 | | NO: 30 | |
| SEQ ID | GAGGGTCTGTCG | SEQ ID | GGCAGTGAACGG | SEQ ID | GGGAGCGGGATC |
| NO: 19 | | NO: 25 | | NO: 31 | |
| SEQ ID | TCCTCTCTGCCG | SEQ ID | GGCAGGGTAGGC | SEQ ID | TCGTGGACTGCG |
| NO: 20 | | NO: 26 | | NO: 32 | |
| SEQ ID | CCGTGTGTTGGG | SEQ ID | CGGTCGTTCGCT | | |
| NO: 21 | | NO: 27 | | | |
| SEQ ID | CGGTTCTCTCCC | SEQ ID | CGTCATCTCGGG | | |
| NO: 22 | | NO: 28 | | | |

TABLE 13
provides an alphabet of 64 symbols selected by absolute distance

| | | | | | |
|---|---|---|---|---|---|
| SEQ ID | CGACGTGTACGC | SEQ ID | TGCGATGAGGCG | SEQ ID | GGCCTGCGAGTC |
| NO: 33 | | NO: 55 | | NO: 77 | |
| SEQ ID | GCCTGTAAGCGG | SEQ ID | CTGTCCAGTGGG | SEQ ID | TGGATGGCGTCG |
| NO: 34 | | NO: 56 | | NO: 78 | |
| SEQ ID | CCCAGAGGTTGG | SEQ ID | GCCTTGGTCGTG | SEQ ID | GGGACTGATGGG |
| NO: 35 | | NO: 57 | | NO: 79 | |
| SEQ ID | TGGTACGAGCCC | SEQ ID | TCGTGTCGCCAC | SEQ ID | CCCAGGATGGGT |
| NO: 36 | | NO: 58 | | NO: 80 | |
| SEQ ID | GGGATCAGCCGC | SEQ ID | GACGCGCCTGCG | SEQ ID | GCCGATCGGACG |
| NO: 37 | | NO: 59 | | NO: 81 | |
| SEQ ID | CCTGCGCACCAC | SEQ ID | TCAGCGGTCCCG | SEQ ID | GCTGGAGGCTAG |
| NO: 38 | | NO: 60 | | NO: 82 | |
| SEQ ID | GCCTACATGGGC | SEQ ID | CGCCTCTTTGCG | SEQ ID | GTGTCCGCTCTC |
| NO: 39 | | NO: 61 | | NO: 83 | |
| SEQ ID | CGTCACACAGGG | SEQ ID | CGCGCAAATGGC | SEQ ID | GATTCCCTCCGC |
| NO: 40 | | NO: 62 | | NO: 84 | |
| SEQ ID | GCCGATCTACCC | SEQ ID | GTTAGGCGGCGG | SEQ ID | GTGGACAGTCCG |
| NO: 41 | | NO: 63 | | NO: 85 | |
| SEQ ID | GGCAGTCGAGAG | SEQ ID | CCGCTCAGTGTC | SEQ ID | CGTTGTTGGCCG |
| NO: 42 | | NO: 64 | | NO: 86 | |
| SEQ ID | GTCATCGCCCTG | SEQ ID | GAGGGCAACGGT | SEQ ID | GTGTCCGTGACG |
| NO: 43 | | NO: 65 | | NO: 87 | |
| SEQ ID | CCGCGGGACTAT | SEQ ID | GCGTATCGTCGC | SEQ ID | TCGGGCGCCGAG |
| NO: 44 | | NO: 66 | | NO: 88 | |
| SEQ ID | CCGAAGGGCAGT | SEQ ID | CGGATCGAACGG | SEQ ID | GTCCGTTCGGGC |
| NO: 45 | | NO: 67 | | NO: 89 | |
| SEQ ID | CGTCCCAGATCG | SEQ ID | GCGTGCGACGAC | SEQ ID | GCCCTCTCGTCG |
| NO: 46 | | NO: 68 | | NO: 90 | |
| SEQ ID | GGATTCCTGCGG | SEQ ID | GGCAAGAGGGCT | SEQ ID | CTCGTCGTCTCG |
| NO: 47 | | NO: 69 | | NO: 91 | |
| SEQ ID | GCAGTGTCAGGG | SEQ ID | GAGTGGCGTCGT | SEQ ID | CCGTGTGTTGGG |
| NO: 48 | | NO: 70 | | NO: 92 | |
| SEQ ID | GCCCAACGTTCC | SEQ ID | CCGCAGCTAGAG | SEQ ID | CGGTTCTCTCCC |
| NO: 49 | | NO: 71 | | NO: 93 | |
| SEQ ID | GGAGGGCATCTG | SEQ ID | TCCCATCAGCGG | SEQ ID | GCGGTGGATTGG |
| NO: 50 | | NO: 72 | | NO: 94 | |
| SEQ ID | TCGAACCGTCGC | SEQ ID | CGTGGGTTGGAC | SEQ ID | CGGTGGTCCATC |
| NO: 51 | | NO: 73 | | NO: 95 | |
| SEQ ID | CGAAGACCCTCG | SEQ ID | TGGGTACCGCGG | SEQ ID | CCCTCAGTTCCG |
| NO: 52 | | NO: 74 | | NO: 96 | |
| SEQ ID | GTCCACGAACGG | SEQ ID | GGGCTTCTGCCT | | |
| NO: 53 | | NO: 75 | | | |
| SEQ ID | CCGTGTGGATCC | SEQ ID | CGCCTACTCGGT | | |
| NO: 54 | | NO: 76 | | | |

TABLE 14
provides an alphabet of 64 symbols selected by Euclidean distance

| | | | | | |
|---|---|---|---|---|---|
| SEQ ID | CCCAGCTTAGGC | SEQ ID | GCCTCAATGCCC | SEQ ID | GAGGGTCTGTCG |
| NO: 97 | | NO: 119 | | NO: 141 | |
| SEQ ID | CCAAGTGCGCAC | SEQ ID | GGGCTTGCCCAT | SEQ ID | GGAGGATGGCGG |
| NO: 98 | | NO: 120 | | NO: 142 | |
| SEQ ID | TCCTCTCTGCCG | SEQ ID | GACGCAGCCCTG | SEQ ID | CCGGAGTTACGG |
| NO: 99 | | NO: 121 | | NO: 143 | |
| SEQ ID | CCGTGTGTTGGG | SEQ ID | CGGTTCTCTCCC | SEQ ID | GTGTCCGCTCTC |
| NO: 100 | | NO: 122 | | NO: 144 | |
| SEQ ID | GGCAGTGAACGG | SEQ ID | TCGGCCTGTGGG | SEQ ID | TCAGCGGTCCCG |
| NO: 101 | | NO: 123 | | NO: 145 | |
| SEQ ID | GCGACCATCTCG | SEQ ID | CCCTACCCTCCT | SEQ ID | GGGAGTTTGGCC |
| NO: 102 | | NO: 124 | | NO: 146 | |
| SEQ ID | CGAAGTGGCGTC | SEQ ID | CCGCAGCTAGAG | SEQ ID | TGCCGTCGGGCC |
| NO: 103 | | NO: 125 | | NO: 147 | |
| SEQ ID | GCTCGTCCCTGT | SEQ ID | GGGCACAAGTGG | SEQ ID | CGGTCGTTCGCT |
| NO: 104 | | NO: 126 | | NO: 148 | |
| SEQ ID | GGCAGGGTAGGC | SEQ ID | GCCGTGAGTCTG | SEQ ID | GCCTCGTGTGTG |
| NO: 105 | | NO: 127 | | NO: 149 | |
| SEQ ID | GGGAGCCAAGTC | SEQ ID | TCGGTGGTGTGC | SEQ ID | TGGTGGGAAGCG |
| NO: 106 | | NO: 128 | | NO: 150 | |
| SEQ ID | GTCGGGAAGGCT | SEQ ID | GATGGAGCGGTG | SEQ ID | GTGGTCCGTGTC |
| NO: 107 | | NO: 129 | | NO: 151 | |
| SEQ ID | CGTCCTTCTCCG | SEQ ID | GTCCGCCTGAAC | SEQ ID | CTCGGAATGGCG |
| NO: 108 | | NO: 130 | | NO: 152 | |
| SEQ ID | GCGTCGATTGGG | SEQ ID | GTCATCGCCCTG | SEQ ID | GCGGACACGGTT |
| NO: 109 | | NO: 131 | | NO: 153 | |
| SEQ ID | GTCCACGAACGG | SEQ ID | CGCCCTAATCGG | SEQ ID | CGGTCATGGACC |
| NO: 110 | | NO: 132 | | NO: 154 | |
| SEQ ID | GGGAGGAGTCGC | SEQ ID | GATTCCCTCCGC | SEQ ID | CGTGCTCTCCGT |
| NO: 111 | | NO: 133 | | NO: 155 | |
| SEQ ID | GCCCTCTCGTCG | SEQ ID | GCGACGGCTAAC | SEQ ID | CGAAGACCCTCG |
| NO: 112 | | NO: 134 | | NO: 156 | |
| SEQ ID | CGTGGGTTGGAC | SEQ ID | CACGGCCTCGTT | SEQ ID | TCGGTCGCTCCG |
| NO: 113 | | NO: 135 | | NO: 157 | |
| SEQ ID | GACGATCCTCGG | SEQ ID | CGGGAGAAACCC | SEQ ID | GCCTCTAGGAGG |
| NO: 114 | | NO: 136 | | NO: 158 | |
| SEQ ID | GTCGGCGTTGAC | SEQ ID | CCCTCAGTTCCG | SEQ ID | GACGTTCGAGGG |
| NO: 115 | | NO: 137 | | NO: 159 | |
| SEQ ID | CGGTGGTCCATC | SEQ ID | CGTTGTTGGCCG | SEQ ID | CCGTTCGCGTTG |
| NO: 116 | | NO: 138 | | NO: 160 | |
| SEQ ID | GCGTAACGCGTG | SEQ ID | GGGTTTCCAGGG | | |
| NO: 117 | | NO: 139 | | | |
| SEQ ID | TCCTCGACAGCC | SEQ ID | TCGAACCGTCGC | | |
| NO: 118 | | NO: 140 | | | |

TABLE 15
provides an alphabet of 256 symbols selected by absolute distance

| | | | | | |
|---|---|---|---|---|---|
| SEQ ID | AAAAGGTGTG | SEQ ID | GGATGGATAA | SEQ ID | TATAAGGTGG |
| NO: 161 | | NO: 247 | | NO: 333 | |
| SEQ ID | AAAGTGGGTA | SEQ ID | GGATTAAAGG | SEQ ID | TATAGGTGAG |
| NO: 162 | | NO: 248 | | NO: 334 | |
| SEQ ID | AAGAAGAAGG | SEQ ID | GGATTGGATG | SEQ ID | TATGGATAGG |
| NO: 163 | | NO: 249 | | NO: 335 | |
| SEQ ID | AAGAGGGTAG | SEQ ID | GGATTGTGGA | SEQ ID | TATGGTGTGG |
| NO: 164 | | NO: 250 | | NO: 336 | |
| SEQ ID | AAGAGGTTGT | SEQ ID | GGATTTGTGT | SEQ ID | TATGGTTGGT |
| NO: 165 | | NO: 251 | | NO: 337 | |
| SEQ ID | AAGATATGGG | SEQ ID | GGGAAAAGTT | SEQ ID | TATGTAGGGA |
| NO: 166 | | NO: 252 | | NO: 338 | |
| SEQ ID | AAGGTTTGGA | SEQ ID | GGGAAATTTG | SEQ ID | TATGTGGGTT |
| NO: 167 | | NO: 253 | | NO: 339 | |
| SEQ ID | AAGTTGGAAG | SEQ ID | GGGAAGAAAA | SEQ ID | TATTTGGGAG |
| NO: 168 | | NO: 254 | | NO: 340 | |
| SEQ ID | AAGTTGGAGT | SEQ ID | GGGAAGATAG | SEQ ID | TATTTGGGTG |
| NO: 169 | | NO: 255 | | NO: 341 | |
| SEQ ID | AAGTTGTGTG | SEQ ID | GGTAAAGAAG | SEQ ID | TATTTGTGGG |
| NO: 170 | | NO: 256 | | NO: 342 | |
| SEQ ID | AAGTTTGAGG | SEQ ID | GGTAAAGGTT | SEQ ID | TGAAAGGTGT |
| NO: 171 | | NO: 257 | | NO: 343 | |
| SEQ ID | AATAGGTGTG | SEQ ID | GGTAGAATAG | SEQ ID | TGAAGGTATG |
| NO: 172 | | NO: 258 | | NO: 344 | |
| SEQ ID | AATATGGTGG | SEQ ID | GGTAGGTTAA | SEQ ID | TGAAGGTTGG |
| NO: 173 | | NO: 259 | | NO: 345 | |
| SEQ ID | AATGGAGGGT | SEQ ID | GGTAGGTTTG | SEQ ID | TGAATAGGTG |
| NO: 174 | | NO: 260 | | NO: 346 | |
| SEQ ID | AATTGGAGGG | SEQ ID | GGTAGTTGGA | SEQ ID | TGAATGGAGA |
| NO: 175 | | NO: 261 | | NO: 347 | |
| SEQ ID | AATTGGATGG | SEQ ID | GGTATGGAAA | SEQ ID | TGAGGATGGG |
| NO: 176 | | NO: 262 | | NO: 348 | |
| SEQ ID | AATTTGGGTG | SEQ ID | GGTATGGTTT | SEQ ID | TGAGGTTAGA |
| NO: 177 | | NO: 263 | | NO: 349 | |
| SEQ ID | AATTTGTGGG | SEQ ID | GGTGTAAAGA | SEQ ID | TGAGGTTTGT |
| NO: 178 | | NO: 264 | | NO: 350 | |
| SEQ ID | AGAAAAGGTG | SEQ ID | GGTGTAGTTG | SEQ ID | TGAGTIGTGA |
| NO: 179 | | NO: 265 | | NO: 351 | |
| SEQ ID | AGAAGAGGGT | SEQ ID | GGTTAAAGGT | SEQ ID | TGGAAAGGGA |
| NO: 180 | | NO: 266 | | NO: 352 | |
| SEQ ID | AGAGTATGGA | SEQ ID | GGTTAGGTTT | SEQ ID | TGGAAGGTTT |
| NO: 181 | | NO: 267 | | NO: 353 | |
| SEQ ID | AGGAAAGTGT | SEQ ID | GGTTATATGG | SEQ ID | TGGAAGTTGT |
| NO: 182 | | NO: 268 | | NO: 354 | |
| SEQ ID | AGGAATGGAA | SEQ ID | GGTTATGGAG | SEQ ID | TGGAATAGGT |
| NO: 183 | | NO: 269 | | NO: 355 | |
| SEQ ID | AGGGAAGTTA | SEQ ID | GGTTGAATGG | SEQ ID | TGGATAGGTT |
| NO: 184 | | NO: 270 | | NO: 356 | |
| SEQ ID | AGGGTATATG | SEQ ID | GGTTGATAAG | SEQ ID | TGGATATGGA |
| NO: 185 | | NO: 271 | | NO: 357 | |
| SEQ ID | AGGGTGGTTA | SEQ ID | GGTTGGTTAG | SEQ ID | TGGGAAATGG |
| NO: 186 | | NO: 272 | | NO: 358 | |
| SEQ ID | AGGTGGGTGT | SEQ ID | GGTTGTATGT | SEQ ID | TGGGAAGTTA |
| NO: 187 | | NO: 273 | | NO: 359 | |
| SEQ ID | AGGTGTATGG | SEQ ID | GGTTGTGGGT | SEQ ID | TGGGAATAAG |
| NO: 188 | | NO: 274 | | NO: 360 | |
| SEQ ID | AGGTTATAGG | SEQ ID | GGTTGTGTAG | SEQ ID | TGGGAATTTG |
| NO: 189 | | NO: 275 | | NO: 361 | |
| SEQ ID | AGGTTGAGAA | SEQ ID | GGTTTGGAAG | SEQ ID | TGGGTAGATA |
| NO: 190 | | NO: 276 | | NO: 362 | |
| SEQ ID | AGGTTGGATT | SEQ ID | GGTTTGTATG | SEQ ID | TGGGTAGTTA |
| NO: 191 | | NO: 277 | | NO: 363 | |
| SEQ ID | AGTAAGGTTG | SEQ ID | GGTTTTGGTA | SEQ ID | TGGGTATAGG |
| NO: 192 | | NO: 278 | | NO: 364 | |
| SEQ ID | AGTATGGAGT | SEQ ID | GTAAAGGGTA | SEQ ID | TGGGTGGTTG |
| NO: 193 | | NO: 279 | | NO: 365 | |
| SEQ ID | AGTATGGTGT | SEQ ID | GTAAGGATAG | SEQ ID | TGGTATGTAG |
| NO: 194 | | NO: 280 | | NO: 366 | |
| SEQ ID | AGTTAGGTAG | SEQ ID | GTAGATATGG | SEQ ID | TGGTGTAGAA |
| NO: 195 | | NO: 281 | | NO: 367 | |
| SEQ ID | AGTTGGTGTA | SEQ ID | GTAGATTAGG | SEQ ID | TGGTGTATGT |
| NO: 196 | | NO: 282 | | NO: 368 | |
| SEQ ID | AGTTGGTTTG | SEQ ID | GTAGGTATGT | SEQ ID | TGGTGTGGTT |
| NO: 197 | | NO: 283 | | NO: 369 | |
| SEQ ID | AGTTTGGGTT | SEQ ID | GTAGGTGAAA | SEQ ID | TGGTTAATGG |
| NO: 198 | | NO: 284 | | NO: 370 | |
| SEQ ID | ATAAGGTAGG | SEQ ID | GTAGGTTATG | SEQ ID | TGGTTGAAAG |
| NO: 199 | | NO: 285 | | NO: 371 | |
| SEQ ID | ATAGGTTGAG | SEQ ID | GTAGTTTGGT | SEQ ID | TGGTTGGGTA |
| NO: 200 | | NO: 286 | | NO: 372 | |
| SEQ ID | ATATGGAGGG | SEQ ID | GTATAGAAGG | SEQ ID | TGGTTGGTTT |
| NO: 201 | | NO: 287 | | NO: 373 | |
| SEQ ID | ATGGAATGGA | SEQ ID | GTATAGGTGG | SEQ ID | TGGTTGTAGT |
| NO: 202 | | NO: 288 | | NO: 374 | |
| SEQ ID | ATTTTGGAGG | SEQ ID | GTATGAGGTT | SEQ ID | TGGTTTGTGG |
| NO: 203 | | NO: 289 | | NO: 375 | |
| SEQ ID | GAAAAGTGGA | SEQ ID | GTATGGTATG | SEQ ID | TGTAAGGGTA |
| NO: 204 | | NO: 290 | | NO: 376 | |
| SEQ ID | GAAAGAATGG | SEQ ID | GTTAAAGGAG | SEQ ID | TGTAAGGTTG |
| NO: 205 | | NO: 291 | | NO: 377 | |
| SEQ ID | GAAAGGTTGG | SEQ ID | GTTAAAGTGG | SEQ ID | TGTAGTTGGA |
| NO: 206 | | NO: 292 | | NO: 378 | |
| SEQ ID | GAAATGGAAG | SEQ ID | GTTAAGGTGT | SEQ ID | TGTAGTTGTG |
| NO: 207 | | NO: 293 | | NO: 379 | |
| SEQ ID | GAAGGATATG | SEQ ID | GTTAGTTGTG | SEQ ID | TGTATAGGGT |
| NO: 208 | | NO: 294 | | NO: 380 | |
| SEQ ID | GAAGGTAGAA | SEQ ID | GTTATATGGG | SEQ ID | TGTATGGAAG |
| NO: 209 | | NO: 295 | | NO: 381 | |
| SEQ ID | GAAGTAAAGG | SEQ ID | GTTATGGAAG | SEQ ID | TGTGAAAAGG |
| NO: 210 | | NO: 296 | | NO: 382 | |
| SEQ ID | GAAGTTATGG | SEQ ID | GTTATGGATG | SEQ ID | TGTGAGGTTT |
| NO: 211 | | NO: 297 | | NO: 383 | |
| SEQ ID | GAAGTTGGGA | SEQ ID | GTTATGGTTG | SEQ ID | TGTGGGAAGA |
| NO: 212 | | NO: 298 | | NO: 384 | |
| SEQ ID | GAATAGGTGG | SEQ ID | GTTGAGAAGG | SEQ ID | TGTGGGATGG |
| NO: 213 | | NO: 299 | | NO: 385 | |
| SEQ ID | GAGAAAGGAA | SEQ ID | GTTGGAAGAA | SEQ ID | TGTGGGTGTA |
| NO: 214 | | NO: 300 | | NO: 386 | |
| SEQ ID | GAGGAAGTGG | SEQ ID | GTTGGAAGTT | SEQ ID | TGTGGTATAG |
| NO: 215 | | NO: 301 | | NO: 387 | |
| SEQ ID | GAGGGTATAA | SEQ ID | GTTGGAATAG | SEQ ID | TGTGGTTTTG |
| NO: 216 | | NO: 302 | | NO: 388 | |
| SEQ ID | GAGGTAATAG | SEQ ID | GTTGGATATG | SEQ ID | TTAAAGGTGG |
| NO: 217 | | NO: 303 | | NO: 389 | |
| SEQ ID | GAGTTTTGGG | SEQ ID | GTTGGGTGAG | SEQ ID | TTAAGGTGTG |
| NO: 218 | | NO: 304 | | NO: 390 | |
| SEQ ID | GATAGGTAGA | SEQ ID | GTTGGTTGGG | SEQ ID | TTAATGGAGG |
| NO: 219 | | NO: 305 | | NO: 391 | |
| SEQ ID | GATAGGTATG | SEQ ID | GTTGTAAAGG | SEQ ID | TTAGGGTGTA |
| NO: 220 | | NO: 306 | | NO: 392 | |
| SEQ ID | GATAGGTTGT | SEQ ID | GTTGTATGGA | SEQ ID | TTAGGTGGGT |
| NO: 221 | | NO: 307 | | NO: 393 | |
| SEQ ID | GATATAGGGT | SEQ ID | GTTGTGAGAA | SEQ ID | TTAGGTTGGG |
| NO: 222 | | NO: 308 | | NO: 394 | |
| SEQ ID | GATATGGAGA | SEQ ID | GTTGTGGGTG | SEQ ID | TTATGTAGGG |
| NO: 223 | | NO: 309 | | NO: 395 | |
| SEQ ID | GATATGGTTG | SEQ ID | GTTGTGGTTA | SEQ ID | TTGAGGAAGA |
| NO: 224 | | NO: 310 | | NO: 396 | |
| SEQ ID | GATGGAAGGG | SEQ ID | GTTGTGTATG | SEQ ID | TTGGAGGGTA |
| NO: 225 | | NO: 311 | | NO: 397 | |
| SEQ ID | GATGGAATTG | SEQ ID | GTTTAGTTGG | SEQ ID | TTGGGTAGTT |
| NO: 226 | | NO: 312 | | NO: 398 | |
| SEQ ID | GATTGGGAAG | SEQ ID | GTTTGATAGG | SEQ ID | TTGGGTGGGA |
| NO: 227 | | NO: 313 | | NO: 399 | |
| SEQ ID | GATTGGGTGG | SEQ ID | GTTTGGTTGT | SEQ ID | TTGGGTGTGG |
| NO: 228 | | NO: 314 | | NO: 400 | |
| SEQ ID | GATTGTGTGA | SEQ ID | GTTTGTGTGG | SEQ ID | TTGGTTGGTT |
| NO: 229 | | NO: 315 | | NO: 401 | |
| SEQ ID | GATTTAAGGG | SEQ ID | GTTTTGAGGA | SEQ ID | TTGGTTGTAG |
| NO: 230 | | NO: 316 | | NO: 402 | |
| SEQ ID | GATTTGGGTA | SEQ ID | GTTTTGGAGT | SEQ ID | TTGGTTGTGT |
| NO: 231 | | NO: 317 | | NO: 403 | |
| SEQ ID | GATTTTGTGG | SEQ ID | GTTTTGTGGA | SEQ ID | TTGGTTTGGA |
| NO: 232 | | NO: 318 | | NO: 404 | |
| SEQ ID | GGAAAGGTTT | SEQ ID | TAAAGAGGGT | SEQ ID | TTGTAGGGAA |
| NO: 233 | | NO: 319 | | NO: 405 | |
| SEQ ID | GGAAGAGGAG | SEQ ID | TAAAGGATGG | SEQ ID | TTGTATGGAG |
| NO: 234 | | NO: 320 | | NO: 406 | |
| SEQ ID | GGAAGGTTAG | SEQ ID | TAAGAGAAGG | SEQ ID | TTGTATGTGG |
| NO: 235 | | NO: 321 | | NO: 407 | |
| SEQ ID | GGAAGTATGT | SEQ ID | TAAGGGTAGT | SEQ ID | TTGTGGGTAG |
| NO: 236 | | NO: 322 | | NO: 408 | |
| SEQ ID | GGAAGTTGGT | SEQ ID | TAAGGGTGGA | SEQ ID | TTGTGGTTGT |
| NO: 237 | | NO: 323 | | NO: 409 | |
| SEQ ID | GGAATAGGGT | SEQ ID | TAAGTATGGG | SEQ ID | TTGTGTGGGT |
| NO: 238 | | NO: 324 | | NO: 410 | |
| SEQ ID | GGAGGATAAA | SEQ ID | TAAGTTGGGT | SEQ ID | TTTAGGGTAG |
| NO: 239 | | NO: 325 | | NO: 411 | |
| SEQ ID | GGAGGTTGTG | SEQ ID | TAGAAAGGTG | SEQ ID | TTTATGGTGG |
| NO: 240 | | NO: 326 | | NO: 412 | |
| SEQ ID | GGAGGTTTTA | SEQ ID | TAGGTAGAAG | SEQ ID | TTTGAGGTTG |
| NO: 241 | | NO: 327 | | NO: 413 | |
| SEQ ID | GGAGTAGTTT | SEQ ID | TAGGTGTATG | SEQ ID | TTTGGAAAGG |
| NO: 242 | | NO: 328 | | NO: 414 | |
| SEQ ID | GGATATGGTT | SEQ ID | TAGGTTGGTT | SEQ ID | TTTGGGTAGT |
| NO: 243 | | NO: 329 | | NO: 415 | |
| SEQ ID | GGATATGTAG | SEQ ID | TAGGTTTGGA | SEQ ID | TTTGGTATGG |
| NO: 244 | | NO: 330 | | NO: 416 | |
| SEQ ID | GGATGGAAGA | SEQ ID | TAGTTGGAGA | | |
| NO: 245 | | NO: 331 | | | |
| SEQ ID | GGATGGAATT | SEQ ID | TAGTTTTGGG | | |
| NO: 246 | | NO: 332 | | | |

TABLE 16
provides an alphabet of 256 symbols selected by Euclidean distance

| | | | | | |
|---|---|---|---|---|---|
| SEQ ID | AAAAGGATGG | SEQ ID | GGATATGGTA | SEQ ID | TATAGGTGTG |
| NO: 417 | | NO: 503 | | NO: 589 | |
| SEQ ID | AAAGTGGGTT | SEQ ID | GGATATGTAG | SEQ ID | TATATGAGGG |
| NO: 420 | | NO: 504 | | NO: 590 | |
| SEQ ID | AAATAGGTGG | SEQ ID | GGATGGAAAA | SEQ ID | TATGGAAGAG |
| NO: 419 | | NO: 505 | | NO: 591 | |
| SEQ ID | AAATTGTGGG | SEQ ID | GGATGGATAT | SEQ ID | TATGGTGGTT |
| NO: 420 | | NO: 506 | | NO: 592 | |
| SEQ ID | AAGAAGGGTA | SEQ ID | GGGAAATGGA | SEQ ID | TATGGTGTGA |
| NO: 421 | | NO: 507 | | NO: 593 | |
| SEQ ID | AAGGGAAAGG | SEQ ID | GGGAAGAAAT | SEQ ID | TATGGTTAGG |
| NO: 422 | | NO: 508 | | NO: 594 | |
| SEQ ID | AAGGGTGAAT | SEQ ID | GGGAAGGATT | SEQ ID | TATGTGGTTG |
| NO: 423 | | NO: 509 | | NO: 595 | |
| SEQ ID | AAGGTATGTG | SEQ ID | GGGTAAGTTA | SEQ ID | TATGTGTGGT |
| NO: 424 | | NO: 510 | | NO: 596 | |
| SEQ ID | AAGGTTGAGA | SEQ ID | GGGTGTATAA | SEQ ID | TATTGTGGGA |
| NO: 425 | | NO: 511 | | NO: 597 | |
| SEQ ID | AAGGTTTGGG | SEQ ID | GGTAAAGGAT | SEQ ID | TATTTGGAGG |
| NO: 426 | | NO: 512 | | NO: 598 | |
| SEQ ID | AAGTTGGGTA | SEQ ID | GGTAGAATAG | SEQ ID | TGAAGAGGAT |
| NO: 427 | | NO: 513 | | NO: 599 | |
| SEQ ID | AATATGTGGG | SEQ ID | GGTAGTTGAA | SEQ ID | TGAAGAGGTG |
| NO: 428 | | NO: 514 | | NO: 600 | |
| SEQ ID | AATTGGTTGG | SEQ ID | GGTATAAAGG | SEQ ID | TGAAGGATAG |
| NO: 429 | | NO: 515 | | NO: 601 | |
| SEQ ID | AGAAAATGGG | SEQ ID | GGTATGGATA | SEQ ID | TGAGAGGTTA |
| NO: 430 | | NO: 516 | | NO: 602 | |
| SEQ ID | AGAAGGTTGG | SEQ ID | GGTGAATAGG | SEQ ID | TGAGGAAGGG |
| NO: 431 | | NO: 517 | | NO: 603 | |
| SEQ ID | AGAGAGGAAA | SEQ ID | GGTGGGTAAT | SEQ ID | TGAGGTTATG |
| NO: 432 | | NO: 518 | | NO: 604 | |
| SEQ ID | AGAGGTGTAT | SEQ ID | GGTGTATGGG | SEQ ID | TGAGGTTGAT |
| NO: 433 | | NO: 519 | | NO: 605 | |
| SEQ ID | AGAGGTTGTG | SEQ ID | GGTGTGAAAA | SEQ ID | TGGAAGGAAA |
| NO: 434 | | NO: 520 | | NO: 606 | |
| SEQ ID | AGATAGGGTA | SEQ ID | GGTTAAAGGT | SEQ ID | TGGAAGGTAT |
| NO: 435 | | NO: 521 | | NO: 607 | |
| SEQ ID | AGATATGGTG | SEQ ID | GGTTGGATAG | SEQ ID | TGGAAGTAGA |
| NO: 436 | | NO: 522 | | NO: 608 | |
| SEQ ID | AGGAATTGGA | SEQ ID | GGTTGGTTAT | SEQ ID | TGGAATAAGG |
| NO: 437 | | NO: 523 | | NO: 609 | |
| SEQ ID | AGGATATGGA | SEQ ID | GGTTGTAATG | SEQ ID | TGGAATATGG |
| NO: 438 | | NO: 524 | | NO: 610 | |
| SEQ ID | AGGGAATAAG | SEQ ID | GGTTGTATAG | SEQ ID | TGGATATAGG |
| NO: 439 | | NO: 525 | | NO: 611 | |
| SEQ ID | AGGGTATAGT | SEQ ID | GGTTGTGAGG | SEQ ID | TGGATATGGT |
| NO: 440 | | NO: 526 | | NO: 612 | |
| SEQ ID | AGGTAGTTGT | SEQ ID | GGTTGTGTAT | SEQ ID | TGGGAAAGTA |
| NO: 441 | | NO: 527 | | NO: 613 | |
| SEQ ID | AGGTATATGG | SEQ ID | GGTTTGGAAA | SEQ ID | TGGGAAGTGG |
| NO: 442 | | NO: 528 | | NO: 614 | |
| SEQ ID | AGGTGAAAGG | SEQ ID | GGTTTGTAGT | SEQ ID | TGGGAAGTTT |
| NO: 443 | | NO: 529 | | NO: 615 | |
| SEQ ID | AGGTGTAAAG | SEQ ID | GGTTTTATGG | SEQ ID | TGGGAATATG |
| NO: 444 | | NO: 530 | | NO: 616 | |
| SEQ ID | AGGTGTAGTT | SEQ ID | GGTTTTGGTG | SEQ ID | TGGGTAGTTA |
| NO: 445 | | NO: 531 | | NO: 617 | |
| SEQ ID | AGGTTATTGG | SEQ ID | GTAAGATTGG | SEQ ID | TGGGTATGTA |
| NO: 446 | | NO: 532 | | NO: 618 | |
| SEQ ID | AGGTTGGTAA | SEQ ID | GTAAGGTATG | SEQ ID | TGGGTGAGAT |
| NO: 447 | | NO: 533 | | NO: 619 | |
| SEQ ID | AGTAAGGAAG | SEQ ID | GTAGAAAGGA | SEQ ID | TGGGTGTATT |
| NO: 448 | | NO: 534 | | NO: 620 | |
| SEQ ID | AGTAAGGTGT | SEQ ID | GTAGGTAGAT | SEQ ID | TGGTATGGAA |
| NO: 449 | | NO: 535 | | NO: 621 | |
| SEQ ID | AGTAGGTGGG | SEQ ID | GTAGGTGTAT | SEQ ID | TGGTATGGAT |
| NO: 450 | | NO: 536 | | NO: 622 | |
| SEQ ID | AGTATAGGGT | SEQ ID | GTAGGTTAAG | SEQ ID | TGGTGTGTAG |
| NO: 451 | | NO: 537 | | NO: 623 | |
| SEQ ID | AGTTAAAGGG | SEQ ID | GTAGGTTTTG | SEQ ID | TGGTGTGTAT |
| NO: 452 | | NO: 538 | | NO: 624 | |
| SEQ ID | AGTTGGAAGA | SEQ ID | GTATAGGTGT | SEQ ID | TGGTTGATAG |
| NO: 453 | | NO: 539 | | NO: 625 | |
| SEQ ID | AGTTGTGGGA | SEQ ID | GTATAGTTGG | SEQ ID | TGGTTGGTAT |
| NO: 454 | | NO: 540 | | NO: 626 | |
| SEQ ID | AGTTGTGTGG | SEQ ID | GTATATGGAG | SEQ ID | TGGTTGTAGT |
| NO: 455 | | NO: 541 | | NO: 627 | |
| SEQ ID | AGTTTATGGG | SEQ ID | GTATATGTGG | SEQ ID | TGGTTTAGAG |
| NO: 456 | | NO: 542 | | NO: 628 | |
| SEQ ID | AGTTTGGGAG | SEQ ID | GTATGAGGAT | SEQ ID | TGGTTTGGTT |
| NO: 457 | | NO: 543 | | NO: 629 | |
| SEQ ID | ATAGGTAGGG | SEQ ID | GTATGGAAAG | SEQ ID | TGGTTTGTGG |
| NO: 458 | | NO: 544 | | NO: 630 | |
| SEQ ID | ATAGGTGTGG | SEQ ID | GTATGGATAG | SEQ ID | TGTAAGGGTA |
| NO: 459 | | NO: 545 | | NO: 631 | |
| SEQ ID | ATAGGTTGGT | SEQ ID | GTTAATAGGG | SEQ ID | TGTAAGTGGG |
| NO: 460 | | NO: 546 | | NO: 632 | |
| SEQ ID | ATATGAAGGG | SEQ ID | GTTAGGTGAA | SEQ ID | TGTAGGTTGG |
| NO: 461 | | NO: 547 | | NO: 633 | |
| SEQ ID | ATGGAATGGA | SEQ ID | GTTAGTTGTG | SEQ ID | TGTAGTTGTG |
| NO: 462 | | NO: 548 | | NO: 634 | |
| SEQ ID | ATGGAGGGTA | SEQ ID | GTTATGGAGA | SEQ ID | TGTATAGGTG |
| NO: 463 | | NO: 549 | | NO: 635 | |
| SEQ ID | ATTTTGGAGG | SEQ ID | GTTATGGTTG | SEQ ID | TGTATATGGG |
| NO: 464 | | NO: 550 | | NO: 636 | |
| SEQ ID | GAAAAGGTTG | SEQ ID | GTTGAGGAAA | SEQ ID | TGTGAGAAGG |
| NO: 465 | | NO: 551 | | NO: 637 | |
| SEQ ID | GAAGAAAGGA | SEQ ID | GTTGGAAGAT | SEQ ID | TGTGAGGTTT |
| NO: 466 | | NO: 552 | | NO: 638 | |
| SEQ ID | GAAGGGTATT | SEQ ID | GTTGGAATAG | SEQ ID | TGTGGGTAAA |
| NO: 467 | | NO: 553 | | NO: 639 | |
| SEQ ID | GAAGTGGGTG | SEQ ID | GTTGGATAGG | SEQ ID | TGTGGGTATT |
| NO: 468 | | NO: 554 | | NO: 640 | |
| SEQ ID | GAAGTTGTGT | SEQ ID | GTTGGGTATA | SEQ ID | TGTGGTATGG |
| NO: 469 | | NO: 555 | | NO: 641 | |
| SEQ ID | GAGAATAGGT | SEQ ID | GTTGGTTGGT | SEQ ID | TGTGGTTGAA |
| NO: 470 | | NO: 556 | | NO: 642 | |
| SEQ ID | GAGAGGTATA | SEQ ID | GTTGGTTTAG | SEQ ID | TGTGGTTGAT |
| NO: 471 | | NO: 557 | | NO: 643 | |
| SEQ ID | GAGAGGTTAA | SEQ ID | GTTGTATGGT | SEQ ID | TGTGTAAGGT |
| NO: 472 | | NO: 558 | | NO: 644 | |
| SEQ ID | GAGAGGTTTT | SEQ ID | GTTGTGGGTA | SEQ ID | TGTGTGAGAA |
| NO: 473 | | NO: 559 | | NO: 645 | |
| SEQ ID | GAGGTTATGA | SEQ ID | GTTGTGTAGA | SEQ ID | TTAAGGTGGA |
| NO: 474 | | NO: 560 | | NO: 646 | |
| SEQ ID | GAGTTGGTTT | SEQ ID | GTTTAAGTGG | SEQ ID | TTAGTTAGGG |
| NO: 475 | | NO: 561 | | NO: 647 | |
| SEQ ID | GAGTTTGGAT | SEQ ID | GTTTAGAAGG | SEQ ID | TTATGGAGGG |
| NO: 476 | | NO: 562 | | NO: 648 | |
| SEQ ID | GATAAGGTAG | SEQ ID | GTTTATGTGG | SEQ ID | TTGAAATGGG |
| NO: 477 | | NO: 563 | | NO: 649 | |
| SEQ ID | GATAGGTGTG | SEQ ID | GTTTGAGGTA | SEQ ID | TTGGAAAAGG |
| NO: 478 | | NO: 564 | | NO: 650 | |
| SEQ ID | GATAGGTTGG | SEQ ID | GTTTGGTGGA | SEQ ID | TTGGATAGGT |
| NO: 479 | | NO: 565 | | NO: 651 | |
| SEQ ID | GATATGAGGA | SEQ ID | GTTTGTGAAG | SEQ ID | TTGGGTGAAA |
| NO: 480 | | NO: 566 | | NO: 652 | |
| SEQ ID | GATATGTGGT | SEQ ID | GTTTGTGGTT | SEQ ID | TTGGGTGGTT |
| NO: 481 | | NO: 567 | | NO: 653 | |
| SEQ ID | GATGGAAGGG | SEQ ID | GTTTTGTGTG | SEQ ID | TTGGGTGTGA |
| NO: 482 | | NO: 568 | | NO: 654 | |
| SEQ ID | GATGGAAGTT | SEQ ID | TAAAGAGGGT | SEQ ID | TTGGTTATGG |
| NO: 483 | | NO: 569 | | NO: 655 | |
| SEQ ID | GATTAAGGTG | SEQ ID | TAAAGGGTAG | SEQ ID | TTGGTTGGAT |
| NO: 484 | | NO: 570 | | NO: 656 | |
| SEQ ID | GATTGGGAAG | SEQ ID | TAAATGGAGG | SEQ ID | TTGGTTTGTG |
| NO: 485 | | NO: 571 | | NO: 657 | |
| SEQ ID | GATTGGGTGG | SEQ ID | TAAGGGAAGA | SEQ ID | TTGTGAGGAA |
| NO: 486 | | NO: 572 | | NO: 658 | |
| SEQ ID | GATTGGTGTA | SEQ ID | TAAGGGTGTA | SEQ ID | TTGTGGGTAG |
| NO: 487 | | NO: 573 | | NO: 659 | |
| SEQ ID | GATTGGTTTG | SEQ ID | TAAGTATGGG | SEQ ID | TTGTGGTATG |
| NO: 488 | | NO: 574 | | NO: 660 | |
| SEQ ID | GATTGTGGGT | SEQ ID | TAAGTGGGTA | SEQ ID | TTGTGGTTGT |
| NO: 489 | | NO: 575 | | NO: 661 | |
| SEQ ID | GATTTAAGGG | SEQ ID | TAGAAGTTGG | SEQ ID | TTGTGTGAGG |
| NO: 490 | | NO: 576 | | NO: 662 | |
| SEQ ID | GATTTGGGTT | SEQ ID | TAGATAGGTG | SEQ ID | TTTAGGGAAG |
| NO: 491 | | NO: 577 | | NO: 663 | |
| SEQ ID | GGAAAGTTGA | SEQ ID | TAGGGATGGG | SEQ ID | TTTGGATGGG |
| NO: 492 | | NO: 578 | | NO: 664 | |
| SEQ ID | GGAAATATGG | SEQ ID | TAGGGTAGAA | SEQ ID | TTTGGGATGG |
| NO: 493 | | NO: 579 | | NO: 665 | |
| SEQ ID | GGAAGGGAAG | SEQ ID | TAGGGTATAG | SEQ ID | TTTGGGTAAG |
| NO: 494 | | NO: 580 | | NO: 666 | |
| SEQ ID | GGAATGGAAT | SEQ ID | TAGGTGGGTT | SEQ ID | TTTGGTGTGT |
| NO: 495 | | NO: 581 | | NO: 667 | |
| SEQ ID | GGAATTTTGG | SEQ ID | TAGGTTGAAG | SEQ ID | TTTGGTTGAG |
| NO: 496 | | NO: 582 | | NO: 668 | |
| SEQ ID | GGAGGAATAT | SEQ ID | TAGGTTTGGG | SEQ ID | TTTGTAGGTG |
| NO: 497 | | NO: 583 | | NO: 669 | |
| SEQ ID | GGAGGATATG | SEQ ID | TAGTATGTGG | SEQ ID | TTTGTATGGG |
| NO: 498 | | NO: 584 | | NO: 670 | |
| SEQ ID | GGAGGTTAAT | SEQ ID | TAGTGTGGTT | SEQ ID | TTTGTGGGTT |
| NO: 499 | | NO: 585 | | NO: 671 | |
| SEQ ID | GGAGGTTAGG | SEQ ID | TAGTTGGGTG | SEQ ID | TTTTGAGGGT |
| NO: 500 | | NO: 586 | | NO: 672 | |
| SEQ ID | GGAGTTTGTT | SEQ ID | TAGTTGTAGG | | |
| NO: 501 | | NO: 587 | | | |
| SEQ ID | GGATAGGTGA | SEQ ID | TATAAGGTGG | | |
| NO: 502 | | NO: 588 | | | |

It will be appreciated by persons skilled in the art that numerous variations and/or modifications may be made to the above-described embodiments, without departing from the broad general scope of the present disclosure. The present embodiments are, therefore, to be considered in all respects as illustrative and not restrictive.

1. A method for creating an oligonucleotide sequence to represent digital data, the method comprising:
selecting from a first set of multiple oligonucleotide sequences one oligonucleotide sequence for each of multiple parts of the data, the multiple oligonucleotide sequences being configured to generate an electric time-domain signal from one oligonucleotide sequence that is distinguishable from the electric time-domain signal from another oligonucleotide sequence, the electric time-domain signal being indicative of an electric characteristic of one or more nucleotides present in an electric sensor at any one point in time; and
combining the one oligonucleotide sequence for each of multiple parts of the data into a single oligonucleotide sequence that represents a single oligonucleotide molecule to encode the digital data.

2. The method of claim 1, wherein the electric sensor comprises a nanopore.

3. The method of claim 1, wherein the method further comprises determining the first set by selecting the multiple oligonucleotide sequences from multiple candidate sequences based on a distance between a first candidate sequence and a second candidate sequence, wherein determining the first set comprises calculating the distance between a first simulated electric time-domain signal from the first candidate sequence and a second simulated electric time-domain signal from the second candidate sequence.

4. (canceled)

5. (canceled)

6. The method of claim 3, wherein calculating the distance comprises calculating an error of matching the first simulated electric time-domain signal to the second simulated electric time-domain signal subject to a time domain transformation that minimises the error.

7. (canceled)

8. (canceled)

9. The method of claim 1, wherein the method further comprises inserting a spacer sequence between each two of the multiple oligonucleotide sequences, wherein the spacer sequence is of sufficient length to generate, for a second oligonucleotide sequence from the first set, a predictable interference from the spacer sequence and not a preceding first oligonucleotide sequence.

10. (canceled)

11. The method of claim 9, wherein
the one or more nucleotides present in the electric sensor at any one point in time comprises a number f of nucleotides present in the electric sensor at any one point in time, and
the spacer sequence is of length kₛ with f≤kₛ≤2f.

12. (canceled)

13. The method of claim 9, wherein the method further comprises selecting the spacer sequence from a second set of spacer sequences comprising more than one spacer sequences to encode further digital data.

14. The method of claim 9, wherein the method further comprises repeating the method to create more than one oligonucleotide molecules comprising spacer sequences between oligonucleotide sequences, the spacer sequences being selected to create an index between the more than one oligonucleotide molecules.

15. The method of claim 9, wherein the method comprises repeating the method to create more than one oligonucleotide molecules comprising spacer sequences between oligonucleotide sequences, the spacer sequences being selected to obfuscate data encoded in the more than one oligonucleotide molecules.

16. The method of claim 1, wherein the method further comprises decoding the digital data from the single oligonucleotide molecule.

17. The method of claim 16, wherein decoding comprises:
capturing an electrical time-domain signal indicative of an electric characteristic of one or more nucleotides present in an electric sensor at any one point in time as the single oligonucleotide molecule passes through the sensor; and
identifying the multiple oligonucleotide sequences from the first set in the captured electrical time-domain signal, wherein identifying the multiple oligonucleotide sequences from the first set comprises matching the captured electrical time-domain signal against simulated electrical time-domain signals associated with the multiple oligonucleotide sequences in the first set.

18. (canceled)

19. The method of claim 16, wherein decoding further comprises:
identifying spacer sequences in the captured electrical time-domain signal;
splitting the captured electrical time-domain signal where the identified spacer sequences are identified;
identifying one of the multiple oligonucleotide sequences of the first set for each split.

20. (canceled)

21. The method of claim 1, wherein the method further comprises:
synthesising the molecule; and
adding the molecule to a product for verification of the product, wherein verification of the product comprises:
decoding the digital data from the molecule, and
performing a cryptographic operation in relation to the digital data and verify the product based on verification data.

22. (canceled)

23. A non-transitory computer-readable medium with program code stored thereon that, when executed by a computer, causes the computer to perform the method of claim 1.

24. A computer system for creating an oligonucleotide sequence to represent digital data, the computer system comprising:
data memory to store a first set of multiple oligonucleotide sequences; and
a processor configured to:
select from the first set of multiple oligonucleotide sequences one oligonucleotide sequence for each of multiple parts of the data, the multiple oligonucleotide sequences being configured to generate an electric time-domain signal from one oligonucleotide sequence that is distinguishable from the electric time-domain signal from another oligonucleotide sequence, the electric time-domain signal being indicative of an electric characteristic of one or more nucleotides present in an electric sensor at any one point in time; and
combine the one oligonucleotide sequence for each of multiple parts of the data into a single oligonucleotide sequence that represents a single oligonucleotide molecule to encode the digital data.

25. An oligonucleotide molecule that represents digital data, wherein the molecule comprises multiple oligonucleotide sequences combined into the molecule, wherein the multiple oligonucleotide sequences are configured to generate an electric time-domain signal from one oligonucleotide sequence that is distinguishable from the electric time-domain signal from another oligonucleotide sequence, the electric time-domain signal being indicative of an electric characteristic of one or more nucleotides present in an electric sensor at any one point in time.

26. The oligonucleotide molecule of claim 25, wherein the multiple oligonucleotide sequences combined into the molecule include two or more of the sequences provided in one of the following sets of nucleotide sequences:
a) SEQ ID NOs: 1 to 16;
b) SEQ ID NOs: 17 to 32;
c) SEQ ID NOs: 33 to 96;
d) SEQ ID NOs: 97 to 160;
e) SEQ ID NOs: 161 to 416; or
f) SEQ ID NOs: 417 to 676.

27. A kit for verifying a product's identity, comprising one or more oligonucleotide molecules of claim 25.

28. A method for manufacturing an identifiable product, the method comprising:
manufacturing the product;
selecting from a first set of multiple oligonucleotide sequences one oligonucleotide sequence for each of multiple parts of digital identification data, the multiple oligonucleotide sequences being configured to generate an electric time-domain signal from one oligonucleotide sequence that is distinguishable from the electric time-domain signal from another oligonucleotide sequence, the electric time-domain signal being indicative of an electric characteristic of one or more nucleotides present in an electric sensor at any one point in time; and
combining the one oligonucleotide sequence for each of multiple parts of the data into a single oligonucleotide sequence that represents a single oligonucleotide molecule to encode the digital identification data;
synthesising the oligonucleotide molecule; and
adding the synthesised oligonucleotide sequence to the product to allow decoding the digital identification data to verify the product's identity.

29. (canceled)

30. A method of verifying a product's identity, the method comprising:
providing a product to which a oligonucleotide molecule has been added,
obtaining an electrical signal indicative of a sequence of the oligonucleotide molecule;
selecting from a first set of multiple oligonucleotide sequences one oligonucleotide sequence for each of multiple parts of the electrical signal, the multiple oligonucleotide sequences being configured to generate an electric time-domain signal from one oligonucleotide sequence that is distinguishable from the electric time-domain signal from another oligonucleotide sequence, the electric time-domain signal being indicative of an electric characteristic of one or more nucleotides present in an electric sensor at any one point in time; and
decoding digital data encoded by the multiple oligonucleotide sequences to verify the product's identity based on the decoded digital data.

31. (canceled)

32. An identifiable product comprising:
one or more product constituents; and
a synthesised oligonucleotide molecule added to the one or more product constituents, wherein
the synthesised oligonucleotide molecule is represented by a single oligonucleotide sequence,
the single oligonucleotide sequence is a combination of oligonucleotide sequences comprising one oligonucleotide sequence selected for each of multiple parts of digital data from a first set of multiple oligonucleotide sequences to encode the digital data,
the multiple oligonucleotide sequences being configured to generate an electric time-domain signal from one oligonucleotide sequence that is distinguishable from the electric time-domain signal from another oligonucleotide sequence, the electric time-domain signal being indicative of an electric characteristic of one or more nucleotides present in an electric sensor at any one point in time; and
the digital data allows verification of the product's identity from decoding the digital data from the synthesised oligonucleotide molecule.

33. (canceled)

34. (canceled)

35. The method of claim 1, wherein the first set of multiple oligonucleotide sequences consists of:
a) SEQ ID NOs: 1 to 16;
b) SEQ ID NOs: 17 to 32;
c) SEQ ID NOs: 33 to 96;
d) SEQ ID NOs: 97 to 160;
e) SEQ ID NOs: 161 to 416; or
f) SEQ ID NOs: 417 to 672.
